SEQUENCE LISTING (69 sequences)

SEQ ID NO: 1 - protein, 33 aa, synthetic construct
  REGION 1..33: note = dual agonists
  PEPTIDE 1..33: note = peptide
HADGTFISDYSTILDNLAARDFINWLIQTKITD

SEQ ID NO: 2 - protein, 32 aa, synthetic construct
  REGION 1..32: note = dual agonists
  PEPTIDE 1..32: note = peptide
HADGTFISDYSTILDNLAARDFINWLIQTKIT

SEQ ID NO: 3 - protein, 42 aa, synthetic construct
  REGION 1..42: note = dual agonists
  PEPTIDE 1..42: note = peptide
HADGTFISDYSTILDNLAAKDFINWLIQTKITKNDWKHNITQ

SEQ ID NO: 4 - protein, 33 aa, synthetic construct
  REGION 1..33: note = dual agonists
  MOD_RES 2: note = MISC_FEATURE - X=Aib
  MOD_RES 13: note = MISC_FEATURE - X=Aib
HXDGTFISDYSTXLDNLAARDFINWLIQTKITK

SEQ ID NO: 5 - protein, 33 aa, synthetic construct
  REGION 1..33: note = dual agonists
  MOD_RES 2: note = MISC_FEATURE - X=Aib
  MOD_RES 16: note = MISC_FEATURE - X=Aib
HXDGTFISDYSTILDXLAARDFINWLIQTKITK

SEQ ID NO: 6 - protein, 33 aa, synthetic construct
  REGION 1..33: note = dual agonists
  MOD_RES 2: note = MISC_FEATURE - X=Aib
HXDGTFISDYSTILDNLAARDFIEWLIQTKITK

SEQ ID NO: 7 - protein, 32 aa, synthetic construct
  REGION 1..32: note = dual agonists
  MOD_RES 2: note = MISC_FEATURE - X=Aib
HXDGTFISDYSTILDNLAARDFIEWLIQTKIT

SEQ ID NO: 8 - protein, 33 aa, synthetic construct
  REGION 1..33: note = dual agonists
  MOD_RES 2: note = MISC_FEATURE - X=Aib
HXDGKFISDYSTILDNLAARDFINWLIQTKITK

SEQ ID NO: 9 - protein, 33 aa, synthetic construct
  REGION 1..33: note = dual agonists
  MOD_RES 2: note = MISC_FEATURE - X=Aib
HXDGTFKSDYSTILDNLAARDFINWLIQTKITK

SEQ ID NO: 10 - protein, 33 aa, synthetic construct
  REGION 1..33: note = dual agonists
  MOD_RES 2: note = MISC_FEATURE - X=Aib
HXDGTFISDYKTILDNLAARDFINWLIQTKITK

SEQ ID NO: 11 - protein, 33 aa, synthetic construct
  REGION 1..33: note = dual agonists
  MOD_RES 2: note = MISC_FEATURE - X=Aib
HXDGTFISDYSKILDNLAARDFINWLIQTKITK

SEQ ID NO: 12 - protein, 33 aa, synthetic construct
  REGION 1..33: note = dual agonists
  MOD_RES 2: note = MISC_FEATURE - X=Aib
HXDGTFISDYSTILKNLAARDFINWLIQTKITK

SEQ ID NO: 13 - protein, 42 aa, synthetic construct
  REGION 1..42: note = dual agonists
  MOD_RES 2: note = MISC_FEATURE - X=Aib
  MOD_RES 42: note = AMIDATION
HXDGTFISDYKTILDNLAARDFINWLIQTKGKKNDWKHNITQ

SEQ ID NO: 14 - protein, 42 aa, synthetic construct
  REGION 1..42: note = dual agonists
  PEPTIDE 1..42: note = peptide
HADGTFISDYSTILELLAARDFINWLIQTKITKNDWKHNITQ

SEQ ID NO: 15 - protein, 42 aa, synthetic construct
  REGION 1..42: note = dual agonists
  MOD_RES 16: note = MISC_FEATURE - X=Aib
HADGTFISDYSTILDXLAARDFINWLIQTKITKNDWKHNITQ

SEQ ID NO: 16 - protein, 33 aa, synthetic construct
  REGION 1..33: note = dual agonists
  MOD_RES 2: note = MISC_FEATURE - X=Aib
  MOD_RES 13: note = MISC_FEATURE - X=Aib
HXEGTFISDYSTXLDNLAARDFINWLIQTKITK

SEQ ID NO: 17 - protein, 33 aa, synthetic construct
  REGION 1..33: note = dual agonists
  MOD_RES 2: note = MISC_FEATURE - X=Aib
  MOD_RES 13: note = MISC_FEATURE - X=Aib
HXDGTFISDYSTXLDALAARDFIEWLIQTKITK

SEQ ID NO: 18 - protein, 33 aa, synthetic construct
  REGION 1..33: note = dual agonists
  MOD_RES 2: note = MISC_FEATURE - X=Aib
  MOD_RES 13: note = X = Aib
HXEGTFISDYSTXLDKLAARDFINWLIQTKITK

SEQ ID NO: 19 - protein, 33 aa, synthetic construct
  REGION 1..33: note = dual agonists
  MOD_RES 2: note = MISC_FEATURE - X=Aib
  MOD_RES 13: note = MISC_FEATURE - X=Aib
HXDGTFISDYSTXLDKLAARDFIEWLIQTKITK

SEQ ID NO: 20 - protein, 33 aa, synthetic construct
  REGION 1..33: note = dual agonists
  MOD_RES 2: note = MISC_FEATURE - X=Aib
  MOD_RES 13: note = MISC_FEATURE - X=Aib
HXDGTFISDYSTXLDKLAARDFINWLIETKITK

SEQ ID NO: 21 - protein, 33 aa, synthetic construct
  REGION 1..33: note = dual agonists
  MOD_RES 2: note = MISC_FEATURE - X=Aib
  MOD_RES 13: note = MISC_FEATURE - X=Aib
HXDGKFISDYSTXLDNLAARDFINWLIQTKITK

SEQ ID NO: 22 - protein, 33 aa, synthetic construct
  REGION 1..33: note = dual agonists
  MOD_RES 2: note = MISC_FEATURE - X=Aib
  MOD_RES 13: note = MISC_FEATURE - X=Aib
HXEGKFISDYSTXLDNLAARDFINWLIQTKITK

SEQ ID NO: 23 - protein, 33 aa, synthetic construct
  REGION 1..33: note = dual agonists
  MOD_RES 2: note = MISC_FEATURE - X=Aib
  MOD_RES 13: note = MISC_FEATURE - X=Aib
HXDGTFISDYKTXLDNLAARDFINWLIQTKITK

SEQ ID NO: 24 - protein, 33 aa, synthetic construct; identical to SEQ ID NO: 23
  REGION 1..33: note = dual agonists
  MOD_RES 1: note = ACETYLATION
  MOD_RES 2: note = MISC_FEATURE - X=Aib
  MOD_RES 13: note = MISC_FEATURE - X=Aib

SEQ ID NO: 25 - protein, 33 aa, synthetic construct
  REGION 1..33: note = dual agonists
  MOD_RES 2: note = MISC_FEATURE - X=Aib
  MOD_RES 13: note = MISC_FEATURE - X=Aib
HXDGTFISDYKTXLDNLAARDFINWLIQTKITD

SEQ ID NO: 26 - protein, 33 aa, synthetic construct
  REGION 1..33: note = dual agonists
  MOD_RES 2: note = MISC_FEATURE - X=Aib
HXDGTFISDYKTILDKLAARDFIEWLIATKITK

SEQ ID NO: 27 - protein, 33 aa, synthetic construct
  REGION 1..33: note = dual agonists
  MOD_RES 2: note = MISC_FEATURE - X=Aib
HXDGTFISDYKTILDNLAARDFINWLIETKITK

SEQ ID NO: 28 - protein, 33 aa, synthetic construct
  REGION 1..33: note = dual agonists
  MOD_RES 2: note = MISC_FEATURE - X=Aib
  MOD_RES 13: note = MISC_FEATURE - X=Aib
HXDGTFISDYKTXLDALAARDFIEWLIQTKITK

SEQ ID NO: 29 - protein, 33 aa, synthetic construct
  REGION 1..33: note = dual agonists
  MOD_RES 2: note = MISC_FEATURE - X=Aib
  MOD_RES 13: note = MISC_FEATURE - X=Aib
HXDGTFISDYSKXLDNLAARDFINWLIQTKITK

SEQ ID NO: 30 - protein, 33 aa, synthetic construct; identical to SEQ ID NO: 29
  REGION 1..33: note = dual agonists
  MOD_RES 1: note = ACETYLATION
  MOD_RES 2: note = MISC_FEATURE - X=Aib
  MOD_RES 13: note = MISC_FEATURE - X=Aib

SEQ ID NO: 31 - protein, 42 aa, synthetic construct
  REGION 1..42: note = dual agonists
  MOD_RES 2: note = MISC_FEATURE - X=Aib
  MOD_RES 13: note = MISC_FEATURE - X=Aib
HXDGTFISDYKTXLDNLAARDFINWLIQTKITKNDWKHNITQ

SEQ ID NO: 32 - protein, 42 aa, synthetic construct; identical to SEQ ID NO: 31
  REGION 1..42: note = dual agonists
  MOD_RES 1: note = ACETYLATION
  MOD_RES 2: note = MISC_FEATURE - X=Aib
  MOD_RES 13: note = MISC_FEATURE - X=Aib

SEQ ID NO: 33 - protein, 42 aa, synthetic construct
  REGION 1..42: note = dual agonists
  MOD_RES 13: note = MISC_FEATURE - X=Aib
HADGTFISDYSTXLDNLAARDFINWLIQTKITKNDWKHNITQ

SEQ ID NO: 34 - protein, 42 aa, synthetic construct
  REGION 1..42: note = dual agonists
  MOD_RES 2: note = MISC_FEATURE - X=Aib
  MOD_RES 13: note = MISC_FEATURE - X=Aib
HXDGTFISDYSTXLDNLAARDFINWLIQTKITKNDWKHNITQ

SEQ ID NO: 35 - protein, 42 aa, synthetic construct
  REGION 1..42: note = dual agonists
  MOD_RES 2: note = MISC_FEATURE - X=Aib
  MOD_RES 13: note = MISC_FEATURE - X=Aib
HXEGTFISDYSTXLDALAARDFINWLIQTKITKNDWKHNITQ

SEQ ID NO: 36 - protein, 42 aa, synthetic construct
  REGION 1..42: note = dual agonists
  MOD_RES 2: note = MISC_FEATURE - X=Aib
  MOD_RES 13: note = MISC_FEATURE - X=Aib
HXDGTFISDYSTXLDNLAAKDFINWLIQTKITKNDWKHNITQ

SEQ ID NO: 37 - protein, 42 aa, synthetic construct
  REGION 1..42: note = dual agonists
  MOD_RES 2: note = MISC_FEATURE - X=Aib
  MOD_RES 13: note = MISC_FEATURE - X=Aib
HXEGTFISDYSTXLDNLAAKDFINWLIQTKITKNDWKHNITQ

SEQ ID NO: 38 - protein, 33 aa, synthetic construct
  REGION 1..33: note = dual agonists
  PEPTIDE 1..33: note = peptide
HADGTFISDYSTILDNLAAKDFINWLIQTKITK

SEQ ID NO: 39 - protein, 33 aa, synthetic construct
  REGION 1..33: note = dual agonists
  REGION 1..33: note = MISC_FEATURE - peptide
HADGTFISDYSTILDNLAARDFINWLLAQKITK

SEQ ID NO: 40 - skipped

SEQ ID NO: 41 - protein, 42 aa, synthetic construct
  REGION 1..42: note = dual agonists
  PEPTIDE 1..42: note = peptide
HADGTFISDYSTILDELAARDFINWLIQTKITKNDWKHNITQ

SEQ ID NO: 42 - protein, 42 aa, synthetic construct
  REGION 1..42: note = dual agonists
  REGION 1..42: note = MISC_FEATURE - peptide
HADGTFISDYSTILDNLAARDFINWLIQTKITKAEWKHAITQ

SEQ ID NO: 43 - protein, 42 aa, synthetic construct
  REGION 1..42: note = dual agonists
  PEPTIDE 1..42: note = peptide
HADGTFISDYSTILDNLAARDFIEWLIQTKITKNDWKHNITQ

SEQ ID NO: 44 - protein, 42 aa, synthetic construct
  REGION 1..42: note = dual agonists
  PEPTIDE 1..42: note = peptide
HADGTFISDYSTILDNLAARDFINWLIETKITKNDWKHNITQ

SEQ ID NO: 45 - protein, 42 aa, synthetic construct
  REGION 1..42: note = dual agonists
  PEPTIDE 1..42: note = peptide
HADGTFISDYSTILDNLAARDFINWLIATKITKNDWKHNITQ

SEQ ID NO: 46 - protein, 42 aa, synthetic construct
  REGION 1..42: note = dual agonists
  MOD_RES 2: note = MISC_FEATURE - X=Aib
  MOD_RES 13: note = MISC_FEATURE - X=Aib
HXDGTFISDYKTXLDNLAARDFINWLIQTKITGNDWKHNITQ

SEQ ID NO: 47 - protein, 33 aa, synthetic construct
  REGION 1..33: note = dual agonists
  PEPTIDE 1..33: note = peptide
HADGTFISDYSTILDKLAAKDFINWLIQTKITK

SEQ ID NO: 48 - protein, 33 aa, synthetic construct
  REGION 1..33: note = dual agonists
  PEPTIDE 1..33: note = peptide
HADGTFISDYSTILDKLAARDFINWLIQTKITK

SEQ ID NO: 49 - protein, 33 aa, synthetic construct
  REGION 1..33: note = dual agonists
  PEPTIDE 1..33: note = peptide
HADGTFISDYSTILDNLAARDFINWLIQTKGKK

SEQ ID NO: 50 - protein, 34 aa, synthetic construct
  REGION 1..34: note = dual agonists
  MOD_RES 13: note = MISC_FEATURE - X = Aib
HADGTFISDYSTXLDNLAARDFINWLIQTKGKKK

SEQ ID NO: 51 - protein, 33 aa, synthetic construct
  REGION 1..33: note = dual agonists
  MOD_RES 13: note = MISC_FEATURE - X = Aib
HADGTFISDYSTXLDKLAARDFINWLIQTKGKK

SEQ ID NO: 52 - protein, 33 aa, synthetic construct
  REGION 1..33: note = dual agonists
  MOD_RES 2: note = MISC_FEATURE - X = Aib
HXDGTFISDYSTILDNLAARDFINWLIQTKITK

SEQ ID NO: 53 - protein, 33 aa, synthetic construct
  REGION 1..33: note = dual agonists
  PEPTIDE 1..33: note = peptide
HADGTFISDYSTILDKLAARDFINWLIQTKGKK

SEQ ID NO: 54 - protein, 33 aa, synthetic construct; identical to SEQ ID NO: 4
  REGION 1..33: note = dual agonists
  MOD_RES 2: note = MISC_FEATURE - X = Aib
  MOD_RES 13: note = MISC_FEATURE - X = Aib
  MOD_RES 33: note = AMIDATION

SEQ ID NO: 55 - protein, 33 aa, synthetic construct
  REGION 1..33: note = dual agonists
  PEPTIDE 1..33: note = peptide
HGEGSFGSDMSIALDKLAARDFVNWLLQTKITD

SEQ ID NO: 56 - protein, 42 aa, synthetic construct
  REGION 1..42: note = dual agonists
  REGION 1..42: note = MISC_FEATURE - hGIP
YAEGTFISDYSIAMDKIHQQDFVNWLLAQKGKKNDWKHNITQ

SEQ ID NO: 57 - protein, 33 aa, synthetic construct
  REGION 1..33: note = dual agonists
  REGION 1..33: note = MISC_FEATURE - hGLP-2
HADGSFSDEMNTILDNLAARDFINWLIQTKITD

SEQ ID NO: 58 - protein, 9 aa, synthetic construct
  REGION 1..9: note = dual agonists
  REGION 1..9: note = MISC_FEATURE - GIP(34-42)
NDWKHNITQ

SEQ ID NO: 59 - protein, 4 aa, synthetic construct
  REGION 1..4: note = dual agonists
  PEPTIDE 1..4: note = peptide
NDWK

SEQ ID NO: 60 - protein, 5 aa, synthetic construct
  REGION 1..5: note = dual agonists
  PEPTIDE 1..5: note = peptide
NDWKH

SEQ ID NO: 61 - protein, 6 aa, synthetic construct
  REGION 1..6: note = dual agonists
  PEPTIDE 1..6: note = peptide
NDWKHN

SEQ ID NO: 62 - protein, 7 aa, synthetic construct
  REGION 1..7: note = dual agonists
  PEPTIDE 1..7: note = peptide
NDWKHNI

SEQ ID NO: 63 - protein, 8 aa, synthetic construct
  REGION 1..8: note = dual agonists
  PEPTIDE 1..8: note = peptide
NDWKHNIT

SEQ ID NO: 64 - protein, 9 aa, synthetic construct
  REGION 1..9: note = dual agonists
  PEPTIDE 1..9: note = peptide
NDWKHNITQ

SEQ ID NO: 65 - protein, 9 aa, synthetic construct
  REGION 1..9: note = dual agonists
  PEPTIDE 1..9: note = peptide
AEWKHAITQ

SEQ ID NO: 66 - protein, 33 aa, synthetic construct
  REGION 1..33: note = dual agonists
  VAR_SEQ 2: note = MISC_FEATURE - X = A, K or Aib
  VAR_SEQ 3: note = MISC_FEATURE - X = D, K or E
  VAR_SEQ 5: note = MISC_FEATURE - X = T, K or S
  VAR_SEQ 7: note = MISC_FEATURE - X = K or I
  VAR_SEQ 8: note = MISC_FEATURE - X = K or S
  VAR_SEQ 9: note = MISC_FEATURE - X = D or K
  VAR_SEQ 10: note = MISC_FEATURE - X = Y or K
  VAR_SEQ 11: note = MISC_FEATURE - X = S, K or N
  VAR_SEQ 12: note = MISC_FEATURE - X = K or T
  VAR_SEQ 13: note = MISC_FEATURE - X = A, Aib, K or I
  VAR_SEQ 14: note = MISC_FEATURE - X = L or K
  VAR_SEQ 15: note = MISC_FEATURE - X = K, D or E
  VAR_SEQ 16: note = MISC_FEATURE - X = E, L, Aib, K, A or N
  VAR_SEQ 20: note = MISC_FEATURE - X = R or K
  VAR_SEQ 24: note = MISC_FEATURE - X = N or E
  VAR_SEQ 28: note = MISC_FEATURE - X = Q, E or A
  VAR_SEQ 31: note = MISC_FEATURE - X = I, G or omitted
  VAR_SEQ 32: note = MISC_FEATURE - X = T, K or omitted
  VAR_SEQ 33: note = MISC_FEATURE - X = K, G, D or omitted
HXXGXFXXXXXXXXXXLAAXDFIXWLIXTKXXX

SEQ ID NO: 67 - protein, 33 aa, synthetic construct
  REGION 1..33: note = dual agonists
  VAR_SEQ 2: note = MISC_FEATURE - X = K or Aib
  VAR_SEQ 3: note = MISC_FEATURE - X = D or E
  VAR_SEQ 5: note = MISC_FEATURE - X = T, K or S
  VAR_SEQ 8: note = MISC_FEATURE - X = K or S
  VAR_SEQ 9: note = MISC_FEATURE - X = D or K
  VAR_SEQ 10: note = MISC_FEATURE - X = Y or K
  VAR_SEQ 11: note = MISC_FEATURE - X = S or K
  VAR_SEQ 12: note = MISC_FEATURE - X = K or T
  VAR_SEQ 13: note = MISC_FEATURE - X = Aib or I
  VAR_SEQ 15: note = MISC_FEATURE - X = D or E
  VAR_SEQ 16: note = MISC_FEATURE - X = E, L, Aib, K, A or N
  VAR_SEQ 20: note = MISC_FEATURE - X = R or K
  VAR_SEQ 24: note = MISC_FEATURE - X = N or E
  VAR_SEQ 28: note = MISC_FEATURE - X = Q, E or A
  VAR_SEQ 31: note = MISC_FEATURE - X = I, G or omitted
  VAR_SEQ 32: note = MISC_FEATURE - X = T or omitted
  VAR_SEQ 33: note = MISC_FEATURE - X = K, G, D or omitted
HXXGXFIXXXXXXLXXLAAXDFIXWLIXTKXXX

SEQ ID NO: 68 - protein, 33 aa, synthetic construct; identical to SEQ ID NO: 66
  REGION 1..33: note = dual agonist
  MOD_RES 2: note = X = Aib
  VAR_SEQ 3: note = X = D, K or E
  VAR_SEQ 5: note = X=T, K or S
  VAR_SEQ 7: note = X = K or I
  VAR_SEQ 8: note = X = K or S
  VAR_SEQ 9: note = X = D or K
  VAR_SEQ 10: note = X = Y or K
  VAR_SEQ 11: note = X = S, K or N
  VAR_SEQ 12: note = X = K or T
  VAR_SEQ 13: note = X = A, Aib, K or I
  VAR_SEQ 14: note = X = L or K
  VAR_SEQ 15: note = X = K, D, or E
  VAR_SEQ 16: note = X = E, L, Aib, K, A or N
  VAR_SEQ 20: note = X = R or K
  VAR_SEQ 24: note = X = N or E
  VAR_SEQ 28: note = X = Q, E or A
  VAR_SEQ 31: note = X = I, G or omitted
  VAR_SEQ 32: note = X = T, K or omitted
  VAR_SEQ 33: note = X = K, G, D or omitted

SEQ ID NO: 69 - protein, 33 aa, synthetic construct; identical to SEQ ID NO: 67
  REGION 1..33: note = dual agonists
  MOD_RES 2: note = X = Aib
  VAR_SEQ 3: note = X = D or E
  VAR_SEQ 5: note = X = T, K or S
  VAR_SEQ 8: note = X = K or S
  VAR_SEQ 9: note = X = D or K
  VAR_SEQ 10: note = X = Y or K
  VAR_SEQ 11: note = X = S or K
  VAR_SEQ 12: note = X = K or T
  VAR_SEQ 13: note = X = Aib or I
  VAR_SEQ 15: note = X = D or E
  VAR_SEQ 16: note = X = E, L, Aib, K, A or N
  VAR_SEQ 20: note = X = R or K
  VAR_SEQ 24: note = X = N or E
  VAR_SEQ 28: note = X = Q, E or A
  VAR_SEQ 31: note = X = I, G or omitted
  VAR_SEQ 32: note = X = T or omitted
  VAR_SEQ 33: note = X = K, G, D or omitted

(19) United States
(12) Patent Application Publication
(10) Pub. No.: US 2025/0304643 A1
(43) Pub. Date: Oct. 2, 2025

(54) PEPTIDE DUAL AGONISTS OF GIPR AND GLP2R

(71) Applicant: BAINAN BIOTECH APS, København N (DK)

(72) Inventors: Ditte RIBER, Brønshøj (DK); Mette Marie ROSENKILDE, Hellerup (DK)

(21) Appl. No.: 18/718,706

(22) PCT Filed: Dec. 16, 2022
(86) PCT No.: PCT/EP2022/086362

§ 371 Date: Jun. 11, 2024

(30) Foreign Application Priority Data
Dec. 17, 2021 (EP) 21215608.7

(51) Int. Cl. C07K 14/605; A61K 38/00; A61K 47/54

(52) U.S. Cl. CPC C07K 14/605; A61K 47/542; A61K 38/00

(57) ABSTRACT

The present invention relates to peptide dual agonists; or co-agonists; of the GIPR (glucose-dependent insulinotropic polypeptide receptor) and the GLP-2R (glucagon-like peptide-2 receptor); and their use for treatment of bone disorders such as osteoporosis.

### TECHNICAL FIELD

The present invention relates to peptide dual agonists; or co-agonists; of the GIPR (glucose-dependent insulinotropic polypeptide receptor) and the GLP-2R (glucagon-like peptide-2 receptor); and their use for treatment of bone disorders such as osteoporosis.

### BACKGROUND

Gastrointestinal peptides and adipokines are critical signalling molecules involved in controlling whole-body energy homeostasis. These circulating hormones regulate a variety of biological responses such as hunger, satiety and glucose uptake. In vivo experiments have established that these hormones also regulate bone metabolism, while associations between these hormones and bone mass have been observed in human clinical studies.

Incretins are gastrointestinal hormones that help to regulate carbohydrate metabolism in response to food intake. The two main incretins are glucose-dependent insulinotropic polypeptide (GIP) and glucagon-like peptide-1 (GLP-1), both secreted by intestinal epithelial cells. Intestinal glucagon-like peptide-2 (GLP-2) is co-secreted along with GLP-1 upon nutrient ingestion.

Gastrointestinal hormones released after meal ingestion, such as GIP and GLP-2 have been shown to regulate bone turnover; GIP has a positive effect on bone, and GLP-2 regulates bone homeostasis and have a positive contribution to bone mass.

Osteoporosis can be defined as a combination of reduced bone mass and altered bone quality, resulting in decreased bone strength with an increased risk of fractures. Gastrointestinal hormones including GIP and GLP-2 have each been implicated in bone metabolism and as potential therapies for treating osteoporosis.

WO 2018/069442 and WO 2020/169792 both disclose dual peptide agonists of GIPR and GLP-2R, however, it is desired that peptide agonists with higher potency towards GIPR and GLP-2R are developed for clinical use.

### SUMMARY

Dual agonists that combine the properties of GIP and GLP-2 receptor (GIPR and GLP-2R) agonists are provided herewith. The present inventors have designed the herein provided peptide dual agonists that target selectively GIPR and GLP-2R, without targeting or by targeting to a lesser extent GLP-1R, thanks to the presence of a fatty acid at the indicated positions. The peptide dual agonists disclosed herein may also have higher potency compared to previously known dual peptide agonists of GIPR and GLP-2R, thanks to certain amino acid substitutions. Furthermore, the dual GIPR and GLP-2R agonists of the present invention, thanks to certain amino acid substitutions compared to native GIP and GLP-2 as well as previously known dual agonist peptides as well as the fatty acid positioning, have diminished capabilities to recruit β-arrestin to the GIPR and induces less GIPR internalization compared to human GIP. This may result in prolonged activation of the GIPR and thus a more sustained effect.

The role of GLP-1R in bone is uncertain. Instead, its role in obesity/diabetes is clear. Thus, better to avoid potential side effects/unwanted effects by removing GLP-1R agonism for use in bone-indications, especially for example in patients where glucose control is unnecessary or disadvantageous. However, it has been challenging to diminish or remove GLP-1R agonism without affecting potency towards GIPR and GLP-2R.

One aspect of the present disclosure relates to peptide dual agonist comprising or consisting of the sequence

(SEQ ID NO: 68)
HX₂X₃GX₅FX₇X₈X₉X₁₀X₁₁X₁₂X₁₃X₁₄X₁₅X₁₆LAAX₂₀DFIX₂₄W
LIX₂₈TKX₃₁X₃₂X₃₃-Z,

wherein
X₂ is Aib,
X₃ is D, K or E,
X₅ is T, K or S,
X₇ is K or I
X₈ is K or S
X₉ is D or K,
X₁₀ is Y or K,
X₁₁ is S, K or N,
X₁₂ is K or T
X₁₃ is A, Aib, K or I,
X₁₄ is L or K,
X₁₅ is K, D or E,
X₁₆ is E, L, Aib, K, A, N
X₂₀ is R or K,
X₂₄ is N or E,
X₂₈ is Q, E or A,
X₃₁ is I, G or omitted,
X₃₂ is T, K or omitted, and
X₃₃ is K, G, D or omitted,
wherein Z is a peptide comprising one or more amino acid residues of GIP (34-42)

(NDWKHNITQ; SEQ ID NO: 58),

wherein said peptide is modified by attaching at least one fatty acid molecule at the N-terminus and/or at one or more Lysine residues at any one of positions 1 to 15 of SEQ ID NO: 66, andwherein said peptide is an agonist of GIPR (glucose-dependent insulinotropic polypeptide receptor) and is an agonist of GLP-2R (glucagon-like peptide-2 receptor).

Another aspect of the present disclosure relates to a peptide dual agonist according to any of the preceding claims for use as a medicament.

A further aspect of the present disclosure relates to a peptide dual agonist according to any of the preceding for use in a method of inhibiting bone resorption and/or stimulating bone formation and/or for use in a method of treating a bone disorder and/or for use in a method of treating osteoporosis in an individual suffering from Duchenne muscular dystrophy (DMD) or cerebral Palsy.

### DESCRIPTION OF DRAWINGS

FIG. 1: β-arrestin 2 recruitment and receptor internalization of two selected dual GIPR/GLP-2R compounds of the present invention. (A) Human GIPR was transiently expressed in HEK293 cells and assessed for β-arrestin 2 recruitment and (B) the N-terminally SNAP-tagged human GIPR was transiently expressed in HEK293A cells and assessed for internalization following stimulation of two selected peptide dual agonists. Data are shown as mean±SEM. For the β-arrestin 2 recruitment data are n=3 independent experiments carried out in duplicate.

FIG. 2: (A) Time dependent changes in the bone turnover marker CTX (resorption) after SC injection of Compound no. 11 or vehicle presented as individual percentage of baseline sample taken at time 10 am, (B) Time dependent changes in bone turnover marker P1NP (formation) after SC injection of Compound no. 11 or vehicle presented as individual percentage of baseline sample taken at time 10 am, (C) Area under the curve (AUC) for CTX calculated using the individual percentage of baseline using y=70% as baseline (lowest point), (D) Area under the curve for P1NP calculated using the individual percentage of baseline using y=0% as baseline. P-value=0.4. All data are shown as mean±SEM, n=4

FIG. 3: Human GIPR expression following ligand incubation. Human GIPR was transiently expressed in COS-7 cells and preincubated with human GIP or two selected peptide dual agonists compounds. Afterwards the receptors present at the cell surface were labelled with ¹²⁵I-GIP. Data are shown as mean±SD from n=1 experiment carried out in duplicate.

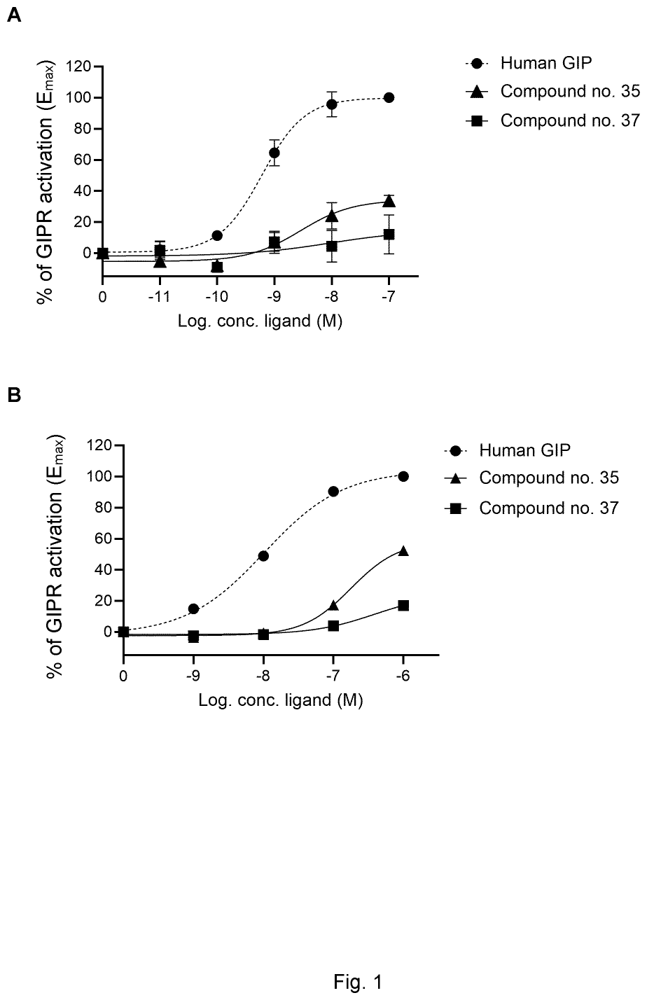

### DETAILED DESCRIPTION

### Definitions

The term “affinity” refers to the strength of binding between a ligand and its receptor.

The term “agonist” in the present context refers to a peptide as defined herein, capable of binding to and activating a receptor.

The term “dual agonist” or “co-agonist” refers to a peptide as defined herein, capable of binding to and activating at least two receptors, wherein the at least two receptors are different receptors. In the present context a dual agonist is an agonist of GIPR and an agonist of the GLP-2R. A dual agonist defined herewith may also have agonist activity towards additional receptors, whereby the dual agonist is an agonist of at least GIPR and GLP-2R.

An “amino acid residue” can be a natural or non-natural amino acid residue linked by peptide bonds or bonds different from peptide bonds. The amino acid residues can be in D-configuration or L-configuration. An amino acid residue comprises an amino terminal part (NH₂) and a carboxy terminal part (COOH) separated by a central part comprising a carbon atom, or a chain of carbon atoms, at least one of which comprises at least one side chain or functional group. NH₂ refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature as listed in J. Biol. Chem., 243:3552-59 (1969) and adopted in 37 C.F.R., section 1.822 (b) (2) belong to the group of amino acids listed herewith: Y,G,F,M,A,S,I,L,T,V,P,K,H,Q,E,W,R,D,N and C. Non-natural amino acids are those not listed immediately above. Also, non-natural amino acid residues include, but are not limited to, modified amino acid residues, L-amino acid residues, and stereoisomers of D-amino acid residues.

An “equivalent amino acid residue” refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, “equivalent amino acid residues” can be regarded as “conservative amino acid substitutions”.

Within the meaning of the term “equivalent amino acid substitution” as applied herein, one amino acid may be substituted for another, in one embodiment, within the groups of amino acids indicated herein below:

Amino acids having polar side chains (Asp, Glu, Lys, Arg, His, Asn, Gln, Ser, Thr, Pro, and Cys); Amino acids having non-polar side chains (Gly, Ala, Val, Leu, Ile, Phe, Trp, Tyr and Met); Amino acids having aliphatic side chains (Gly, Ala Val, Leu, Ile); Amino acids having cyclic side chains (Trp, His, Pro); Amino acids having aromatic side chains (Phe, Tyr, Trp); Amino acids having acidic, such as negatively charged side chains (Asp, Glu); Amino acids having basic, such as positively charged side chains (Lys, Arg, His); Amino acids having amide side chains (Asn, Gln); Amino acids having hydroxy side chains (Ser, Thr); Amino acids having sulphur-containing side chains (Cys, Met); Neutral, weakly hydrophobic amino acids (Pro, Ala, Gly, Ser, Thr); Hydrophilic, acidic amino acids (Gln, Asn, Glu, Asp); and Hydrophobic amino acids (Leu, Ile, Val).

Where the L or D form (optical isomers) has not been specified it is to be understood that the amino acid in question has the natural L form, cf. Pure & Appl. Chem. Vol. (56 (5) pp 595-624 (1984) or the D form, so that the peptides formed may be constituted of amino acids of L form, D form, or a sequence of mixed L forms and D forms.

A “functional variant” of a peptide is a peptide capable of performing essentially the same functions as the peptide it is a functional variant of. In particular, a functional variant can bind the same molecules, preferably with the same affinity, as the peptide it is a functional variant of.

A “bioactive agent” (i.e. a biologically active substance/agent) is any agent, drug, compound, composition of matter or mixture which provides some pharmacologic, often beneficial, effect that can be demonstrated in vivo or in vitro. It refers to the peptide sequences defined herewith, compounds or compositions comprising these and nucleic acid constructs encoding said peptides. As used herein, this term further includes any physiologically or pharmacologically active substance that produces a localized or systemic effect in an individual. A ‘bioactive agent’ as used herein denotes collectively a peptide, a nucleic acid construct encoding said peptide, and a composition comprising a peptide.

The terms “drug” and “medicament” as used herein include biologically, physiologically, or pharmacologically active substances that act locally or systemically in the human or animal body.

The terms “treatment” and “treating” as used herein refer to the management and care of a patient for the purpose of combating a condition, disease or disorder. The term is intended to include the full spectrum of treatments for a given condition from which the patient is suffering, and refer equally to curative therapy, prophylactic or preventative therapy and ameliorating or palliative therapy, such as administration of the peptide or composition for the purpose of: alleviating or relieving symptoms or complications; delaying the progression of the condition, partially arresting the clinical manifestations, disease or disorder; curing or eliminating the condition, disease or disorder; amelioration or palliation of the condition or symptoms, and remission (whether partial or total), whether detectable or undetectable; and/or preventing or reducing the risk of acquiring the condition, disease or disorder, wherein “preventing” or “prevention” is to be understood to refer to the management and care of a patient for the purpose of hindering the development of the condition, disease or disorder, and includes the administration of the active compounds to prevent or reduce the risk of the onset of symptoms or complications. The term “palliation”, and variations thereof, as used herein, means that the extent and/or undesirable manifestations of a physiological condition or symptom are lessened and/or time course of the progression is slowed or lengthened, as compared to not administering compositions of the present invention.

The term “Individual” refers to vertebrates, particular members of the mammalian species, preferably primates including humans. As used herein, ‘subject’ and ‘individual’ may be used interchangeably. Treatment of animals, such as mice, rats, dogs, cats, cows, horses, sheep and pigs, is, however, also within the scope of the present invention.

An “individual in need thereof” refers to an individual who may benefit from treatment. In one embodiment, said individual in need thereof is a diseased individual, wherein said disease may be a bone disorder.

A “treatment effect” or “therapeutic effect” is manifested if there is a change in the condition being treated, as measured by the criteria constituting the definition of the terms “treating” and “treatment.” There is a “change” in the condition being treated if there is at least 5% improvement, preferably 10% improvement, more preferably at least 25%, even more preferably at least 50%, such as at least 75%, and most preferably at least 100% improvement. The change can be based on improvements in the severity of the treated condition in an individual, or on a difference in the frequency of improved conditions in populations of individuals with and without treatment with the bioactive agent, or with the bioactive agent in combination with a pharmaceutical composition of the present invention.

A treatment according to the invention can be prophylactic, ameliorating and/or curative.

“Pharmacologically effective amount”, “pharmaceutically effective amount” or “physiologically effective amount” of a “bioactive agent” is the amount of a bioactive agent present in a pharmaceutical composition as described herein that is needed to provide a desired level of active agent in the bloodstream or at the site of action in an individual (e.g. the lungs, the gastric system, the colorectal system, prostate, etc.) to be treated to give an anticipated physiological response when such composition is administered.

“Co-administering” or “co-administration” as used herein refers to the administration of one or more agonists and a state-of-the-art pharmaceutical composition. The at least two components can be administered separately, sequentially or simultaneously.

“N-terminal region” as used herein, refers to the amino acid residues at positions 1 to 15 of a peptide dual agonist of the present disclosure.

GIP refers to glucose-dependent insulinotropic polypeptide, also known as Gastric Inhibitory Peptide (or polypeptide). As used herein the abbreviation hGIP is human GIP (Uniprot accession number P09681). GIP is derived from a 153-amino acid proprotein and circulates as a biologically active 42-amino acid peptide (positions 52-93). It is synthesized by K cells of the mucosa of the duodenum and the jejunum of the gastrointestinal tract.

Under physiological conditions the 42 amino acid hormone, GIP, is degraded by the enzyme dipeptidylpeptidase 4 (DPP-4), which cleaves at the third position of the GIP molecule to yield GIP3-42. GIP1-30 is produced as a result of post-translational processing. If GIP1-30 is secreted into the circulation in humans, the cleavage catalyzed by DPP-4 would result in GIP3-30.

The sequence of hGIP is:

(SEQ ID NO: 56)
YAEGTFISDYSIAMDKIHQQDFVNWLLAQKGKKNDWKHNITQ.

GIPR (or GIP receptor) refers to glucose-dependent insulinotropic polypeptide receptor(s). These seven-transmembrane proteins are found at least on beta-cells in the pancreas. As used herein the abbreviation hGIPR is human GIPR (Uniprot accession number P48546).

Several physiological effects of GIP have been identified. GIP is secreted from enteroendocrine K cells following nutrient intake and induces insulin secretion. The amount of insulin secreted is greater when glucose is administered orally than intravenously. GIP is also thought to have significant effects on fatty acid metabolism through stimulation of lipoprotein lipase activity in adipocytes. GIP recently appeared as a major player in bone remodelling, and deficiency in GIP receptors has been associated with a dramatic decrease in bone quality and a subsequent increase in fracture risk.

Glucagon-like peptide-2 (GLP-2) is a 33 amino acid peptide in humans created by specific post-translational proteolytic cleavage of proglucagon in a process that also liberates the related glucagon-like peptide-1 (GLP-1) and glucagon itself. GLP-2 is produced by the intestinal endocrine L cell and by various neurons in the central nervous system. Intestinal GLP-2 is co-secreted along with GLP-1 upon nutrient ingestion. When externally administered, GLP-2 produces a number of effects in humans and rodents, including intestinal growth, enhancement of intestinal function, reduction in bone breakdown and neuroprotection. GLP-2 and related analogs have potential as treatments for short bowel syndrome, Crohn's disease, osteoporosis and as adjuvant therapy during cancer chemotherapy.

The sequence of hGLP-2 is:

(SEQ ID NO: 57)
HADGSFSDEMNTILDNLAARDFINWLIQTKITD

The GLP-2 receptor (GLP-2R) is a G protein-coupled receptor superfamily member. GLP-2R is expressed in the gut and is closely related to the glucagon receptor (GCGR) and the receptor for GLP-1 (GLP-1R). As used herein the abbreviation hGLP-2R is human GLP-2R (e.g. Uniprot accession number 095838). As used herein the abbreviation hGLP-2 is human GLP-2 (Uniprot accession number not available). As used herein the abbreviation hGLP-1R is human GLP-1R (GLP-1 receptor) (e.g. Uniprot accession number P43220).

### GIPR and GLP-2R Peptide Dual Agonists

The present inventors have designed novel GIP and GLP-2 peptide analogues, which peptides are agonists of GIPR and of GLP-2R; i.e. are dual agonists of the GIPR and GLP-2R. A dual agonist of the GIPR and GLP-2R means that the peptide binds to and/or activates at least the GIPR and the GLP-2R. This makes them potentially useful in a range of therapeutic applications.

The peptides are designed by combining amino acids/amino acid stretches from the GIP peptide (SEQ ID NO: 56) and from the GLP-2 peptide (SEQ ID NO: 57). These amino acids are preferably involved in one or more of receptor binding, receptor affinity, receptor activity and/or otherwise relevant for the agonist activity of the peptides. In some embodiments, the peptides are designed to comprise amino acid residues which are not derived from any of GIP peptide (SEQ ID NO: 56) and GLP-2 peptide (SEQ ID NO: 57), but which may render said peptides of the present disclosure more stable and/or more potent and/or more selective compared to a peptide dual agonist comprising residues deriving solely from GIP peptide (SEQ ID NO: 56) and GLP-2 peptide (SEQ ID NO: 57).

It is an aspect to provide a peptide dual agonist comprising or consisting of the sequence

(SEQ ID NO: 66)
HX₂X₃GX₅FX₇X₈X₉X₁₀X₁₁X₁₂X₁₃X₁₄X₁₅X₁₆LAAX₂₀DFIX₂₄W
LIX₂₈TKX₃₁X₃₂X₃₃-Z,

wherein
X₂ is A, K or Aib,
X₃ is D, K or E,
X₅ is T, K or S,
X₇ is K or I
X₈ is K or S
X₉ is D or K,
X₁₀ is Y or K,
X₁₁ is S, K or N,
X₁₂ is K or T
X₁₃ is A, Aib, K or I,
X₁₄ is L or K,
X₁₅ is K, D or E,
X₁₆ is E, L, Aib, K, A, N
X₂₀ is R or K,
X₂₄ is N or E,
X₂₈ is Q, E or A,
X₃₁ is I, G or omitted,
X₃₂ is T, K or omitted, and
X₃₃ is K, G, D or omitted,
wherein Z is a peptide comprising one or more amino acid residues of GIP (34-42)

(NDWKHNITQ; SEQ ID NO: 58),

wherein said peptide is modified by attaching at least one fatty acid molecule at one or more amino acid residues at any one of positions 1 to 15 of SEQ ID NO: 66, and wherein said peptide is an agonist of GIPR (glucose-dependent insulinotropic polypeptide receptor) and is an agonist of GLP-2R (glucagon-like peptide-2 receptor).

It is an aspect to provide a peptide dual agonist comprising or consisting of the sequence

(SEQ ID NO: 68)
HX₂X₃GX₅FX₇X₈X₉X₁₀X₁₁X₁₂X₁₃X₁₄X₁₅X₁₆LAAX₂₀DFIX₂₄W
LIX₂₈TKX₃₁X₃₂X₃₃-Z,

wherein
X₂ is Aib,
X₃ is D, K or E,
X₅ is T, K or S,
X₇ is K or I
X₈ is K or S
X₉ is D or K,
X₁₀ is Y or K,
X₁₁ is S, K or N,
X₁₂ is K or T
X₁₃ is A, Aib, K or I,
X₁₄ is L or K,
X₁₅ is K, D or E,
X₁₆ is E, L, Aib, K, A, N
X₂₀ is R or K,
X₂₄ is N or E,
X₂₈ is Q, E or A,
X₃₁ is I, G or omitted,
X₃₂ is T, K or omitted, and
X₃₃ is K, G, D or omitted,
wherein Z is a peptide comprising one or more amino acid residues of GIP (34-42)

(NDWKHNITQ; SEQ ID NO: 58),

wherein said peptide is modified by attaching at least one fatty acid molecule at the N-terminus and/or at one or more Lysine residues at any one of positions 1 to 15 of SEQ ID NO: 68, and
wherein said peptide is an agonist of GIPR (glucose-dependent insulinotropic polypeptide receptor) and is an agonist of GLP-2R (glucagon-like peptide-2 receptor).

In one embodiment, the peptide dual agonist of the present disclosure has an amino acid sequence of

(SEQ ID NO: 69)
HX₂X₃GX₅FIX₈X₉X₁₀X₁₁X₁₂X₁₃X₁₄X₁₅X₁₆LAAX₂₀DFIX₂₄W
LIX₂₈TKX₃₁X₃₂X₃₃-Z,

wherein
X₂ is Aib,
X₃ is D or E,
X₅ is T, K or S,
X₈ is K or S
X₉ is D or K,
X₁₀ is Y or K,
X₁₁ is S or K,
X₁₂ is K or T
X₁₃ is Aib or I,
X₁₅ is D or E,
X₁₆ is E, L, Aib, K, A, N
X₂₀ is R or K,
X₂₄ is N or E,
X₂₈ is Q, E or A,
X₃₁ is I, G or omitted,
X₃₂ is T or omitted, and
X₃₃ is K, G, D or omitted.

The numbering in SEQ ID NO: 66, 67, 68 and 69 corresponds to the numbering of the amino acid residues in said sequence, such that:
H is the residue at position 1, X₂ is the residue at position 2, X₃ is the residue at position 3, G is the residue at position 4, X₅ is the residue at position 5, F is the residue at position 6, X₇ is the residue at position 7, X₈ is the residue at position 8, X₉ is the residue at position 9, X₁₀ is the residue at position 10, X₁₁ is the residue at position 11, X₁₂ is the residue at position 12, X₁₃ is the residue at position 13, X₁₄ is the residue at position 14, X₁₅ is the residue at position 15, X₁₆ is the residue at position 16, L is the residue at position 17, A is the residue at position 18, A is the residue at position 19, X₂₀ is the residue at position 20, D is the residue at position 21, F is the residue at position 22, I is the residue at position 23, X₂₄ is the residue at position 24, W is the residue at position 25, L is the residue at position 26, I is the residue at position 27, X₂₈ is the residue at position 28, T is the residue at position 29, K is the residue at position 30, X₃₁ is the residue at position 31, X₃₂ is the residue at position 32, X₃₃ is the residue at position 33 of said SEQ ID NO: 66 and 67.

Similarly, Z is a peptide that is numbered as the continuation of residues 1-33, thus the one or more amino acid residues of GIP (34-42) (NDWKHNITQ; SEQ ID NO:58) are for example numbered so that N is position 34, D is position 35, Wis position 36, K is position 37, H is position 38, N is position 39, I is position 40, T is position 41, Q is position 42.

However, in one embodiment Z may consist of only one amino acid residue, and in that case said amino acid residue is position 34, for example even if said amino acid residue is not an N. For example, in one embodiment Z may consist of only one K, and in that case said K is position 34.

In one embodiment, the present disclosure relates to a peptide dual agonist of GIPR and GLP-2R comprising the amino acid sequence SEQ ID NO: 66, 67, 68 or 69, or a functional variant thereof, or a functional fragment thereof.

SEQ ID NO: 66 may also be written as H(A/K/Aib)(D/K/E)G(T/K/S)F(K/I)(K/S)(D/K)(Y/K)(S/K/N)(K/T)(A/Aib/I/K)(L/K)(K/D/E)(E/L/Aib/K/A/N)LAA(R/K)DFI(N/E)WLI(Q/E/A)TK(I/G/omitted)(T/G/omitted)(K/G/D/omitted)-Z; or a functional variant thereof, or a functional fragment thereof.

Said peptide dual agonist being an agonist of GIPR and an agonist of GLP-2R implies that the peptide dual agonist is at an agonist of GIPR and of GLP-2R. It does not exclude that the peptide dual agonist can bind and/or activate further receptors. However, the peptide dual agonists of the present disclosure are designed to activate selectively GIPR and GLP-2R and in particular to activate GLP-1R to little or no extent.

The present inventors have in fact found that attachment of a fatty acid at the N-terminal region of a peptide dual agonist of the present disclosure, such as at anyone of the amino acid residues at positions 1 to 15 of a peptide of SEQ ID NO: 66, 67, 68 or 69, results in said peptide having little or no ability to bind and/or activate GLP-1R.

Reference to GIPR, GLP-2R, and GLP-1R as used herein throughout can be substituted with hGIPR, hGLP-2R, and hGLP-1R.

In one embodiment the peptide dual agonist comprises
a. amino acids from hGLP-2 (SEQ ID NO: 57) and/or hGIP (SEQ ID NO: 56) at the N-terminus,
b. a central/N-terminal stretch of amino acids primarily from hGIP (SEQ ID NO: 56),
c. a central/C-terminal stretch of amino acids primarily from hGLP-2 (SEQ ID NO: 57), and
d. optionally amino acids primarily from hGIP (SEQ ID NO: 56) at the C-terminus.

In one embodiment amino acids at positions 12 to 20 or 12 to 30 are primarily from hGLP-2 (SEQ ID NO: 57).

In one embodiment amino acids at positions 5 to 11 or 7 to 11 are primarily from hGIP (SEQ ID NO: 56).

In one embodiment when amino acids are defined as “primarily from” this means that at least 50% of the amino acids are from the peptide in question, such as at least 55%, such as at least 60%, such as at least 65%, such as at least 70%, such as at least 75%, such as at least 80%, such as at least 85%, such as at least 90%, such as at least 95%, such as 100% are from the peptide in question (i.e. from hGIP or from hGLP-2).

In one embodiment when amino acids are defined as “primarily from” this means that 50-55% of the amino acids are from the peptide in question, such as 55-60%, such as 60-65%, such as 65-70%, such as 70-75%, such as 75-80%, such as 80-85%, such as 85-90%, such as 90-95%, such as 95-100% are from the peptide in question (i.e. from hGIP or from hGLP-2).

When an amino acid is from hGIP or from hGLP-2, this means that the amino acid at a certain position of the peptide dual agonist corresponds to the amino acid at the same position of hGIP or hGLP-2. For instance, if the amino acid at position 1 is from GIP this means that the amino acid is “Y”. If the amino acid at position 1 is from GLP-2 this means that the amino acid is “H”.

In one embodiment the peptide dual agonist of the present disclosure comprises or consists of 20 to 43 consecutive amino acids; such as 20 to 22 amino acids, such as 22 to 24 amino acids, such as 24 to 26 amino acids, such as 26 to 28 amino acids, such as 28 to 30 amino acids, such as 30 to 32 amino acids, such as 32 to 34 amino acids, such as 34 to 36 amino acids, such as 36 to 38 amino acids, such as 38 to 40 amino acids, such as 40 to 42, such as 38 to 43 consecutive amino acids as defined herein.

In one embodiment the peptide dual agonist of the present disclosure comprises or consists of 20 to 43 consecutive amino acids; such as 20, 21, 22, 23, 24, 25, 26, 27, 28, 29, 30, 31, 32, 33, 34, 35, 36, 37, 38, 39, 40, 41, 42 or 43 consecutive amino acids.

In one embodiment the peptide dual agonist of the present disclosure comprises or consists of 20 to 33 consecutive amino acids, such as 20-21, such as 21-22, such as 22-23, such as 23-24, such as 24-25, such as 25-26, such as 26-27, such as 27-28, such as 28-29, such as 29-30, such as 30-31, such as 31-32, such as 32-33, such as 33-34, such as 34-35, such as 35-36, such as 36-37, such as 37-38, such as 38-39, such as 39-40, such as 40-41, such as 41-42, such as 42-43 consecutive amino acids.

In one embodiment the peptide dual agonist of the present disclosure comprises or consists of 30 consecutive amino acids.

In one embodiment the peptide dual agonist of the present disclosure comprises or consists of 32 consecutive amino acids.

In one embodiment the peptide dual agonist of the present disclosure comprises or consists of 33 consecutive amino acids.

Preferably, in one embodiment the peptide dual agonist of the present disclosure comprises or consists of at least 33 consecutive amino acids, but even a shorter peptide sequence may be used.

In one embodiment the peptide dual agonist of the present disclosure comprises or consists of 42 consecutive amino acids.

In one embodiment the peptide dual agonist of the present disclosure comprises or consists of 43 consecutive amino acids.

In one embodiment the peptide dual agonist of the present disclosure is modified by attaching at least one fatty acid molecule at any one of positions 2 to 15 of SEQ ID NO: 66, 67, 68 or 69.

In one embodiment the peptide dual agonist of the present disclosure comprises or consists of the amino acid sequence SEQ ID NO: 66, wherein
X₂ is K or Aib,
X₃ is D or E,
X₅ is Tor K,
X₇ is I,
X₁₁ is S or K,
X₁₃ is Aib or I,
X₁₅ is D or E,
X₁₆ E, L, Aib, K, A, N
X₂₄ is N or E,
X₂₈ is Q, E or A,
X₃₁ is I, G or omitted,
X₃₂ is T or omitted.

It is also an aspect to provide a peptide dual agonist comprising or consisting of the sequence

(SEQ ID NO: 67)
HX₂X₃GX₅FIX₈X₉X₁₀X₁₁X₁₂X₁₃LX₁₅X₁₆LAAX₂₀DFIX₂₄WLI
X₂₈TKX₃₁X₃₂X₃₃-Z,

wherein
X₂ is K or Aib,
X₃ is Dor E,
X₅ is T, K or S,
X₈ is K or S
X₉ is D or K,
X₁₀ is Y or K,
X₁₁ is S or K,
X₁₂ is K or T
X₁₃ is Aib or I,
X₁₅ is D or E,
X₁₆ is E, L, Aib, K, A, N
X₂₀ is R or K,
X₂₄ is N or E,
X₂₈ is Q, E or A,
X₃₁ is I, G or omitted,
X₃₂ is T or omitted, and
X₃₃ is K, G, D or omitted,
wherein Z is a peptide comprising one or more amino acid residues of GIP (34-42)

(NDWKHNITQ; SEQ ID NO: 58),

wherein said peptide is modified by attaching at least one fatty acid molecule at one or more amino acid residues at any one of positions 1 to 15 of SEQ ID NO: 67, and
wherein said peptide is an agonist of GIPR (glucose-dependent insulinotropic polypeptide receptor) and is an agonist of GLP-2R (glucagon-like peptide-2 receptor).

Thus, in one embodiment, the peptide dual agonist of the present disclosure comprises or consists of the amino acid sequence SEQ ID NO: 67, which may also be written as H(K/Aib)(D/E)G(T/K/S)FI(K/S)(D/K)(Y/K)(S/K)(K/T)(Aib/I)L(D/E)(E/L/Aib/K/A/N)LAA(R/K) DFI(N/E)WLI(Q/E/A)TK(I/G/omitted)(T/omitted)(K/G/D/omitted)-Z; or a functional variant thereof, or a functional fragment thereof.

In one embodiment the peptide dual agonist of the present disclosure comprises or consists of the amino acid sequence SEQ ID NO: 66, 67, 68 or 69, wherein
X₃ is D,
X₇ is I,
X₁₅ is D,
X₃₁ is I, and
X₃₂ is T.

In one embodiment the peptide dual agonist of the present disclosure comprises or consists of the amino acid sequence SEQ ID NO: 66, 67, 68 or 69, wherein X₂ is 2-Aminoisobutyric acid (Aib). Thus, in one embodiment, Alanine at position 2 of hGIP, hGLP-2 or hGLP-1 is substituted with 2-Aminoisobutyric acid. Presence of Aib at position 2 protects the dual agonist peptide from DPP-IV cleavage. In addition, presence of Aib at position 2 may also improve potency.

In one embodiment the peptide dual agonist of the present disclosure comprises or consists of the amino acid sequence SEQ ID NO: 66, 67, 68 or 69, wherein X₁₃ is Aib. Thus, in one embodiment, Alanine at position 13 of hGIP, or Isoleucine at position 13 of hGLP-2 are substituted with 2-Aminoisobutyric acid. Presence of Aib at position 13 increases potency on at least one of GIPR and GLP-2R.

In general, presence of one or more Aib residues makes the 3D structure of the peptide dual agonists of the present disclosure more rigid, which may improve potency and selectivity on GIPR and/or GLP-2R.

In one embodiment the peptide dual agonist of the present disclosure comprises or consists of the amino acid sequence SEQ ID NO: 66, 67, 68 or 69, wherein X₁₆ is A, Aib, L, E. Thus, in one embodiment, Asparagine (N) at position 16 of GLP-2 is substituted with any of A, Aib, L or E. It is found that substituting N with any of A, Aib, L or E improves physical stability of the peptide dual agonists of the present disclosure.

In one embodiment the peptide dual agonist of the present disclosure comprises or consists of the amino acid sequence SEQ ID NO: 66, 67, 68 or 69, wherein X₂₀ is K.

In one embodiment the peptide dual agonist of the present disclosure comprises or consists of the amino acid sequence SEQ ID NO: 66, 67, 68 or 69, wherein one or more Asparagine (N) and/or Glutamine (Q) residues of hGIP and/or hGLP-2 are substituted, such as wherein any one of residues N16 of hGLP-2, N24 of hGIP and hGLP-2 and Q28 of GLP-2 are substituted. These residues may for example be substituted with A, Aib, L, E or K, such as with E, A or K, such as with E or A.

Thus, in one embodiment the peptide dual agonist of the present disclosure comprises or consists of the amino acid sequence SEQ ID NO: 66, 67, 68 or 69, wherein X₂ is Aib and X₁₃ is Aib.

In one embodiment the peptide dual agonist of the present disclosure comprises or consists of the amino acid sequence SEQ ID NO: 66, 67, 68 or 69, wherein X₂ is Aib and X₁₁ is K.

In one embodiment the peptide dual agonist of the present disclosure comprises or consists of the amino acid sequence SEQ ID NO: 66, 67, 68 or 69, wherein X₂ is Aib, X₅ is K and X₁₃ is Aib.

In one embodiment the peptide dual agonist of the present disclosure comprises or consists of the amino acid sequence SEQ ID NO: 66, 67, 68 or 69, wherein X₂ is Aib, X₁₁ is K and X₁₃ is Aib.

In one embodiment the peptide dual agonist of the present disclosure comprises or consists of the amino acid sequence SEQ ID NO: 66, 67, 68 or 69, wherein X₂ is Aib, X₁₁ is K and X₂₄ is E.

In one embodiment X₃₁X₃₂X₃₃ is selected from the group consisting of ITD, GKK, GTD, IKD and ITK. In one embodiment X₃₁X₃₂X₃₃ is ITD. In one embodiment X₃₁X₃₂X₃₃ is GKK.

In one embodiment the peptide dual agonist of the present disclosure comprises or consists of the amino acid sequence SEQ ID NO: 66, 67, 68 or 69, wherein Z is selected from the group consisting of: ND, NDW, KDW, NDW, NDK, NDWK, NDWKH, NDWKHN, NDWKHNI, NDWKHNIT, NDWKHNITQ, and AEWKHAITQ or a variant comprising one amino acid substitution.

In one embodiment, the peptide dual agonist of the present disclosure comprises or consists of the amino acid sequence SEQ ID NO: 66, 67, 68 or 69, wherein Z is a short amino acid sequence, such as wherein Z is K.

In one embodiment, the peptide dual agonist of the present disclosure comprises or consists of the amino acid sequence SEQ ID NO: 66, 67, 68 or 69, wherein Z is a long amino acid sequence, such as wherein Z is selected from the group consisting of.

(SEQ ID NO: 65)
AEWKHAITQ
and
(SEQ ID NO: 64)
NDWKHNITQ.

In one embodiment the peptide dual agonist of the present disclosure is selected from the group consisting of

Compound no. 9
SEQ ID NO: 8
H-Aib-DGKFISDYSTILDNLAARDFINWLIQTKITK,
Compound no. 10
SEQ ID NO: 9
H-Aib-DGTFKSDYSTILDNLAARDFINWLIQTKITK,
Compound no. 11
SEQ ID NO: 10
H-Aib-DGTFISDYKTILDNLAARDFINWLIQTKITK,
Compound no. 12
SEQ ID NO: 11
H-Aib-DGTFISDYSKILDNLAARDFINWLIQTKITK,
Compound no. 13
SEQ ID NO: 12
H-Aib-DGTFISDYSTILKNLAARDFINWLIQTKITK,
Compound no. 14
SEQ ID NO: 13
H-Aib-DGTFISDYKTILDNLAARDFINWLIQTKGKKNDWKHNITQ,
Compound no. 24
SEQ ID NO: 21
H-Aib-DGKFISDYST-Aib-LDNLAARDFINWLIQTKITK,
Compound no. 25
SEQ ID NO: 21
H-Aib-DGKFISDYST-Aib-LDNLAARDFINWLIQTKITK,
Compound no. 26
SEQ ID NO: 8
H-Aib-DGKFISDYSTILDNLAARDFINWLIQTKITK,
Compound no. 27
SEQ ID NO: 22
H-Aib-EGKFISDYST-Aib-LDNLAARDFINWLIQTKITK,
Compound no. 28
SEQ ID NO: 21
H-Aib-DGKFISDYST-Aib-LDNLAARDFINWLIQTKITK,
Compound no. 29
SEQ ID NO: 23
H-Aib-DGTFISDYKT-Aib-LDNLAARDFINWLIQTKITK,
Compound no. 30
SEQ ID NO: 25
H-Aib-DGTFISDYKT-Aib-LDNLAARDFINWLIQTKITD,
Compound no. 31
SEQ ID NO: 26
H-Aib-DGTFISDYKTILDKLAARDFIEWLIATKITK,
Compound no. 32
SEQ ID NO: 27
H-Aib-DGTFISDYKTILDNLAARDFINWLIETKITK,
Compound no. 33
SEQ ID NO: 28
H-Aib-DGTFISDYKT-Aib-LDALAARDFIEWLIQTKITK,
Compound no. 34
SEQ ID NO: 24
Ac-H-Aib-DGTFISDYKT-Aib-LDNLAARDFINWLIQTKITK,
Compound no. 35
SEQ ID NO: 29
H-Aib-DGTFISDYSKAib-LDNLAARDFINWLIQTKITK,
Compound no. 36
SEQ ID NO: 30
Ac-H-Aib-DGTFISDYSKAib-LDNLAARDFINWLIQTKITK,
Compound no. 37
SEQ ID NO: 31
H-Aib-DGTFISDYKTAib-LDNLAARDFINWLIQTKITKNDWKHNITQ,
Compound no. 38
SEQ ID NO: 32
Ac-H-Aib-DGTFISDYKT-Aib-LDNLAARDFINWLIQTKITKNDWKHN
ITQ,
Compound no. 54
SEQ ID NO: 46
H-Aib-DGTFISDYKT-Aib-LDNLAARDFINWLIQTKITGNDWKHN
ITQ,
Compound no. 1
SEQ ID NO: 1
HADGTFISDYSTILDNLAARDFINWLIQTKITD,
Compound no. 2
SEQ ID NO: 2
HADGTFISDYSTILDNLAARDFINWLIQTKIT,
Compound no. 45
SEQ ID NO: 38
HADGTFISDYSTILDNLAAKDFINWLIQTKITK,
Compound no. 3
SEQ ID NO: 3
HADGTFISDYSTILDNLAAKDFINWLIQTKITKNDWKHNITQ,
Compound no. 46
SEQ ID NO: 39
HADGTFISDYSTILDNLAARDFINWLLAQKITK,
Compound no. 4
SEQ ID NO: 1
HADGTFISDYSTILDNLAARDFINWLIQTKITD,
Compound no. 5
SEQ ID NO: 4
H-Aib-DGTFISDYST-Aib-LDNLAARDFINWLIQTKITK,
Compound no. 6
SEQ ID NO: 5
H-Aib-DGTFISDYSTILD-Aib-LAARDFINWLIQTKITK,
Compound no. 7
SEQ ID NO: 6
H-Aib-DGTFISDYSTILDNLAARDFIEWLIQTKITK,
Compound no. 8
SEQ ID NO: 7
H-Aib-DGTFISDYSTILDNLAARDFIEWLIQTKIT,
Compound no. 15
SEQ ID NO: 14
HADGTFISDYSTILELLAARDFINWLIQTKITKNDWKHNITQ,
Compound no. 48
SEQ ID NO: 41
HADGTFISDYSTILDELAARDFINWLIQTKITKNDWKHNITQ,
Compound no. 49
SEQ ID NO: 42
HADGTFISDYSTILDNLAARDFINWLIQTKITKAEWKHAITQ,
Compound no. 50
SEQ ID NO: 43
HADGTFISDYSTILDNLAARDFIEWLIQTKITKNDWKHNITQ,
Compound no. 51
SEQ ID NO: 44
HADGTFISDYSTILDNLAARDFINWLIETKITKNDWKHNITQ,
Compound no. 52
SEQ ID NO: 45
HADGTFISDYSTILDNLAARDFINWLIATKITKNDWKHNITQ,
Compound no. 53
SEQ ID NO: 33
HADGTFISDYSTAibLDNLAARDFINWLIQTKITKNDWKHNITQ,
Compound no. 16
SEQ ID NO: 15
HADGTFISDYSTILDAibLAARDFINWLIQTKITKNDWKHNITQ,
Compound no. 17
SEQ ID NO: 4
H-Aib-DGTFISDYST-Aib-LDNLAARDFINWLIQTKITK,
Compound no. 19
SEQ ID NO: 16
H-Aib-EGTFISDYST-Aib-LDNLAARDFINWLIQTKITK,
Compound no. 20
SEQ ID NO: 17
H-Aib-DGTFISDYST-Aib-LDALAARDFIEWLIQTKITK,
Compound no. 21
SEQ ID NO: 18
H-Aib-EGTFISDYST-Aib-LDKLAARDFINWLIQTKITK,
Compound no. 22
SEQ ID NO: 19
H-Aib-DGTFISDYST-Aib-LDKLAARDFIEWLIQTKITK,
Compound no. 23
SEQ ID NO: 20
H-Aib-DGTFISDYST-Aib-LDKLAARDFINWLIETKITK,
Compound no. 39
SEQ ID NO: 33
HADGTFISDYSTAibLDNLAARDFINWLIQTKITKNDWKHNITQ,
Compound no. 40
SEQ ID NO: 34
H-Aib-DGTFISDYST-Aib-
LDNLAARDFINWLIQTKITKNDWKHNITQ,
Compound no. 41
SEQ ID NO: 35
H-Aib-EGTFISDYST-Aib-
LDALAARDFINWLIQTKITKNDWKHNITQ,
Compound no. 42
SEQ ID NO: 36
H-Aib-DGTFISDYST-Aib-
LDNLAAKDFINWLIQTKITKNDWKHNITQ,
Compound no. 43
SEQ ID NO: 37
H-Aib-EGTFISDYST-Aib-
LDNLAAKDFINWLIQTKITKNDWKHNITQ,
and
Compound no. 44
SEQ ID NO: 4
H-Aib-DGTFISDYST-Aib-LDNLAARDFINWLIQTKITK.

or a functional variant thereof, or a functional fragment thereof, for example wherein said functional variant thereof comprises 1, 2 or 3 individual amino acid substitutions compared to any one of the above amino acid sequences.

In one embodiment the peptide dual agonist of the present disclosure is selected from the group consisting of

SEQ ID NO: 4
H-Aib-DGTFISDYST-Aib-LDNLAARDFINWLIQTKITK,
SEQ ID NO: 5
H-Aib-DGTFISDYSTILD-Aib-LAARDFINWLIQTKITK,
SEQ ID NO: 8
H-Aib-DGKFISDYSTILDNLAARDFINWLIQTKITK,
SEQ ID NO: 10
H-Aib-DGTFISDYKTILDNLAARDFINWLIQTKITK,
SEQ ID NO: 11
H-Aib-DGTFISDYSKILDNLAARDFINWLIQTKITK,
SEQ ID NO: 13
H-Aib-DGTFISDY-KTILDNLAARDFINWLIQTKGKKNDWKHNITQ,
SEQ ID NO: 4
H-Aib-DGTFISDYST-Aib-LDNLAARDFINWLIQTKITK,
SEQ ID NO: 16
H-Aib-EGTFISDYST-Aib-LDNLAARDFINWLIQTKITK,
SEQ ID NO: 17
H-Aib-DGTFISDYST-Aib-LDALAARDFIEWLIQTKITK,
SEQ ID NO: 19
H-Aib-DGTFISDYST-Aib-LDKLAARDFIEWLIQTKITK,
SEQ ID NO: 21
H-Aib-DGKFISDYST-Aib-LDNLAARDFINWLIQTKITK,
SEQ ID NO: 23
H-Aib-DGTFISDYKT-Aib-LDNLAARDFINWLIQTKITK,
SEQ ID NO: 28
H-Aib-DGTFISDYKT-Aib-LDALAARDFIEWLIQTKITK,
SEQ ID NO: 31
H-Aib-DGTFISDY-KTAib-
LDNLAARDFINWLIQTKITKNDWKHNITQ,
SEQ ID NO: 34
H-Aib-DGTFISDYST-Aib-
LDNLAARDFINWLIQTKITKNDWKHNITQ,
SEQ ID NO: 35
H-Aib-EGTFISDYST-Aib-
LDALAARDFINWLIQTKITKNDWKHNITQ,
SEQ ID NO: 36
H-Aib-DGTFISDYST-Aib-
LDNLAAKDFINWLIQTKITKNDWKHNITQ,
SEQ ID NO: 44
H-Aib-DGTFISDYST-Aib-LDNLAARDFINWLIQTKITK,
and
SEQ ID NO: 26
H-Aib-DGTFISDYKTILDKLAARDFIEWLIATKITK,

or a functional variant thereof, or a functional fragment thereof; for example wherein said functional variant thereof comprises 1, 2 or 3 individual amino acid substitutions compared to any one of the above amino acid sequences.

In one aspect, the present disclosure relates to a peptide dual agonist comprising or consisting of an amino acid sequence selected from the group consisting of:

SEQ ID NO: 8
H-Aib-DG-KFISDYSTILDNLAARDFINWLIQTKITK,
SEQ ID NO: 9
H-Aib-DGTF-KSDYSTILDNLAARDFINWLIQTKITK,
SEQ ID NO: 10
H-Aib-DGTFISDY-KTILDNLAARDFINWLIQTKITK,
SEQ ID NO: 11
H-Aib-DGTFISDYSKILDNLAARDFINWLIQTKITK,
SEQ ID NO: 12
H-Aib-DGTFISDYSTILKNLAARDFINWLIQTKITK,
SEQ ID NO: 13
H-Aib-DGTFISDYKTILDNLAARDFINWLIQTKGKKNDWKHNITQ,
SEQ ID NO: 21
H-Aib-DG-KFISDYST-Aib-LDNLAARDFINWLIQTKITK,
SEQ ID NO: 22
H-Aib-EG-KFISDYST-Aib-LDNLAARDFINWLIQTKITK,
SEQ ID NO: 23
H-Aib-DGTFISDY-KT-Aib-LDNLAARDFINWLIQTKITK,
SEQ ID NO: 25
H-Aib-DGTFISDY-KT-Aib-LDNLAARDFINWLIQTKITD,
SEQ ID NO: 26
H-Aib-DGTFISDY-KTILDKLAARDFIEWLIATKITK,
SEQ ID NO: 27
H-Aib-DGTFISDY-KTILDNLAARDFINWLIETKITK,
SEQ ID NO: 28
H-Aib-DGTFISDY-KT-Aib-LDALAARDFIEWLIQTKITK,
SEQ ID NO: 24
Ac-H-Aib-DGTFISDY-KT-Aib-LDNLAARDFINWLIQTKITK,
SEQ ID NO: 29
H-Aib-DGTFISDYS-KAib-LDNLAARDFINWLIQTKITK,
SEQ ID NO: 30
Ac-H-Aib-DGTFISDYS-KAib-LDNLAARDFINWLIQTKITK,
SEQ ID NO: 31
H-Aib-DGTFISDY-KTAib-
LDNLAARDFINWLIQTKITKNDWKHNITQ,
SEQ ID NO: 32
Ac-H-Aib-DGTFISDY-KT-Aib-
LDNLAARDFINWLIQTKITKNDWKHNITQ,
SEQ ID NO: 46
H-Aib-DGTFISDY-KT-Aib-
LDNLAARDFINWLIQTKITGNDWKHNITQ,
SEQ ID NO: 5
H-Aib-DGTFISDYSTILD-Aib-LAARDFINWLIQTKITK,
SEQ ID NO: 6
H-Aib-DGTFISDYSTILDNLAARDFIEWLIQTKITK,
SEQ ID NO: 7
H-Aib-DGTFISDYSTILDNLAARDFIEWLIQTKIT,
SEQ ID NO: 44
H-Aib-DGTFISDYST-Aib-LDNLAARDFINWLIQTKITK,
SEQ ID NO: 16
H-Aib-EGTFISDYST-Aib-LDNLAARDFINWLIQTKITK,
SEQ ID NO: 17
H-Aib-DGTFISDYST-Aib-LDALAARDFIEWLIQTKITK,
SEQ ID NO: 18
H-Aib-EGTFISDYST-Aib-LDKLAARDFINWLIQTKITK,
SEQ ID NO: 19
H-Aib-DGTFISDYST-Aib-LDKLAARDFIEWLIQTKITK,
SEQ ID NO: 20
H-Aib-DGTFISDYST-Aib-LDKLAARDFINWLIETKITK,
SEQ ID NO: 34
H-Aib-DGTFISDYST-Aib-
LDNLAARDFINWLIQTKITKNDWKHNITQ,
SEQ ID NO: 35
H-Aib-EGTFISDYST-Aib-
LDALAARDFINWLIQTKITKNDWKHNITQ,
SEQ ID NO: 36
H-Aib-DGTFISDYST-Aib-
LDNLAAKDFINWLIQTKITKNDWKHNITQ,
SEQ ID NO: 37
H-Aib-EGTFISDYST-Aib-
LDNLAAKDFINWLIQTKITKNDWKHNITQ,
and
SEQ ID NO: 4
H-Aib-DGTFISDYST-Aib-LDNLAARDFINWLIQTKITK

or a functional variant thereof, wherein said functional variant comprises 1 to 3 individual amino acid substitutions with the proviso that the amino acid residue at position 2 of said functional variant is Aib,
the amino acid residue at position 6 of said functional variant is Phenylalanine (F),
the amino acid residue at position 22 of said functional variant is Phenylalanine (F),
the amino acid residue at position 25 of said functional variant is Tryptophan (W), and
the amino acid residue at position 26 of said functional variant is Leucine (L),
wherein said peptide is modified by attaching at least one fatty acid molecule at one or more amino acid residues at any one of positions 1 to 15 of any one of the above sequences, and
wherein said peptide is an agonist of GIPR (glucose-dependent insulinotropic polypeptide receptor) and is an agonist of GLP-2R (glucagon-like peptide-2 receptor).

In one aspect, the present disclosure relates to a peptide dual agonist comprising or consisting of an amino acid sequence selected from the group consisting of:

SEQ ID NO: 1
(C16)HADGTFISDYSTILDNLAARDFINWLIQTKITD
SEQ ID NO: 2
(C16)HADGTFISDYSTILDNLAARDFINWLIQTKIT
SEQ ID NO: 38
(C16)HADGTFISDYSTILDNLAAKDFINWLIQTKITK
SEQ ID NO: 3
(C10)HADGTFISDYSTILDNLAAKDFINWLIQTKITKNDWKHNITQ
SEQ ID NO: 39
(C16)HADGTFISDYSTILDNLAARDFINWLLAQKITK
SEQ ID NO: 1
(C16)HADGTFISDYSTILDNLAARDFINWLIQTKITD
SEQ ID NO: 4
(C16)H-Aib-DGTFISDYST-Aib-LDNLAARDFINWLIQTKITK-OH
SEQ ID NO: 5
(C16)H-Aib-DGTFISDYSTILD-Aib-LAARDFINWLIQTKITK-OH
SEQ ID NO: 6
(C16)H-Aib-DGTFISDYSTILDNLAARDFIEWLIQTKITK-OH
SEQ ID NO: 7
(C16)H-Aib-DGTFISDYSTILDNLAARDFIEWLIQTKIT-OH
SEQ ID NO: 14
(C16)HADGTFISDYSTILELLAARDFINWLIQTKITKNDWKHNITQ
SEQ ID NO: 41
(C16)HADGTFISDYSTILDELAARDFINWLIQTKITKNDWKHNITQ
SEQ ID NO: 42
(C16)HADGTFISDYSTILDNLAARDFINWLIQTKITKAEWKHAITQ
SEQ ID NO: 43
(C16)HADGTFISDYSTILDNLAARDFIEWLIQTKITKNDWKHNITQ
SEQ ID NO: 44
(C16)HADGTFISDYSTILDNLAARDFINWLIETKITKNDWKHNITQ
SEQ ID NO: 45
(C16)HADGTFISDYSTILDNLAARDFINWLIATKITKNDWKHNITQ
SEQ ID NO: 33
(C16)HADGTFISDYSTAibLDNLAARDFINWLIQTKITKNDWKHNITQ
SEQ ID NO: 15
(C16)HADGTFISDYSTILDAibLAARDFINWLIQTKITKNDWKHNITQ
SEQ ID NO: 4
(C16-diacid)H-Aib-DGTFISDYST-Aib-
LDNLAARDFINWLIQTKITK-OH
SEQ ID NO: 4
(C10-diacid)H-Aib-DGTFISDYST-Aib-
LDNLAARDFINWLIQTKITK-OH
SEQ ID NO: 16
(C16-diacid)H-Aib-EGTFISDYST-Aib-
LDNLAARDFINWLIQTKITK-OH
SEQ ID NO: 17
(C16-diacid)H-Aib-DGTFISDYST-Aib-
LDALAARDFIEWLIQTKITK-OH
SEQ ID NO: 18
(C16-diacid)H-Aib-EGTFISDYST-Aib-
LDKLAARDFINWLIQTKITK-OH
SEQ ID NO: 19
(C16-diacid)H-Aib-DGTFISDYST-Aib-
LDKLAARDFIEWLIQTKITK-OH
SEQ ID NO: 20
(C16-diacid)H-Aib-DGTFISDYST-Aib-
LDKLAARDFINWLIETKITK-OH
SEQ ID NO: 33
(C16-diacid)HADGTFISDYSTAibLDNLAARDFINWLIQTKITKNDW
KHNITQ
SEQ ID NO: 34
(C16-diacid)H-Aib-DGTFISDYST-Aib-
LDNLAARDFINWLIQTKITKNDWKHNITQ-OH
SEQ ID NO: 35
(C16-diacid)H-Aib-EGTFISDYST-Aib-
LDALAARDFINWLIQTKITKNDWKHNITQ-OH
SEQ ID NO: 36
(C16-diacid)H-Aib-DGTFISDYST-Aib-
LDNLAAKDFINWLIQTKITKNDWKHNITQ-OH
SEQ ID NO: 37
(C16-diacid)H-Aib-EGTFISDYST-Aib-
LDNLAAKDFINWLIQTKITKNDWKHNITQ-OH,
and
SEQ ID NO: 4
(C10)H-Aib-DGTFISDYST-Aib-LDNLAARDFINWLIQTKITK-OH,

or a functional variant thereof, wherein said functional variant comprises 1 to 3 individual amino acid substitutions with the proviso that the amino acid residue at position 2 of said functional variant is Aib,
the amino acid residue at position 6 of said functional variant is Phenylalanine (F),
the amino acid residue at position 22 of said functional variant is Phenylalanine (F),
the amino acid residue at position 25 of said functional variant is Tryptophan (W), and
the amino acid residue at position 26 of said functional variant is Leucine (L),
wherein said peptide is modified by attaching at least one fatty acid molecule at position 1 of any one of the listed amino acid sequences, and
wherein said peptide is an agonist of GIPR (glucose-dependent insulinotropic polypeptide receptor) and is an agonist of GLP-2R (glucagon-like peptide-2 receptor).

In one embodiment the peptide dual agonist of the present disclosure is C-terminally amidated (—NH₂).

In one embodiment the peptide dual agonist of the present disclosure, the C-terminus of the peptide is a carboxylic acid.

In one embodiment the peptide dual agonist of the present disclosure is N-terminally acetylated (COCH₃).

In one embodiment the peptide dual agonist of the present disclosure is Compound no. 24 H-Aib-DG-K(γGlu-C10)-FISDYST-Aib-LDNLAARDFINWLIQTKITK-OH (SEQ ID NO: 21), or a functional variant or a functional fragment thereof. This peptide dual agonist is particularly potent and selective for the GIPR and the GLP-1R.

In one embodiment the peptide dual agonist of the present disclosure is Compound no. 25 H-Aib-DG-K(γGlu-C16 diacid)-FISDYST-Aib-LDNLAARDFINWLIQTKITK-OH (SEQ ID NO: 21), or a functional variant or a functional fragment thereof. This peptide dual agonist is particularly potent and selective for the GIPR and the GLP-1R.

In one embodiment the peptide dual agonist of the present disclosure is Compound no. 31 H-Aib-DGTFISDY-K(γGlu-C10)-TILDKLAARDFIEWLIATKITK-OH (SEQ ID NO: 26), or a functional variant or a functional fragment thereof. This peptide dual agonist is particularly potent and selective for the GIPR and the GLP-1R.

In one embodiment the peptide dual agonist of the present disclosure is Compound no. 33 H-Aib-DGTFISDY-K(γGlu-C16)-T-Aib-LDALAARDFIEWLIQTKITK-OH (SEQ ID NO: 28), or a functional variant or a functional fragment thereof. This peptide dual agonist is particularly potent and selective for the GIPR and the GLP-1R.

In one embodiment the peptide dual agonist of the present disclosure is Compound no. 37 H-Aib-DGTFISDY-K(γGlu-16)-T-Aib-LDNLAARDFINWLIQTKITKNDWKHNITQ-OH (SEQ ID NO: 31), or a functional variant or a functional fragment thereof. This peptide dual agonist is particularly potent and selective for the GIPR and the GLP-1R.

In one embodiment the peptide dual agonist of the present disclosure is Compound no. 40 (C16-diacid) H-Aib-DGTFISDYST-Aib-LDNLAARDFINWLIQTKITKNDWKHNITQ-OH (SEQ ID NO: 34), or a functional variant or a functional fragment thereof. This peptide dual agonist is particularly potent and selective for the GIPR and the GLP-1R.

A peptide that comprises or consists of a sequence means that the peptide can comprise the sequence, consist of the sequence, or comprise at least the full sequence. A peptide that comprises a peptide sequence, such as comprising the sequence H-Aib-DGTFISDYST-Aib-LDNLAARDFINWLIQTKITKNDWKHNITQ (SEQ ID NO:) 34, means that the peptide includes all of the peptide sequence H-Aib-DGTFISDYST-Aib-LDNLAARDFINWLIQTKITKNDWKHNITQ (SEQ ID NO: 34). It does, however, not exclude that additional components or amino acids are present.

In one embodiment a functional variant of a peptide dual agonist has at least 60% sequence identity, such as at least 70% sequence identity, such as at least 75% sequence identity, such as at least 80% sequence identity, such as at least 85% sequence identity, such as at least 90% sequence identity, such as at least 95% sequence identity, such as at least 97% sequence identity to said peptide dual agonist.

In one embodiment a functional variant of a peptide dual agonist has 60 to 65% sequence identity, such as 65 to 70% sequence identity, such as 70 to 75% sequence identity, such as 75 to 80% sequence identity, such as 80 to 85% sequence identity, such as 85 to 90% sequence identity, such as 90 to 95% sequence identity, such as 95 to 99% sequence identity, such as 99 to 100% sequence identity to said peptide dual agonist. ‘Identity’ and ‘sequence identity’ may be used interchangeably herein.

In one embodiment a functional variant comprises one or more amino acid substitutions, such as 1 to 8 amino acid substitutions, such as 1 to 2, 2 to 3, 3 to 4, 4 to 5, 5 to 6, 6 to 7 or 7 to 8 amino acid substitutions.

In one embodiment a functional variant comprises one amino acid substitution, two amino acid substitutions, three amino acid substitutions, four amino acid substitutions or five amino acid substitutions.

In one embodiment said amino acid substitutions are conservative amino acid substitutions. In one embodiment said functional variant comprises one or more conservative amino acid substitutions.

A conservative substitution (or synonymous substitution) is the substitution of amino acids whose side chains have similar biochemical properties and thus do not affect the function of the peptide.

The identity between amino acid sequences may be calculated using well known algorithms such as BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, or BLOSUM 90, or by simple comparison of the specific amino acids present at corresponding positions in two peptide sequences to be compared. Homology may be used as a synonym to identity/sequence identity.

Conservative substitutions may be introduced in any one or more positions of a peptide according to the present disclosure, as long as the variant remains functional. It may however also be desirable to introduce non-conservative substitutions in one or more positions (non-synonymous substitutions).

Any amino acids as defined herein may be in the L- or D-configuration. If nothing is specified, reference to the L-isomeric form is preferably meant.

The standard and/or non-standard amino acids may be linked by peptide bonds (to form a linear peptide chain), or by non-peptide bonds (e.g. via the variable side-chains of the amino acids). Preferably, the amino acids of the present disclosure are linked by peptide bonds.

The terms ‘peptide’ and ‘isolated peptide’ may be used interchangeably herein. The terms ‘variant’ and ‘functional variant’ may be used interchangeably herein. The terms ‘fragment’ and ‘functional fragment’ may be used interchangeably herein. When reference is made to a ‘peptide’ herewith, this term will encompass both references to a peptide per se, and also to a peptide for use as defined herein.

In one embodiment the peptide is non-naturally occurring.

In one embodiment the peptide is synthetic.

In one embodiment the peptide is an isolated peptide.

In one embodiment the peptide is a labeled peptide, such as radiolabeled or fluorescent labeled peptide.

In another embodiment, a variant as defined herein includes sequences wherein an alkyl amino acid is substituted for an alkyl amino acid, wherein an aromatic amino acid is substituted for an aromatic amino acid, wherein a sulfur-containing amino acid is substituted for a sulfur-containing amino acid, wherein a hydroxy-containing amino acid is substituted for a hydroxy-containing amino acid, wherein an acidic amino acid is substituted for an acidic amino acid, wherein a basic amino acid is substituted for a basic amino acid, and/or wherein a dibasic monocarboxylic amino acid is substituted for a dibasic monocarboxylic amino acid.

The term peptide also embraces post-translational modifications introduced by chemical or enzyme-catalyzed reactions, as are known in the art. These include acetylation, phosphorylation, methylation, glucosylation, glycation, amidation, hydroxylation, deimination, deamidation, carbamylation and sulfation of one or more amino acid residues, and also proteolytic modification by known proteinases including lysosomal kathepsins, and also calpains, secretases and matrix-metalloproteinases.

Also, functional equivalents of the peptides may comprise chemical modifications such as ubiquitination, labeling (e.g., with radionuclides, various enzymes, etc.), pegylation (derivatization with polyethylene glycol), or by insertion (or substitution by chemical synthesis) of amino acids such as ornithine, which do not normally occur in human proteins (non-proteinogenic).

Sterically similar compounds may be formulated to mimic the key portions of the peptide structure. This may be achieved by techniques of modelling and chemical designing known to those of skill in the art. For example, esterification and other alkylations may be employed to modify the amino terminus of e.g. a di-arginine peptide backbone, to mimic a tetra peptide structure. It will be understood that all such sterically similar constructs fall within the scope of the present invention. Peptides with N-terminal and C-terminal alkylations and esterifications are also encompassed within the present invention.

A contiguous or consecutive peptide sequence is a sequence of consecutive amino acids being linked linearly by peptide bonds. Contiguous and consecutive amino acid sequence is used interchangeably herein.

### Attachment of at Least One Fatty Acid Molecule

The peptide dual agonists of the present disclosure are characterized by being modified by attachment of at least one fatty acid molecule at an amino acid residue at any one of positions 1 to 15 of the disclosed amino acid sequences. In fact, attachment of a fatty acid on the N-terminal region of the disclosed amino acid sequences, such as at any one of positions 1 to 15 of the disclosed sequences, results in a dual peptide agonist of hGIPR and hGLP-2R, which has low or no ability to activate the hGLP-1R and is therefore referred to as a “selective peptide dual agonist of hGIPR and hGLP-2R”.

In one embodiment the peptide dual agonist of the present disclosure is modified by attaching at least one fatty acid molecule at position 1, or at the N-terminus, of the disclosed amino acid sequences.

In one embodiment the peptide dual agonist according to the present disclosure, comprises or consists of SEQ ID NO: 66, 67, 68 or 69, wherein X₇ is I, and wherein said peptide is modified by attaching one fatty acid molecule at position 1 of SEQ ID NO: 66, 67, 68 or 69, such as at the N-terminus of SEQ ID NO: 66, 67, 68 or 69.

In one embodiment the fatty acid molecule according to the present disclosure is attached to the alpha-amino group of an amino acid residue via a linker or spacer, wherein said amino acid residue is the N-terminal amino acid residue.

The peptide dual agonists of the present disclosure are characterized by being modified by attachment of at least one fatty acid molecule at an amino acid residue at any one of positions 2 to 15 of the disclosed amino acid sequences.

The peptide dual agonists of the present disclosure are characterized by being modified by attachment of at least one fatty acid molecule at an amino acid residue at any one of positions 2 to 14 of the disclosed amino acid sequences.

In one embodiment the peptide dual agonist of the present disclosure is modified by attaching at least one fatty acid molecule at a Lysine (K) at any one of positions 2 to 15 of SEQ ID NO: 66, 67, 68 or 69.

In one embodiment the peptide dual agonist of the present disclosure is modified by attaching at least one fatty acid molecule at position 2, position 3, position 4, position 5, position 6, position 7, position 8, position 9, position 10, position 11, position 12, position 13, position 14, or position 15 of SEQ ID NO: 66, 67, 68 or 69, or a variant thereof disclosed herein.

In one embodiment the peptide dual agonist of the present disclosure is modified by attaching at least one fatty acid molecule at position 2, position 3, position 4, position 5, position 6, position 7, position 8, position 9, position 10, position 11 or position 12 of SEQ ID NO: 66, 67, 68 or 69, or a variant thereof disclosed herein.

In one embodiment the peptide dual agonist of the present disclosure is modified by attaching at least one fatty acid molecule at position 2, position 3, position 4, position 5, position 6, position 7, position 8, position 9, position 10 or position 11 of SEQ ID NO: 66, 67, 68 or 69, or a variant thereof disclosed herein.

In one embodiment the peptide dual agonist of the present disclosure is modified by attaching at least one fatty acid molecule at position 5, position 6, position 7, position 8, position 9, position 10, position 11, position 12, position 13, position 14, or position 15 of SEQ ID NO: 66, 67, 68 or 69, or a variant thereof disclosed herein.

In one embodiment the peptide dual agonist of the present disclosure is modified by attaching at least one fatty acid molecule at position 5, position 6, position 7, position 8, position 9, position 10, position 11 or position 12 of SEQ ID NO: 66, 67, 68 or 69, or a variant thereof disclosed herein.

In one embodiment the peptide dual agonist of the present disclosure is modified by attaching at least one fatty acid molecule at any one of positions 5, 7, 8, 11, 12, 15 of SEQ ID NO: 66, 67, 68 or 69, or a variant thereof disclosed herein.

In one embodiment the peptide dual agonist of the present disclosure is modified by attaching at least one fatty acid molecule at any one of positions 5, 7, 8, 11 of SEQ ID NO: 66, 67, 68 or 69, or a variant thereof disclosed herein.

In one embodiment the peptide dual agonist of the present disclosure is modified by attaching at least one fatty acid molecule at any one of positions 5, 11, 12 of SEQ ID NO: 66, 67, 68 or 69, or a variant thereof disclosed herein.

It has been found that attachment of a fatty acid at N-terminal regions, such as at positions 1 to 15 of the disclosed peptides, such as at the N-terminus, or positions 5, 11 and/or 12 reduces activation of GLP-1R. Instead, attachment of a fatty acid at the C-terminal region such as at positions 16 to 43 of a peptide dual agonist of GIPR and GLP-2R may cause retention of GLP-1R agonist activity and may also increase potency towards GLP-1R.

In one embodiment the peptide dual agonist of the present disclosure comprises or consists of the amino acid sequence SEQ ID NO: 66, 67, 68 or 69 wherein X₁₃ is Aib, and said peptide is modified by attaching at least one fatty acid molecule at any one of positions 5 or 11 of SEQ ID NO: 66, 67, 68 or 69.

In one embodiment of the present disclosure, the fatty acid molecule is a straight-chain fatty acid.

In one embodiment of the present disclosure, said fatty acid molecule is a branched fatty acid.

In one embodiment of the present disclosure, said fatty acid molecule is a monoacyl fatty acid molecule, comprising one fatty acid.

In one embodiment of the present disclosure, said fatty acid molecule is a diacyl fatty acid molecule.

In one embodiment of the present disclosure, said fatty acid molecule comprises an acyl group selected from the group consisting of CH₃(CH₂)₈CO— (capriyl, C10), CH₃(CH₂)₁₀CO— (lauryl, C12), CH₃(CH₂)₁₂CO— (myristoyl, C14), CH₃(CH₂)₁₄CO— (palmitoyl, C16), CH₃(CH₂)₁₆CO— (stearyl, C18) and CH₃(CH₂)₁₈CO— (arachidyl, C20).

In one embodiment of the present disclosure, said fatty acid molecule comprises two acyl groups individually selected from the group consisting of HOOC—CH₃(CH₂)₈CO— (decanoyl, C10), HOOC—CH₃(CH₂)₁₀CO— (dodecanoyl, C12), HOOC—CH₃(CH₂)₁₂CO— (1-tetradecanoyl, C14), HOOC—CH₃(CH₂)₁₄CO— (hexadecanoyl, C16), HOOC—CH₃(CH₂)₁₅CO— (15-carboxy-pentadecanoyl, C17), HOOC—CH₃(CH₂)₁₆CO— (octadecanoyl, C18), HOOC—CH₃(CH₂)₁₇CO— (17-carboxy-heptadecanoyl, C19), HOOC—CH₃(CH₂)₁₈CO— (eicosanoyl, C20), and HOOC—CH₃(CH₂)₁₉CO— (19-carboxy-nonadecanoyl, C21).

In one embodiment said fatty acid molecule is C16 (palmitoyl).

In one embodiment said fatty acid molecule comprises an acyl group selected from the group consisting of COOH(CH₂)₈CO— (C10 diacid), COOH(CH₂)₁₀CO— (C12 diacid), COOH(CH₂)₁₂CO— (C14 diacid), COOH(CH₂)₁₄CO— (C16 diacid), COOH(CH₂)₁₆CO— (C18 diacid), COOH(CH₂)₁₈CO— (C20 diacid) and COOH(CH₂)₂₀CO— (C22 diacid).

In one embodiment of the present disclosure, said fatty acid molecule is attached to an amino acid residue directly, such as without the presence of a spacer or linker.

In one embodiment of the present disclosure, said fatty acid molecule is attached to an amino acid residue via a spacer or linker.

In one embodiment the spacer is a hydrophilic linker. In one embodiment the spacer is a non-natural amino acid hydrophilic linker.

In one embodiment the spacer is a repeat of individual spacer moieties. In one embodiment the spacer is a repeat of identical spacer moieties. In one embodiment the spacer is a repeat of different spacer moieties.

In one embodiment, the fatty acid molecule is attached to an amino acid residue via a spacer in such a way that a carboxyl group of the spacer forms an amide bond with an amino group of the fatty acid molecule.

In one embodiment the spacer comprises one or more moieties individually selected from the group consisting of:
one or more amino acids selected from the group consisting of succinic acid, Lys, Glu, Asp,
4-Abu,
y-aminobuturic acid,
one or more of γ-aminobutanoyl (γ-aminobutyric acid), γ-glutamyl (γ-glutamic acid), β-asparagyl, β-alanyl and glycyl, and
[γ-glutamic acid-8-amino-3,6-dioxaoctanoic acid]ₙ (γGlu-AEEAcₙ,), wherein n is an integer between 1 and 50, such as an integer between 1-2, 2-3, 3-4, 4-5, 5-6, 6-7, 7-8, 8-9, 9-10, 10-11, 11-12, 12-13, 13-14, 14-15, 15-20, 20-25, 25-30, 30-35, 35-40, 40-45, 45-50.

In one embodiment of the present disclosure, the spacer or linker comprises γGlu.

In one embodiment of the present disclosure, said spacer comprises or consists of γGlu or γGlu-γGlu.

In some embodiments of the present disclosure the peptide dual agonists disclosed herein are acylated, which in some embodiments increase half-life and in vivo stability, in addition to rendering the peptide dual agonists selective agonists of GIPR and GLP-2R, and not GLP-1R, and retaining their agonistic potency.

In one embodiment the peptide dual agonist of the present disclosure is selected from the group consisting of

Compound no. 9
SEQ ID NO: 8
H-Aib-DG-K(γGlu-C16)-FISDYSTILDNLAARDFINWLIQTKITK-
OH
Compound no. 10
SEQ ID NO: 9
H-Aib-DGTF-K(γGlu-C16)-SDYSTILDNLAARDFINWLIQTKITK-
OH
Compound no. 11
SEQ ID NO: 10
H-Aib-DGTFISDY-K(γGlu-C16)-TILDNLAARDFINWLIQTKITK-
OH
Compound no. 12
SEQ ID NO: 11
H-Aib-DGTFISDYS-K(γGlu-C16)-ILDNLAARDFINWLIQTKITK-
OH
Compound no. 13
SEQ ID NO: 12
H-Aib-DGTFISDYSTIL-K(γGlu-C16)-NLAARDFINWLIQTKITK-
OH
Compound no. 14
SEQ ID NO: 13
H-Aib-DGTFISDY-K(γGlu-C16)-
TILDNLAARDFINWLIQTKGKKNDWKHNITQ-NH₂
Compound no. 24
SEQ ID NO: 21
H-Aib-DG-K(γGlu-C10)-FISDYST-Aib-
LDNLAARDFINWLIQTKITK-OH
Compound no. 25
SEQ ID NO: 21
H-Aib-DG-K(γGlu-C16 diacid)-FISDYST-Aib-
LDNLAARDFINWLIQTKITK-OH
Compound no. 26
SEQ ID NO: 8
H-Aib-DG-K(γGlu- γGlu-C10)-
FISDYSTILDNLAARDFINWLIQTKITK-OH
Compound no. 27
SEQ ID NO: 22
H-Aib-EG-K(γGlu-C16)-FISDYST-Aib-
LDNLAARDFINWLIQTKITK-OH
Compound no. 28
SEQ ID NO: 21
H-Aib-DG-K(γGlu- γGlu-C16)-FISDYST-Aib-
LDNLAARDFINWLIQTKITK-OH
Compound no. 29
SEQ ID NO: 23
H-Aib-DGTFISDY-K(γGlu-C16)-T-Aib-
LDNLAARDFINWLIQTKITK-OH
Compound no. 30
SEQ ID NO: 25
H-Aib-DGTFISDY-K(γGlu-C10)-T-Aib-
LDNLAARDFINWLIQTKITD-OH
Compound no. 31
SEQ ID NO: 26
H-Aib-DGTFISDY-K(γGlu-C10)-TILDKLAARDFIEWLIATKITK-
OH
Compound no. 32
SEQ ID NO: 27
H-Aib-DGTFISDY-K(γGlu-C16)-TILDNLAARDFINWLIETKITK-
OH
Compound no. 33
SEQ ID NO: 28
H-Aib-DGTFISDY-K(γGlu-C16)-T-Aib-
LDALAARDFIEWLIQTKITK-OH
Compound no. 34
SEQ ID NO: 24
Ac-H-Aib-DGTFISDY-K(γGlu-C16)-T-Aib-
LDNLAARDFINWLIQTKITK-OH
Compound no. 35
SEQ ID NO: 29
H-Aib-DGTFISDYS-K(γGlu-C16)-Aib-
LDNLAARDFINWLIQTKITK-OH
Compound no. 36
SEQ ID NO: 30
Ac-H-Aib-DGTFISDYS-K(γGlu-C16)-Aib-
LDNLAARDFINWLIQTKITK-OH
Compound no. 37
SEQ ID NO: 31
H-Aib-DGTFISDY-K(γGlu-C16)-TAib-
LDNLAARDFINWLIQTKITKNDWKHNITQ-OH
Compound no. 38
SEQ ID NO: 32
Ac-H-Aib-DGTFISDY-K(γGlu-C16)-T-Aib-
LDNLAARDFINWLIQTKITKNDWKHNITQ-OH
Compound no. 54
SEQ ID NO: 46
H-Aib-DGTFISDY-K(yglu-C10)-T-Aib-
LDNLAARDFINWLIQTKITGNDWKHNITQ-OH
Compound no. 1
SEQ ID NO: 1
(C16)HADGTFISDYSTILDNLAARDFINWLIQTKITD
Compound no. 2
SEQ ID NO: 2
(C16)HADGTFISDYSTILDNLAARDFINWLIQTKIT
Compound no. 45
SEQ ID NO: 38
(C16)HADGTFISDYSTILDNLAAKDFINWLIQTKITK
Compound no. 3
SEQ ID NO: 3
(C10)HADGTFISDYSTILDNLAAKDFINWLIQTKITKNDWKHNITQ
Compound no. 46
SEQ ID NO: 39
(C16)HADGTFISDYSTILDNLAARDFINWLLAQKITK
Compound no. 4
SEQ ID NO: 1
(C16)HADGTFISDYSTILDNLAARDFINWLIQTKITD
Compound no. 5
SEQ ID NO: 4
(C16)H-Aib-DGTFISDYST-Aib-LDNLAARDFINWLIQTKITK-OH
Compound no. 6
SEQ ID NO: 5
(C16)H-Aib-DGTFISDYSTILD-Aib-LAARDFINWLIQTKITK-OH
Compound no. 7
SEQ ID NO: 6
(C16)H-Aib-DGTFISDYSTILDNLAARDFIEWLIQTKITK-OH
Compound no. 8
SEQ ID NO: 7
(C16)H-Aib-DGTFISDYSTILDNLAARDFIEWLIQTKIT-OH
Compound no. 15
SEQ ID NO: 14
(C16)HADGTFISDYSTILELLAARDFINWLIQTKITKNDWKHNITQ
Compound no. 48
SEQ ID NO: 41
(C16)HADGTFISDYSTILDELAARDFINWLIQTKITKNDWKHNITQ
Compound no. 49
SEQ ID NO: 42
(C16)HADGTFISDYSTILDNLAARDFINWLIQTKITKAEWKHAITQ
Compound no. 50
SEQ ID NO: 43
(C16)HADGTFISDYSTILDNLAARDFIEWLIQTKITKNDWKHNITQ
Compound no. 51
SEQ ID NO: 44
(C16)HADGTFISDYSTILDNLAARDFINWLIETKITKNDWKHNITQ
Compound no. 52
SEQ ID NO: 45
(C16)HADGTFISDYSTILDNLAARDFINWLIATKITKNDWKHNITQ
Compound no. 53
SEQ ID NO: 33
(C16)HADGTFISDYSTAibLDNLAARDFINWLIQTKITKNDWKHNITQ
Compound no. 16
SEQ ID NO: 15
(C16)HADGTFISDYSTILDAibLAARDFINWLIQTKITKNDWKHNITQ
Compound no. 17
SEQ ID NO: 4
(C16-diacid)H-Aib-DGTFISDYST-Aib-
LDNLAARDFINWLIQTKITK-OH
Compound no. 18
SEQ ID NO: 4
(C10-diacid)H-Aib-DGTFISDYST-Aib-
LDNLAARDFINWLIQTKITK-OH
Compound no. 19
SEQ ID NO: 16
(C16-diacid)H-Aib-EGTFISDYST-Aib-
LDNLAARDFINWLIQTKITK-OH
Compound no. 20
SEQ ID NO: 17
(C16-diacid)H-Aib-DGTFISDYST-Aib-
LDALAARDFIEWLIQTKITK-OH
Compound no. 21
SEQ ID NO: 18
(C16-diacid)H-Aib-EGTFISDYST-Aib-
LDKLAARDFINWLIQTKITK-OH
Compound no. 22
SEQ ID NO: 19
(C16-diacid)H-Aib-DGTFISDYST-Aib-
LDKLAARDFIEWLIQTKITK-OH
Compound no. 23
SEQ ID NO: 20
(C16-diacid)H-Aib-DGTFISDYST-Aib-
LDKLAARDFINWLIETKITK-OH
Compound no. 39
SEQ ID NO: 33
(C16-diacid)HADGTFISDYSTAibLDNLAARDFINWLIQTKITKND
WKHNITQ
Compound no. 40
SEQ ID NO: 34
(C16-diacid)H-Aib-DGTFISDYST-Aib-
LDNLAARDFINWLIQTKITKNDWKHNITQ-OH
Compound no. 41
SEQ ID NO: 35
(C16-diacid)H-Aib-EGTFISDYST-Aib-
LDALAARDFINWLIQTKITKNDWKHNITQ-OH
Compound no. 42
SEQ ID NO: 36
(C16-diacid)H-Aib-DGTFISDYST-Aib-
LDNLAAKDFINWLIQTKITKNDWKHNITQ-OH
Compound no. 43
SEQ ID NO: 37
(C16-diacid)H-Aib-EGTFISDYST-Aib-
LDNLAAKDFINWLIQTKITKNDWKHNITQ-OH
Compound no. 44
SEQ ID NO: 4
(C10)H-Aib-DGTFISDYST-Aib-LDNLAARDFINWLIQTKITK-OH,

or a variant thereof, wherein said variant thereof comprises 1 or 2 individual amino acid substitutions.

In one embodiment the peptide dual agonist is selected from the group consisting of

SEQ ID NO: 4
(C16)H-Aib-DGTFISDYST-Aib-LDNLAARDFINWLIQTKITK-OH,
SEQ ID NO: 5
(C16)H-Aib-DGTFISDYSTILD-Aib-LAARDFINWLIQTKITK-OH,
Compound no. 9
SEQ ID NO: 8
H-Aib-DG-K(γGlu-C16)-FISDYSTILDNLAARDFINWLIQTKITK-
OH,
Compound no. 10
SEQ ID NO: 10
H-Aib-DGTFISDY-K(γGlu-C16)-TILDNLAARDFINWLIQTKITK-
OH,
SEQ ID NO: 11
H-Aib-DGTFISDYS-K(γGlu-C16)-ILDNLAARDFINWLIQTKITK-
OH,
SEQ ID NO: 13
H-Aib-DGTFISDY-K(γGlu-C16)-
TILDNLAARDFINWLIQTKGKKNDWKHNITQ-NH2,
SEQ ID NO: 4
(C16-diacid)H-Aib-DGTFISDYST-Aib-
LDNLAARDFINWLIQTKITK-OH,
SEQ ID NO: 16
(C16-diacid)H-Aib-EGTFISDYST-Aib-
LDNLAARDFINWLIQTKITK-OH,
SEQ ID NO: 17
(C16-diacid)H-Aib-DGTFISDYST-Aib-
LDALAARDFIEWLIQTKITK-OH,
SEQ ID NO: 19
(C16-diacid)H-Aib-DGTFISDYST-Aib-
LDKLAARDFIEWLIQTKITK-OH,
SEQ ID NO: 21
H-Aib-DG-K(γGlu-C10)-FISDYST-Aib-
LDNLAARDFINWLIQTKITK-OH,
SEQ ID NO: 21
H-Aib-DG-K(γGlu-C16 diacid)-FISDYST-Aib-
LDNLAARDFINWLIQTKITK-OH,
SEQ ID NO: 21
H-Aib-DGTFISDY-K(γGlu-C16)-T-Aib-
LDNLAARDFINWLIQTKITK-OH,
SEQ ID NO: 28
H-Aib-DGTFISDY-K(γGlu-C16)-T-Aib-
LDALAARDFIEWLIQTKITK-OH,
SEQ ID NO: 31
H-Aib-DGTFISDY-K(γGlu-C16)-TAib-
LDNLAARDFINWLIQTKITKNDWKHNITQ-OH,
SEQ ID NO: 34
(C16-diacid)H-Aib-DGTFISDYST-Aib-
LDNLAARDFINWLIQTKITKNDWKHNITQ-OH,
SEQ ID NO: 35
(C16-diacid)H-Aib-EGTFISDYST-Aib-
LDALAARDFINWLIQTKITKNDWKHNITQ-OH,
SEQ ID NO: 36
(C16-diacid)H-Aib-DGTFISDYST-Aib-
LDNLAAKDFINWLIQTKITKNDWKHNITQ-OH,
SEQ ID NO: 4
(C10)H-Aib-DGTFISDYST-Aib-LDNLAARDFINWLIQTKITK-OH,
and
SEQ ID NO: 26
H-Aib-DGTFISDY-K(γGlu-C10)-TILDKLAARDFIEWLIATKITK-
OH,

or a variant thereof, wherein said variant thereof comprises 1 or 2 individual amino acid substitutions.

In one embodiment the peptide dual agonist comprises or consists of the sequence

Compound no. 5
SEQ ID NO: 4
(C16)H-Aib-DGTFISDYST-Aib-LDNLAARDFINWLIQTKITK-OH.

In one embodiment the peptide dual agonist comprises or consists of the sequence

Compound no. 6
SEQ ID NO: 5
(C16)H-Aib-DGTFISDYSTILD-Aib-LAARDFINWLIQTKITK-OH.

In one embodiment the peptide dual agonist comprises or consists of the sequence

Compound no. 9
SEQ ID NO: 8
H-Aib-DG-K(γGlu-C16)-FISDYSTILDNLAARDFINWLIQTKITK-
OH.

In one embodiment the peptide dual agonist comprises or consists of the sequence

SEQ ID NO: 10
Compound no. 11 H-Aib-DGTFISDY-K(γGlu-C16)-
TILDNLAARDFINWLIQTKITK-OH.

In one embodiment the peptide dual agonist comprises or consists of the sequence

Compound no. 12 SEQ ID NO: 11
H-Aib-DGTFISDYS-K(γGlu-C16)-ILDNLAARDFINWLIQTKITK-OH.

In one embodiment the peptide dual agonist comprises or consists of the sequence

Compound no. 14 SEQ ID NO: 13
H-Aib-DGTFISDY-K(γGlu-C16)-TILDNLAARDFINWLIQTKGKKNDWKHNITQ-NH2.

In one embodiment the peptide dual agonist comprises or consists of the sequence

Compound no. 17 SEQ ID NO: 4
(C16-diacid)H-Aib-DGTFISDYST-Aib-LDNLAARDFINWLIQTKITK-OH.

In one embodiment the peptide dual agonist comprises or consists of the sequence

Compound no. 19 SEQ ID NO: 16
(C16-diacid)H-Aib-EGTFISDYST-Aib-LDNLAARDFINWLIQTKITK-OH.

In one embodiment the peptide dual agonist comprises or consists of the sequence

Compound no. 20 SEQ ID NO: 17
(C16-diacid)H-Aib-DGTFISDYST-Aib-LDALAARDFIEWLIQTKITK-OH.

In one embodiment the peptide dual agonist comprises or consists of the sequence

Compound no. 22 SEQ ID NO: 19
(C16-diacid)H-Aib-DGTFISDYST-Aib-LDKLAARDFIEWLIQTKITK-OH.

In one embodiment the peptide dual agonist comprises or consists of the sequence Compound no. 24 SEQ ID NO: 21 H-Aib-DG-K(γGlu-C10)-FISDYST-Aib-LDNLAARDFINWLIQTKITK-OH. This peptide dual agonist has both good potency and good physical stability.

In one embodiment the peptide dual agonist comprises or consists of the sequence Compound no. 25 SEQ ID NO: 21 H-Aib-DG-K(γGlu-C16 diacid)-FISDYST-Aib-LDNLAARDFINWLIQTKITK-OH. This peptide dual agonist has both good potency and good physical stability.

In one embodiment the peptide dual agonist comprises or consists of the sequence

Compound no. 25 SEQ ID NO: 21
H-Aib-DGTFISDY-K(γGlu-C16)-T-Aib-LDNLAARDFINWLIQTKITK-OH.

In one embodiment the peptide dual agonist comprises or consists of the sequence Compound no. 33 SEQ ID NO: 28 H-Aib-DGTFISDY-K(γGlu-C16)-T-Aib-LDALAARDFIEWLIQTKITK-OH. This peptide dual agonist has both good potency and good physical stability.

In one embodiment the peptide dual agonist comprises or consists of the sequence

Compound no. 35 SEQ ID NO: 29
H-Aib-DGTFISDYS-K(γGlu-C16)-Aib-LDNLAARDFINWLIQTKITK-OH.

In one embodiment the peptide dual agonist comprises or consists of the sequence

Compound no. 37 SEQ ID NO: 31
H-Aib-DGTFISDY-K(γGlu-C16)-TAib-LDNLAARDFINWLIQTKITKNDWKHNITQ-OH.

In one embodiment the peptide dual agonist comprises or consists of the sequence

Compound no. 40 SEQ ID NO: 34
(C16-diacid)H-Aib-DGTFISDYST-Aib-LDNLAARDFINWLIQTKITKNDWKHNITQ-OH.

In one embodiment the peptide dual agonist comprises or consists of the sequence

Compound no. 41 SEQ ID NO: 35
(C16-diacid)H-Aib-EGTFISDYST-Aib-LDALAARDFINWLIQTKITKNDWKHNITQ-OH.

In one embodiment the peptide dual agonist comprises or consists of the sequence

Compound no. 42 SEQ ID NO: 36
(C16-diacid)H-Aib-DGTFISDYST-Aib-LDNLAAKDFINWLIQTKITKNDWKHNITQ-OH.

In one embodiment the peptide dual agonist comprises or consists of the sequence

Compound no. 44 SEQ ID NO: 4
(C10)H-Aib-DGTFISDYST-Aib-LDNLAARDFINWLIQTKITK-OH.

In one embodiment the peptide dual agonist comprises or consists of the sequence Compound no. 31 SEQ ID NO: 26 H-Aib-DGTFISDY-K(γGlu-C10)-TILDKLAARDFIEWLIATKITK-OH. This peptide dual agonist has both good potency and good physical stability.

### Activities of the Peptide Dual Agonists

In one embodiment a functional variant or fragment retains the same biological activity or capabilities as the native peptide or the peptide from which it is derived.

In one embodiment the peptide dual agonist including functional variants or fragments thereof is capable of one or more of:
a. binding to GIPR and GLP-2R, and/or
b. activation of GIPR and GLP-2R, and/or
c. stimulation of GIPR- and GLP-2R activation, such as GIPR- and GLP-2R-mediated CAMP production, and/or
d. inhibiting bone resorption, and/or
e. stimulating bone formation.

In one embodiment the peptide dual agonist including functional variants or fragments thereof is capable of
f. reducing recruitment of beta-arrestin 2 to the GIPR compared to native hGIP;
g. reducing GIPR internalization compared to native hGIP.

These effects may be desired because they may lead to a sustained pharmacological effect in long term treatment (weeks, months, years) and may not cause desensibilization of the GIPR. Desensibilization of the GIPR is discussed in Sameer et al. Mol Cell Biol. 2014 Oct. 1; 34 (19): 3618-29. doi: 10.1128/MCB.00256-14.

In one embodiment the peptide dual agonist as well as functional variants or fragments thereof is a full agonist of GIPR and GLP-2R.

In one embodiment the peptide dual agonists are capable of binding to and activating GIPR. In some embodiments, the GIPR is the human GIPR (Uniprot accession number P48546). In one embodiment the peptide dual agonists are capable of binding to and activating GLP-2R. In some embodiments, the GLP-2R is the human GLP-2R (Uniprot accession number 095838).

Activities of the peptide dual agonists of the present disclosure may be assessed experimentally by means of a CAMP assay, as the one described in “Examples” herein. Such a CAMP assay can be used to determine EC₅₀ and Eₘₐₓ, as well as other relevant parameters.

In one embodiment the peptide dual agonists provided herewith are capable of
a. activating the human GIPR with an efficacy (Eₘₐₓ values) which is at least 65% of the efficacy by which native human GIP activates the human GIPR, such as at least 70%, such as at least 75%, such as at least 80%, such as at least 85%, such as at least 90%, such as at least 95% of the efficacy by which native human GIP activates the human GIPR; and
b. activating the human GLP-2R with an efficacy (Eₘₐₓ values) which is at least 65% of the efficacy by which native human GLP-2 activates the human GLP-2R, such as at least 70%, such as at least 75%, such as at least 80%, such as at least 85%, such as at least 90%, such as at least 95% of the efficacy by which native human GLP-2 activates the human GLP-2R.

In one embodiment the peptide dual agonists provided herewith are capable of
activating the GIPR with the same or increased potency by which native GIP activates the GIPR; and
activating the GLP-2R with the same or increased potency by which native GLP-2 activates the GLP-2R.

In one embodiment the peptide dual agonists provided herewith are capable of activating the human GLP-1R with an efficacy (Eₘₐₓ values) which is at the most 80% of the efficacy by which native human GLP-1 activates the human GLP-1R, such as an efficacy (Eₘₐₓ values) which is at the most 70% of the efficacy by which native human GLP-1 activates the human GLP-1R, such as an efficacy (Eₘₐₓ values) which is at the most 60% of the efficacy by which native human GLP-1 activates the human GLP-1R.

In one embodiment the peptide dual agonists provided herewith
a. have an EC₅₀ towards the human GIPR of 10 nM or less; such as of 8 nM or less, such as of 7 nM or less, such as of 6 nM or less, such as of 5 nM or less, such as of 4 nM or less, such as of 3 nM or less, such as of 2 nM or less, such as of 1 nM or less, and
b. have an EC₅₀ towards the human GLP-2R of 50 nM, such as of 40 nM or less, such as of 30 nM or less, such as of 25 nM or less, such as of 20 nM or less, such as of 15 nM or less, such as of 10 nM or less.

In one embodiment the peptide dual agonists provided herewith have an EC₅₀ towards the human GLP-1R of 10 nM or more, such as of 12 nM or more, such as of 15 nM or more.

In one embodiment the peptide dual agonists provided herewith have an EC₅₀ towards the human GIPR that is at least 10 times lower than its EC₅₀ towards human GLP-1R, such as at least 12 times lower than its EC₅₀ towards human GLP-1R, such as at least 15 times lower than its EC₅₀ towards human GLP-1R.

In one embodiment the peptide dual agonists provided herewith have an EC₅₀ towards the human GLP-2R that is at least 10 times lower than its EC₅₀ towards human GLP-1R, such as at least 12 times lower than its EC₅₀ towards human GLP-1R, such as at least 15 times lower than its EC₅₀ towards human GLP-1R.

### Medicament/Medical Use

It is an aspect of the present disclosure to provide a peptide dual agonist, a nucleic acid construct encoding a peptide dual agonist, a delivery vehicle comprising a nucleic acid construct encoding a peptide dual agonist, as well a composition comprising the peptide dual agonist, as defined herein elsewhere, for use as a medicament.

It is thus an aspect of the present disclosure to provide a peptide dual agonist as defined herein for use in a method of inhibiting bone resorption and/or stimulating bone formation.

It is also an aspect of the present disclosure to provide a peptide dual agonist as defined herein for use in a method of inhibiting bone resorption and/or stimulating bone formation.

Also provided is the use of a peptide dual agonist as defined herein for the manufacture of a medicament for inhibiting bone resorption and/or stimulating bone formation.

Also provided is a method of inhibiting bone resorption and/or stimulating bone formation, said method comprising administering a therapeutically effective amount of a peptide dual agonist as defined herein to an individual in need thereof.

It is a further aspect of the present disclosure to provide a peptide dual agonist as defined herein for use in a method of treating a bone disorder. In one embodiment the bone disorder is a disorder associated with increased bone resorption and/or reduced bone formation. In one embodiment the bone disorder is associated with poor or reduced bone density.

Also provided is a peptide dual agonist as defined herein for use in a method of treating a bone disorder, including treating, preventing and alleviating said bone disorder.

Also disclosed is a peptide dual agonist disclosed herein for use in a method of treating a bone disorder.

Bone density or bone mineral density (BMD) is the amount of bone mineral in bone tissue. The concept is of mass of mineral per volume of bone (relating to density in the physics sense), although clinically it is measured by proxy according to optical density per square centimetre of bone surface upon imaging. Bone density measurement is used in clinical medicine as an indirect indicator of osteoporosis/osteopenia and fracture risk. It is measured by a procedure called densitometry. There is a statistical association between poor bone density and higher probability of fracture. Bone density measurements are used to screen people for osteoporosis risk and to identify those who might benefit from measures to improve bone strength.

The T-score is the relevant measure when screening for osteoporosis. It is the bone mineral density (BMD) at the site when compared to the young normal reference mean.

The criteria of the World Health Organization are:

Normal is a T-score of −1.0 or higher

Osteopenia is defined as between −1.0 and −2.5

Osteoporosis is defined as −2.5 or lower, meaning a bone density that is two and a half standard deviations below the mean of a young normal reference.

In one embodiment the bone disorder is associated with a T-score of −1.0 or lower, such as between −1.0 and −2.5, such as −2.5 or lower.

In one embodiment there is provided the use of a peptide dual agonist as defined herein for the manufacture of a medicament for treating a bone disorder.

Also provided is a method of treating a bone disorder, said method comprising administering a therapeutically effective amount of a peptide dual agonist as defined herein to an individual in need thereof.

An individual in need as referred to herein, is an individual that may benefit from the administration of a dual agonist peptide. Such an individual may suffer from a bone disorder or be in risk of suffering therefrom. The individual may be any human being, male or female, infant, middle-aged or old. The disorder to be treated or prevented in the individual may relate to the age of the individual, the general health of the individual, the medications used for treating the individual and whether or not the individual has a prior history of suffering from diseases or disorders that may have or have induced a bone density disorder.

In one embodiment the bone disorder is selected from the group consisting of osteopenia, osteoporosis, severe osteoporosis, post-menopausal osteoporosis, iodiopathic osteoporosis, osteomalacia, rickets, osteitis fibrosa cystica (OFC) and Paget's disease of bone.

In one embodiment the bone disorder is osteopenia.

In one embodiment the bone disorder is osteoporosis.

In one embodiment the bone disorder is post-menopausal osteoporosis.

In one embodiment the bone disorder is iodiopathic osteoporosis, such as juvenile iodopathic osteoporosis.

It is a further aspect of the present disclosure to provide a peptide dual agonist as defined herein for use in a method of treating osteoporosis, such as idiopathic osteoporosis, in an individual suffering from Duchenne muscular dystrophy (DMD) or cerebral Palsy.

In one embodiment the bone disorder is a secondary disorder, such as a secondary disorder in an individual suffering from Duchenne muscular dystrophy (DMD) or cerebral Palsy.

In one embodiment the bone disorder is a secondary disorder, such as a secondary disorder in an individual who is undergoing steroid, such as glucocorticoid treatment and/or is subject to immobility.

Idiopathic osteoporosis refers to the development of osteopenia and fractures with minimal or no trauma in otherwise young, healthy individuals who are not postmenopausal or have other, identifiable secondary causes of osteoporosis.

Also disclosed is a peptide dual agonist selected from the group consisting of SEQ ID NO: s 66 and 67, or a functional variant thereof, or a functional fragment thereof, for use in a method of treating a bone disorder selected from the group consisting of osteopenia, osteoporosis, severe osteoporosis, post-menopausal osteoporosis, idiopathic osteoporosis, osteomalacia, rickets, osteitis fibrosa cystica (OFC) and Paget's disease of bone.

In one embodiment there is provided a peptide dual agonist selected from the group consisting of SEQ ID NO:s 66 and 67, or a functional variant thereof, or a functional fragment thereof, for use in a method of treating osteopenia.

In one embodiment there is provided a peptide dual agonist selected from the group consisting of SEQ ID NO:s 66 and 67, or a functional variant thereof, or a functional fragment thereof, for use in a method of treating osteoporosis.

In one embodiment there is provided a peptide dual agonist selected from the group consisting of SEQ ID NO:s 66 and 67, or a functional variant thereof, or a functional fragment thereof, for use in a method of treating osteoporosis in an individual suffering from Duchenne muscular dystrophy (DMD) or cerebral Palsy.

### Combination Therapy

It is also an aspect to provide a peptide dual agonist as defined herein for use in combination with a further active pharmaceutical ingredient. Said further active ingredient is in one embodiment useful for treating a bone disorder, such as a bone disorder associated with reduced bone density.

In one embodiment the further active pharmaceutical ingredient is selected from the group consisting of Bisphosphonates including Alendronate (Fosamax), Risedronate (Actonel, Atelvia, Benet), Ibandronate (Boniva), Zoledronic acid (Reclast, Aclasta, Zometa), Etidronic acid (Didronel), Pamidronic acid (Aredia/Pamimed), Tiludronic acid (Skelid); estrogen replacement therapy; hormone therapies; hormone-like medications including raloxifene (Evista); Calcitonin (Fortical and Miacalcin), Denosumab (Prolia); Teriparatide (Forteo); Vitamin D (alfacalcidol or calcitriol); calcium or phosphorus supplement.

In one embodiment the further active pharmaceutical ingredient increases the half-life of the present peptide dual agonist. In one embodiment the further active pharmaceutical ingredient is an inhibitor of dipeptidyl peptidase 4 (DPP-4/DDP-IV inhibitors), or gliptins. Examples include Sitagliptin, Vildagliptin, Saxagliptin, Linagliptin, Gemigliptin, Anagliptin, Teneligliptin, Alogliptin, Trelagliptin, Dutogliptin, and Omarigliptin (MK-3102).

### Nucleic Acid Construct Encoding Dual Agonist Peptide

In one embodiment there is provided a nucleic acid construct encoding a dual agonist peptide as defined herein. In one embodiment said nucleic acid construct will be able to continuously express said peptide for a prolonged period of time; such as at least 1 month, for example at least 2 months, such as at least 3 months, for example at least 4 months, such as at least 5 months, for example at least 6 months, such as at least 7 months, for example at least 8 months, such as at least 9 months, for example at least 12 months.

It is thus an aspect to provide a nucleic acid construct encoding a peptide dual agonist as defined herewith.

In one embodiment there is provided a nucleic acid construct encoding a peptide dual agonist of SEQ ID NO: 66 or 67, or a functional variant thereof, or a functional fragment thereof, or any peptide dual agonist disclosed herein.

It is also an aspect to provide a nucleic acid construct encoding a dual agonist peptide as defined herein for use in a method of treating a bone disorder.

By nucleic acid construct is understood a genetically engineered nucleic acid. The nucleic acid construct may be a non-replicating and linear nucleic acid, a circular expression vector or an autonomously replicating plasmid. A nucleic acid construct may comprise several elements such as, but not limited to genes or fragments of same, promoters, enhancers, terminators, poly-A tails, linkers, polylinkers, operative linkers, multiple cloning sites (MCS), markers, STOP codons, internal ribosomal entry sites (IRES) and host homologous sequences for integration or other defined elements. It is to be understood that the nucleic acid construct according to the present invention may comprise all or a subset of any combination of the above-mentioned elements. Methods for engineering nucleic acid constructs are well known in the art (see, e.g., Molecular Cloning: A Laboratory Manual, Sambrook et al., eds., Cold Spring Harbor Laboratory, 2nd Edition, Cold Spring Harbor, N.Y., 1989). Further, nucleic acid constructs according to the present invention may be synthesized without template, and may be obtained from various commercial suppliers (e.g. Genscript Corporation).

In one embodiment, the nucleic acid constructs are naked DNA constructs comprising sequences encoding the dual agonist peptide.

### Delivery Vehicles

It is also an aspect to provide the nucleic acid construct as described herein above comprised within a delivery vehicle. A delivery vehicle is an entity whereby a nucleotide sequence or polypeptide or both can be transported from at least one media to another. Delivery vehicles are generally used for expression of the sequences encoded within the nucleic acid construct and/or for the intracellular delivery of the construct or the polypeptide encoded therein.

In one embodiment, there is provided a delivery vehicle comprising the nucleic acid construct as defined herein. A delivery vehicle may be selected from the group consisting of: RNA based vehicles, DNA based vehicles/vectors, lipid based vehicles (such as a liposome), polymer based vehicles (such as a cationic polymer DNA carrier), colloidal gold particles (coating) and virally derived DNA or RNA vehicles or vectors.

Methods of non-viral delivery include physical (carrier-free delivery) and chemical approaches (synthetic vector-based delivery).

Physical approaches, including needle injection, gene gun, jet injection, electroporation, ultrasound, and hydrodynamic delivery, employ a physical force that permeates the cell membrane and facilitates intracellular gene transfer. Said physical force may be electrical or mechanical.

Examples of chemical delivery vehicles include, but are not limited to: biodegradable polymer microspheres, lipid based formulations such as liposome carriers, cationically charged molecules such as liposomes, calcium salts or dendrimers, lipopolysaccharides, polypeptides and polysaccharides.

Another embodiment comprises a vector which herein is denoted a viral vector (i.e. not a virus) as a delivery vehicle. Viral vectors according to the present invention are made from a modified viral genome, i.e. the actual DNA or RNA forming the viral genome, and introduced in naked form. Thus, any coat structures surrounding the viral genome made from viral or non-viral proteins are not part of the viral vector.

The virus from which the viral vector is derived may be selected from the non-exhaustive group of: adenoviruses, retroviruses, lentiviruses, adeno-associated viruses, herpesviruses, vaccinia viruses, foamy viruses, cytomegaloviruses, Semliki forest virus, poxviruses, RNA virus vector and DNA virus vector. Such viral vectors are well known in the art.

In one embodiment, said viral vectors may be selected from the group consisting of adenoviruses, lentiviruses, adeno-associated viruses (AAV) and recombinant adeno-associated viruses (rAAV). In one preferred embodiment, said viral vector is a therapeutic rAAV vector such as a therapeutic rAAV vector.

An adenovirus is a group of double-stranded DNA containing viruses. Adenoviruses can be genetically modified making them replication incompetent or conditionally replication incompetent. In this form, as adenoviral constructs or adenovectors, they can be used as gene delivery vehicles for vaccination or gene therapy.

### Recombinant Cell

Another aspect of relates to a cell comprising the nucleic acid construct as defined herein. Such a recombinant cell can be used a tool for in vitro research, as a delivery vehicle for the nucleic acid construct or as part of a gene-therapy regime. The nucleic acid construct can be introduced into cells by techniques well known in the art which include microinjection of DNA into the nucleus of a cell, transfection, electroporation, lipofection/liposome fusion and particle bombardment. Suitable cells include autologous and non-autologous cells, and may include xenogenic cells.

### Method of Preparation (Peptide)

The dual agonist peptides as defined herein may be prepared by any methods known in the art; such as by standard peptide-preparation techniques including solution synthesis or Merrifield-type solid phase synthesis.

In one embodiment a peptide according to the invention is synthetically made or produced. The methods for synthetic production of peptides are well known in the art. Detailed descriptions as well as practical advice for producing synthetic peptides may be found in Synthetic Peptides: A User's Guide (Advances in Molecular Biology), Grant G. A. ed., Oxford University Press, 2002, or in: Pharmaceutical Formulation: Development of Peptides and Proteins, Frokjaer and Hovgaard eds., Taylor and Francis, 1999.

In one embodiment the peptide or peptide sequences of the invention are produced synthetically, in particular, by the Sequence Assisted Peptide Synthesis (SAPS) method, by solution synthesis, by Solid-phase peptide synthesis (SPPS) such as Merrifield-type solid phase synthesis, by recombinant techniques (production by host cells comprising a first nucleic acid sequence encoding the peptide operably associated with a second nucleic acid capable of directing expression in said host cells) or enzymatic synthesis. These are well-known to the skilled person.

Peptides may be synthesised either batch-wise on a fully automated peptide synthesiser using 9-fluorenylmethyloxycarbonyl (Fmoc) or tert-Butyloxycarbonyl (Boc) as N-a-amino protecting group and suitable common protection groups for side-chain functionalities.

After purification such as by reversed phase HPLC, peptides may be further processed to obtain for example C- or N-terminal modified isoforms. The methods for terminal modification are well-known in the art.

For example, the following methods for conjugation of fatty acid can be used. After the last N-terminal N-Fmoc is removed, the N-terminal amino group is acylated by the treatment of 3 equiv 1,16-Hexadecanedioic acid in the presence of 3 equiv HATU and 6 equiv DIPEA. As an alternative, Lysine with Dde protected side chain is used for the solid phase peptide synthesis in the acylation position. After the peptide chain is assembled, the N-terminal amino group is capped by 3 equiv Boc2O in the presence of 6 equiv DIPEA. Then the protection group Dde is removed by the treatment of 2% hydrazine/DMF (v/v). As an alternative, 3 equiv palmitoyl chloride can be used for palmitoylation in the presence of 6 equiv DIPEA. Or 3 equiv 1, 16-Hexadecanedioic acid can be activated by HATU/DIPEA and then conjugated to the side chain of Lysine. As a further alternative, Fmoc-γ-Glu(tBu)-OH or Fmoc-4-Abu-OH or Fmoc-β-Ala-OH or Fmoc-AEEAc-OH are conjugated to the side chain of Lys using a standard protocol of DIC/HOBT. The N-terminal amino groups of unnatural amino acids is then palmitoylated by the treatment of 3 equiv palmitoyl chloride in the presence of 6 equiv DIPEA.

### Pharmaceutical Composition and Formulation

Whilst it is possible for the dual agonist peptide as defined herewith to be administered as the raw chemical (peptide), it is sometimes preferred to present them in the form of a pharmaceutical formulation. Such a pharmaceutical formulation may be referred to as a pharmaceutical composition, pharmaceutically acceptable composition or pharmaceutically safe composition.

Accordingly, also provided is a pharmaceutical formulation, which comprises a dual agonist peptide as defined herein, or a pharmaceutically acceptable salt or ester thereof, and a pharmaceutically acceptable carrier, excipient and/or diluent. The pharmaceutical formulations may be prepared by conventional techniques, e.g. as described in Remington: The Science and Practice of Pharmacy 2005, Lippincott, Williams & Wilkins.

Pharmaceutically acceptable salts of the instant peptide compounds, where they can be prepared, are also intended to be covered. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The peptide compounds may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

The pharmaceutically acceptable salt of the peptide of the invention is preferably in solution with a physiologically acceptable pH, i.e. the solution comprising the peptide salt preferably has a pH acceptable for clinical use.

Provided herewith is a composition comprising a peptide dual agonist as defined herein; or a multimeric compound comprising said peptide dual agonist; or a nucleic acid construct comprising/encoding said peptide dual agonist.

### Administration and Dosage

In one embodiment of the present disclosure, a peptide or a nucleic acid construct encoding said peptide, or a composition comprising a peptide as defined herein is administered to individuals in need of treatment in pharmaceutically effective doses or a therapeutically effective amount. The dosage requirements will vary with the particular drug composition employed, the route of administration and the particular subject being treated, which depend on the severity and the sort of the disorder as well as on the weight and general state of the subject. It will also be recognized by one skilled in the art that the optimal quantity and spacing of individual dosages will be determined by the nature and extent of the condition being treated, the form, route and site of administration, and the particular patient being treated, and that such optima can be determined by conventional techniques. It will also be appreciated by one of skill in the art that the optimal course of treatment, i.e., the number of doses of a compound given per day for a defined number of days, can be ascertained using conventional course of treatment determination tests.

In one embodiment the peptide or composition is administered at least once daily, such as once daily, such as twice daily, such as thrice daily, such as four times daily, such as five times daily.

A dose may also be administered in intermittent intervals, or intervals, whereby a dose is not administered every day. Rather one or more doses may be administered every second day, every third day, every fourth day, every fifth day, every sixth day, every week, every second week, every third week, every fourth week, every fifth week, every sixth week, or intervals within those ranges (such as every 2 to 4 weeks, or 4 to 6 weeks).

In one embodiment the peptide or composition is administered once daily.

In one embodiment the peptide or composition is administered once daily, such as overnight, when bone resorption is highest.

In one embodiment the peptide or composition is administered once weekly.

The skilled person knows that if the number of daily administrations is increased, the dose to be administered in each administration may be decreased accordingly. Likewise, if the duration of each administration is decreased, the dosage may be increased accordingly.

### Routes of Administration

It will be appreciated that the preferred route of administration will depend on the general condition and age of the subject to be treated, the nature of the condition to be treated, the location of the tissue to be treated in the body and the active ingredient chosen.

### Systemic Treatment

For systemic treatment the route of administration is capable of introducing the peptide, nucleic acid construct encoding said peptide, or the composition comprising the peptide into the blood stream to ultimately target the sites of desired action.

Such routes of administration are any suitable routes, such as an enteral route (including the oral, rectal, nasal, pulmonary, buccal, sublingual, transdermal, intracisternal and intraperitoneal administration), and/or a parenteral route (including subcutaneous, intramuscular, intrathecal, intracerebral, intravenous and intradermal administration).

### Parenteral Administration

Parenteral administration is any administration route not being the oral/enteral route whereby the medicament avoids first-pass degradation in the liver. Accordingly, parenteral administration includes any injections and infusions, for example bolus injection or continuous infusion, such as intravenous administration, intramuscular administration or subcutaneous administration. Furthermore, parenteral administration includes inhalations and topical administration.

Accordingly, compound be administered topically to cross any mucosal membrane of an animal to which the biologically active substance is to be given, e.g. in the nose, vagina, eye, mouth, genital tract, lungs, gastrointestinal tract, or rectum, preferably the mucosa of the nose, or mouth, and accordingly, parenteral administration may also include buccal, sublingual, nasal, rectal, vaginal and intraperitoneal administration as well as pulmonal and bronchial administration by inhalation or installation. Also, the agent may be administered topically to cross the skin.

### Local Treatment

The peptide, or a nucleic acid construct encoding said peptide, or a composition comprising a peptide as defined herein may in one embodiment be used as a local treatment, i.e. be introduced directly to the site(s) of action. Accordingly, it may be applied to the skin or mucosa directly, or it may be injected into the site of action, for example into the diseased tissue or to an end artery leading directly to the diseased tissue.

### Kit-of-Parts

The present invention also relates to a kit-of-parts comprising one or more of the agents described above (a peptide, a nucleic acid construct or a composition), and at least one additional or further component.

A kit of parts in one embodiment comprises one or more of the agents as defined herein for treatment, prevention or alleviation of a bone disorder osteoporosis and osteopenia. Kits as defined herein allows for simultaneous, sequential or separate administration of the active agent according to the present invention and/or one or more second active ingredients as described herein elsewhere.

### EXAMPLES

### Example 1. Peptide Dual Agonists of GIPR and GLP-2R

### Materials and Methods

### Materials

All applied peptides were synthesized by WuXi AppTec, Shanghai, China, with a purity of at least 95% or above, determined by HPLC analysis, and the correct molecular weight was checked using mass spectrometry. The human GIPR, GLP-2R and GLP-1R applied in the in vitro experiments can be purchased by Origene, Rockville, USA. Human N-terminally SNAP-tagged GIPR was custom produced by Cisbio, Codolet, France. C10, C12 or C16 or C16-diacid acyl groups were added on position 1, 5, 7, 11, 12, 15 as provided in Table 1 to improve the selectivity of the dual agonists.

### Results

The peptide dual agonists listed in Table 1 were obtained

TABLE 1
Peptides dual agonists of GIPR and GLP-2R.

| Compound | SEQ | | N- | C- | Lip. | | |
|---|---|---|---|---|---|---|---|
| no. | ID NO. | Length | term | term | Pos. | Fatty acid | Linker |
| 1 | 1 | 33 | | | N-term | C16 | |
| 2 | 2 | 32 | | | N-term | C16 | |
| 3 | 3 | 43 | | | N-term | C10 | |
| 4 | 1 | 33 | | | N-term | C16 | |
| 5 | 4 | 33 | | | N-term | C16 | |
| 6 | 5 | 33 | | | N-term | C16 | |
| 7 | 6 | 33 | | | N-term | C16 | |
| 8 | 7 | 32 | | | N-term | C16 | |
| 9 | 8 | 33 | | | 5 | C16 | yGlu |
| 10 | 9 | 33 | | | 7 | C16 | yGlu |
| 11 | 10 | 33 | | | 11 | C16 | yGlu |
| 12 | 11 | 33 | | | 12 | C16 | yGlu |
| 13 | 12 | 33 | | | 15 | C16 | yGlu |
| 14 | 13 | 42 | | NH2 | 11 | C16 | yGlu |
| 15 | 14 | 42 | | COOH | N-term | C16 | |
| 16 | 15 | 42 | | COOH | N-term | C16 | |
| 17 | 4 | 33 | | OH | N-term | C16-diacid | |
| 18 | 4 | 33 | | OH | N-term | C10-diacid | |
| 19 | 16 | 33 | | | N-term | C16-diacid | |
| 20 | 17 | 33 | | | N-term | C16-diacid | |
| 21 | 18 | 33 | | | N-term | C16-diacid | |
| 22 | 19 | 33 | | | N-term | C16-diacid | |
| 23 | 20 | 33 | | | N-term | C16-diacid | |
| 24 | 21 | 33 | | | 5 | C10 | yGlu |
| 25 | 21 | 33 | | | 5 | C16-diacid | yGlu |
| 26 | 8 | 33 | | | 5 | C10 | yGlu-yGlu |
| 27 | 22 | 33 | | | 5 | C16 | yGlu |
| 28 | 21 | 33 | | | 5 | C16 | yGlu-yGlu |
| 29 | 23 | 33 | | | 11 | C16 | yGlu |
| 30 | 25 | 33 | | | 11 | C10 | yGlu |
| 31 | 26 | 33 | | | 11 | C10 | yGlu |
| 32 | 27 | 33 | | | 11 | C16 | yGlu |
| 33 | 28 | 33 | | | 11 | C16 | yGlu |
| 34 | 24 | 33 | Ac | | 11 | C16 | yGlu |
| 35 | 29 | 33 | | | 12 | C16 | yGlu |
| 36 | 30 | 33 | Ac | | 12 | C16 | yGlu |
| 37 | 31 | 42 | | | 11 | C16 | yGlu |
| 38 | 32 | 42 | Ac | | 11 | C16 | yGlu |
| 39 | 33 | 42 | | | N-term | C16-diacid | |
| 40 | 34 | 42 | | | N-term | C16-diacid | |
| 41 | 35 | 42 | | | N-term | C16-diacid | |
| 42 | 36 | 42 | | | N-term | C16-diacid | |
| 43 | 37 | 42 | | | N-term | C16-diacid | |
| 44 | 4 | 33 | | | N-term | C10 | |
| 45 | 38 | 33 | | | N-term | C16 | |
| 46 | 39 | 33 | | | N-term | C16 | |
| 48 | 41 | 42 | | | N-term | C16 | |
| 49 | 42 | 42 | | | N-term | C16 | |
| 50 | 43 | 42 | | | N-term | C16 | |
| 51 | 44 | 42 | | | N-term | C16 | |
| 52 | 45 | 42 | | | N-term | C16 | |
| 53 | 33 | 42 | | | N-term | C16 | |
| 54 | 46 | 42 | | | 11 | C10 | yGlu |
| 56 | 47 | | | | | | |
| 57 | 48 | 33 | | | 30 | C16 | |
| 58 | 48 | 33 | | | C-term | C16 | |
| 60 | 49 | 33 | | | C-term | C18-diacid | 2xAEEA-yGlu |
| 61 | 50 | 33 | | | C-term | C18-diacid | 2xAEEA-yGlu |
| 62 | 51 | 33 | | | 16 | C18-diacid | 2xAEEA-yGlu |
| 63 | 52 | 33 | | | C-term | C10 | |
| 64 | 53 | 33 | | | 16 | C18-diacid | 2xAEEA-yGlu |
| 65 | 54 | 33 | | | C-term | C10 | |
| 66 | 55 | 33 | | | C-term | C14 | |

### Example 2

### Methods

COS-7 cells were cultured at 10% CO₂ and 37° C. in Dulbecco's Modified Eagle Medium (DMEM) 1885 supplemented with 10% fetal bovine serum (FBS), 2 mM glutamine, 180 units/ml penicillin, and 45 g/ml streptomycin. Transient transfection of COS-7 cells was performed using the calcium phosphate precipitation method as previously described (van der Velden et al., 2021) prior to experiment.

Transiently transfected COS-7 cells expressing either human GIPR, GLP-2R or GLP-1R were seeded in white 96-well plates the day after transfection with a density of 35.000 cells/well. The next day the assay was initiated by washing with HEPES buffered saline (HBS) and followed by an incubation step with assay buffer (HBS added 1 mM isobutyl-1-methylxanthine (IBMX)) for 30 min at 37° C. The ligands were then added and incubated for an additional 30 min at 37° C. After ligand incubation, the HitHunter® CAMP assay (Eurofins DiscoverX, Fremont, 150 USA) was carried out according to the manufacturer's instructions. Luminescence was measured by PerkinElmer™ EnVision 2014 Multilabel Reader (PerkinElmer, Waltham, MA). The potencies (EC₅₀ values) were determined by nonlinear regression using GraphPad Prism 9 (San Diego, California, USA). Sigmoid curves were fitted logistically. The efficacies (Eₘₐₓ values) were determined by normalization to the highest concentration applied of the endogenous ligand for each receptor.

### Results

It can be seen from table 2 and 3 that attaching a fatty acid molecule at an amino acid in the N-terminal region (residues 1 to 15 of a peptide according to the present disclosure), such as e.g. in position 5, 7, 8, 11, 12, 15 or the N-terminus lead to potent dual GIPR/GLP-2R agonists only or predominantly targeting the GIPR and GLP-2R and not the GLP-1R, by decreasing potency and/or efficacy at the GLP-1R. See for comparison the compounds in Table 4, in particular Compound no.: 63, 65, 64, 62, 60 and 61, which have a fatty acid attached at the C-terminal amino acid residue or at residue at position 16, and which are capable of activating hGLP-1R in addition to hGIP and hGLP-2R.

Aib at position 2 and/or 13 can increase potency at GIPR and/or GLP-2R. Aib at position 2 and/or 13 can also induce more selectivity towards the GLP-1R.

Ala at position 2 seems to result in dual agonist peptides with imbalanced potency and/or efficacy towards GIPR rather than GLP-2R.

Fatty acids of increasing length (e.g. C16 vs C10) may induce more selectivity, possibly due to sterical hindrance which in the specified positions are more tolerated at the GIPR and GLP-2R than the GLP-1R.

A yGlu (gamma-glutamate) linker may also induce more selectivity, possibly due to sterical hindrance which in the specified positions are more tolerated at the GIPR and GLP-2R than the GLP-1R.

Isoleucine at position 7 in combination with attaching a fatty acid molecule at an amino acid in the N-terminal region, such as e.g. in position 5, 7, 8, 11, 12, 15 or the N-terminus, decreases potency and/or efficacy at the GLP-1R.

TABLE 2
EC₅₀ (potency) and Emax (efficacy) of the tested peptide dual agonists
of GIPR and GLP-2R with respect to human GIPR and human GLP-2R.

| | GIPR | | | | | | GLP-2R | | | | | |
|---|---|---|---|---|---|---|---|---|---|---|---|---|
| | | | EC₅₀ | | | | | | EC₅₀ | | | |
| Compound | PEC₅₀ | | (nM) | Efficacy | | | logEC₅₀ | | (nM) | Efficacy | | |
| no. | GIPR | SD | GIPR | GIPR | SD | n= | GLP-2R | SD | GLP-2R | GLP-2R | SD | n= |
| 1 | 9.2 | 0.2 | 0.584 | 96 | 17 | 3 | 7.6 | 0.2 | 27.1 | 81 | 14 | 3 |
| 2 | 9.3 | 0.2 | 0.464 | 80 | 17 | 3 | 7.3 | NA | 50.1 | 59 | 22 | 3 |
| 3 | 9.2 | 0.2 | 0.584 | 76 | 28 | 3 | 8.3 | 0.2 | 5.0 | 58 | 13 | 3 |
| 4 | 8.4 | 0.2 | 3.687 | 96 | 36 | 3 | 7.6 | 0.3 | 27.1 | 86 | 18 | 3 |
| 5 | 9.5 | 0.2 | 0.355 | 111 | 6 | 2 | 9.0 | 0.1 | 1.1 | 109 | 20 | 2 |
| 6 | 9.1 | 0.1 | 0.794 | 108 | 5 | 2 | 8.5 | 0.0 | 3.2 | 91 | 13 | 2 |
| 7 | 9.1 | 0.1 | 0.891 | 114 | 1 | 2 | 8.4 | 0.1 | 4.0 | 101 | 14 | 2 |
| 8 | 8.8 | 0.0 | 1.585 | 108 | 5 | 2 | 8.3 | 0.4 | 5.0 | 106 | 23 | 2 |
| 9 | 9.9 | 0.1 | 0.141 | 110 | 8 | 2 | 8.6 | 0.0 | 2.5 | 91 | 4 | 2 |
| 10 | 8.9 | 0.1 | 1.259 | 102 | 8 | 2 | 7.5 | 0.3 | 31.6 | 23 | 6 | 2 |
| 11 | 9.2 | 0.1 | 0.681 | 115 | 8 | 3 | 8.7 | 0.2 | 2.2 | 87 | 6 | 3 |
| 12 | 9.6 | 0.0 | 0.251 | 107 | 10 | 2 | 9.1 | 0.1 | 0.9 | 79 | 6 | 2 |
| 13 | 9.1 | 0.1 | 0.794 | 95 | 20 | 2 | 8.2 | 0.1 | 6.3 | 95 | 2 | 2 |
| 14 | 9.6 | 0.2 | 0.266 | 101 | 15 | 5 | 8.5 | 0.4 | 3.5 | 114 | 15 | 5 |
| 15 | 8.5 | 0.3 | 2.929 | 106 | 19 | 3 | 7.3 | 0.1 | 50.1 | 70 | 22 | 3 |
| 16 | 9.4 | 0.2 | 0.447 | 99 | 16 | 2 | 7.8 | 0.0 | 15.8 | 64 | 20 | 2 |
| 17 | 9.2 | 0.2 | 0.681 | 98 | 5 | 3 | 8.8 | 0.2 | 1.7 | 90 | 17 | 3 |
| 18 | 8.4 | 0.5 | 4.217 | 77 | 10 | 4 | 9.3 | 0.3 | 0.6 | 90 | 8 | 4 |
| 19 | 9.2 | 0.2 | 0.708 | 98 | 0 | 2 | 8.6 | 0.2 | 2.8 | 81 | 21 | 2 |
| 20 | 9.1 | 0.4 | 0.858 | 93 | 9 | 3 | 8.9 | 0.1 | 1.3 | 88 | 3 | 3 |
| 21* | 9.2 | 0.1 | 0.631 | 86 | 1 | 2 | 8.1 | 0.3 | 7.9 | 58 | 4 | 2 |
| 22 | 9.0 | 0.3 | 1.096 | 91 | 1 | 5 | 8.6 | 0.5 | 2.5 | 95 | 16 | 5 |
| 23 | 8.8 | 0.4 | 1.778 | 107 | 0 | 2 | 8.8 | 0.4 | 1.8 | 86 | 1 | 2 |
| 24 | 9.8 | 0.1 | 0.158 | 113 | 6 | 4 | 8.5 | 0.2 | 3.5 | 90 | 15 | 4 |
| 25 | 10.0 | 0.2 | 0.106 | 93 | 16 | 4 | 9.0 | 0.2 | 1.1 | 88 | 14 | 4 |
| 26 | 8.5 | 0.3 | 3.162 | 80 | 4 | 2 | 8.2 | 0.0 | 6.3 | 91 | 16 | 2 |
| 27 | 8.8 | 0.2 | 1.778 | 98 | 1 | 2 | 8.1 | 0.3 | 7.9 | 108 | 27 | 2 |
| 28 | 8.8 | 0.3 | 1.585 | 105 | 2 | 2 | 8.8 | 0.1 | 1.6 | 99 | 1 | 2 |
| 29 | 9.0 | 0.3 | 0.926 | 104 | 13 | 3 | 8.6 | 0.3 | 2.3 | 124 | 23 | 3 |
| 30 | 8.7 | 0.3 | 1.995 | 85 | 16 | 2 | 8.6 | 0.1 | 2.8 | 81 | 4 | 2 |
| 31 | 9.4 | 0.2 | 0.398 | 89 | 14 | 3 | 8.4 | 0.2 | 3.7 | 90 | 27 | 3 |
| 32 | 9.5 | 0.4 | 0.341 | 89 | 7 | 2 | 8.0 | 0.0 | 10.0 | 84 | 18 | 2 |
| 33 | 9.5 | 0.3 | 0.355 | 101 | 9 | 5 | 8.9 | 0.3 | 1.4 | 97 | 20 | 4 |
| 34 | 8.0 | 0.0 | 10.00 | 75 | 10 | 2 | 7.9 | 0.1 | 14.1 | 103 | 1 | 2 |
| 35 | 9.9 | 0.3 | 0.141 | 106 | 11 | 4 | 9.1 | 0.2 | 0.9 | 88 | 10 | 4 |
| 36 | 9.0 | 0.1 | 1.000 | 92 | 6 | 2 | 8.4 | 0.2 | 4.5 | 76 | 11 | 2 |
| 37 | 9.5 | 0.4 | 0.302 | 95 | 11 | 5 | 9.1 | 0.4 | 0.7 | 97 | 7 | 4 |
| 38 | 8.5 | 0.4 | 3.548 | 81 | 11 | 2 | 8.5 | 0.2 | 3.5 | 89 | 1 | 2 |
| 39 | 8.8 | 0.1 | 1.778 | 99 | 4 | 2 | 9.0 | 0.1 | 1.0 | 78 | 11 | 2 |
| 40 | 9.0 | 0.3 | 1.000 | 94 | 4 | 3 | 9.0 | 0.1 | 1.1 | 96 | 14 | 3 |
| 41 | 9.2 | 0.1 | 0.708 | 91 | 7 | 2 | 8.8 | 0.2 | 1.8 | 74 | 18 | 2 |
| 42 | 9.1 | 0.1 | 0.891 | 91 | 2 | 2 | 8.6 | 0.1 | 2.8 | 93 | 8 | 2 |
| 43 | 8.7 | 0.1 | 1.995 | 101 | 16 | 2 | 8.6 | 0.1 | 2.8 | 73 | 21 | 2 |
| 44 | 9.0 | 0.5 | 1.068 | 110 | 16 | 7 | 8.5 | 0.4 | 2.9 | 100 | 7 | 7 |
| 45 | 8.6 | 0.2 | 2.712 | 91 | 13 | 3 | 7.3 | 0.4 | 29.3 | 59 | 32 | 3 |
| 46 | 8.8 | 0.4 | 1.585 | 98 | 3 | 3 | 7.6 | 0.3 | 23.3 | 82 | 35 | 3 |
| 47 | N/A | | | N/A | | | N/A | | | N/A | | |
| 48 | 8.6 | 0.1 | 2.818 | 112 | 28 | 2 | 7.6 | 0.4 | 25.1 | 69 | 21 | 2 |
| 49 | 8.6 | 0.1 | 2.818 | 105 | 9 | 2 | 7.5 | 0.1 | 35.5 | 92 | 1 | 2 |
| 50 | 9.1 | 0.4 | 0.891 | 101 | 32 | 2 | 8.0 | 0.0 | 10.0 | 70 | 15 | 2 |
| 51 | 9.4 | 0.0 | 0.398 | 104 | 1 | 2 | 7.9 | 0.0 | 12.6 | 66 | 19 | 2 |
| 52 | 9.6 | 0.4 | 0.251 | 96 | 5 | 2 | 8.1 | 0.2 | 7.9 | 68 | 26 | 3 |
| 53 | 9.5 | 0.3 | 0.316 | 90 | 2 | 2 | 8.2 | 0.4 | 4.0 | 79 | 9 | 2 |
| 54 | 8.9 | 0.2 | 1.166 | 86 | 12 | 3 | 8.9 | 0.4 | 1.3 | 87 | 24 | 3 |
| 55 | 7.8 | 0.3 | 15.849 | 103 | 24 | 2 | 7.4 | 0.3 | 36.9 | 50 | 34 | 3 |
| 57 | 7.9 | 0.1 | 12.589 | 97 | 13 | 2 | no | | | no | | |
| | | | | | | | activation | | | activation | | |
| 58 | 8.9 | 0.9 | 1.413 | 74 | 5 | 2 | no | | | no | | |
| | | | | | | | activation | | | activation | | |

*not fully dissolved

TABLE 3
EC₅₀ (potency) and Emax (efficacy) of the tested peptide dual
agonists of GIPR and GLP-2R with respect to human GLP-1R.

| Compound | logEC₅₀ | | EC₅₀ (nM) | Efficacy | | |
|---|---|---|---|---|---|---|
| no. | GLP-1R | SD | GLP-1R | GLP-1R | SD | n= |
| 1 | no activation | | | | | 3 |
| 2 | no activation | | | | | 3 |
| 3 | no activation | | | | | 3 |
| 4 | no activation | | | | | 3 |
| 5 | 7.8 | 0.1 | 15.85 | 6 | 2 | 2 |
| 6 | 7.4 | 0.1 | 39.81 | 34 | 3 | 2 |
| 7 | 7.4 | 0.6 | 39.81 | 13 | 1 | 2 |
| 8 | 8.0 | 1.4 | 10.00 | 19 | 8 | 2 |
| 9 | 7.2 | 0.1 | 70.79 | 32 | 1 | 2 |
| 10 | 7.0 | — | 100.00 | 10 | 14 | 2 |
| 11 | 7.1 | 0.4 | 89.13 | 11 | 1 | 2 |
| 12 | 7.7 | 0.1 | 19.95 | 62 | 15 | 2 |
| 13 | no activation | | | 4 | 5 | 2 |
| 14 | no activation | | | 0 | 0 | 5 |
| 15 | no activation | | | 0 | 0 | 3 |
| 16 | 7.9 | — | 12.59 | 2 | 2 | 2 |
| 17 | no activation | | | 5 | 9 | 3 |
| 18 | no activation | | | 10 | 14 | 2 |
| 19 | no activation | | | | | 2 |
| 20 | no activation | | | | | 3 |
| 21* | 7.3 | — | 50.12 | 10 | 14 | 2 |
| 22 | no activation | | | | | 3 |
| 23 | no activation | | | | | 2 |
| 24 | no activation | | | | | 3 |
| 25 | no activation | | | | | 3 |
| 26 | no activation | | | 14 | 20 | 2 |
| 27 | no activation | | | 0 | 0 | 2 |
| 28 | 7.2 | — | 63.10 | 14 | 20 | 2 |
| 29 | no activation | | | 0 | 0 | 3 |
| 30 | no activation | | | 10 | 14 | 2 |
| 31 | 7.4 | — | 39.81 | 6 | 10 | 3 |
| 32 | no activation | | | | | 2 |
| 33 | no activation | | | | | 2 |
| 34 | no activation | | | | | 2 |
| 35 | 10.0 | 1.1 | 0.11 | 31 | 14 | 3 |
| 36 | 11.0 | — | 0.01 | 19 | NA | 2 |
| 37 | no activation | | | | | 4 |
| 38 | no activation | | | | | 2 |
| 39 | no activation | | | | | 2 |
| 40 | no activation | | | | | 3 |
| 41 | 7.3 | — | 50.12 | 14 | 19 | 2 |
| 42 | no activation | | | | | 2 |
| 43 | no activation | | | | | 2 |
| 44 | 7.7 | 0.3 | 18.48 | 19 | 18 | 6 |
| 45 | no activation | | | | | |
| 46 | no activation | | | | | |
| 47 | no activation | | | | | |
| 48 | no activation | | | | | |
| 49 | no activation | | | | | |
| 50 | no activation | | | | | |
| 51 | no activation | | | | | |
| 52 | no activation | | | | | |
| 53 | no activation | | | | | |
| 54 | 7.4 | N/A | 39.81 | 8 | 11 | 3 |
| 55 | 7.3 | 0.2 | 46.42 | 77 | 5 | 3 |
| 57 | 7.0 | 0.1 | 100.00 | 73 | 6 | 3 |
| 58 | 7.1 | 0.6 | 85.77 | 69 | 8 | 3 |

*Not fully dissolved

TABLE 4A
EC₅₀ (potency) and Emax (efficacy) of the comparative
peptide with respect to human GIPR.

| Compound | pEC₅₀ | | EC₅₀ (nM) | Efficacy | | |
|---|---|---|---|---|---|---|
| no. | GIPR | SD | GIPR | GIPR | SD | n= |
| 66 | 8.8 | 0.2 | 1.468 | 97 | 6 | 3 |
| 63 | 9.6 | 0.1 | 0.282 | 99 | 1 | 2 |
| 65 | 8.8 | 0.4 | 1.468 | 107 | 20 | 3 |
| 64 | 10.1 | 0.2 | 0.074 | 93 | 7 | 3 |
| 62 | 9.8 | 0.2 | 0.147 | 86 | 9 | 3 |
| 60 | 9.8 | 0.1 | 0.158 | 93 | 6 | 2 |
| 61 | 9.5 | 0.1 | 0.355 | 109 | 36 | 2 |

TABLE B
EC₅₀ (potency) and Emax (efficacy) of the comparative
peptide with respect to human GLP-2R.

| Compound | logEC₅₀ | | EC₅₀ (nM) | Efficacy | | |
|---|---|---|---|---|---|---|
| no. | GLP-2R | SD | GLP-2R | GLP-2R | SD | n= |
| 66 | N/A | | | | | |
| 63 | 8.0 | 0.4 | 11.2 | 105 | 13 | 2 |
| 65 | 8.2 | 0.3 | 6.8 | 77 | 6 | 3 |
| 64 | 8.8 | 0.1 | 1.5 | 75 | 13 | 3 |
| 62 | 9.0 | 0.1 | 1.1 | 83 | 8 | 3 |
| 60 | 7.2 | 0.1 | 70.8 | 90 | 9 | 2 |
| 61 | 7.7 | 0.1 | 20.0 | 96 | 12 | 2 |

N/A: Not available

TABLE C
EC₅₀ (potency) and Emax (efficacy) of the comparative
peptide with respect to human GLP-1R.

| Compound | logEC₅₀ | | EC₅₀ (nM) | Efficacy | | |
|---|---|---|---|---|---|---|
| no. | GLP-1R | SD | GLP-1R | GLP-1R | SD | n= |
| 66 | 7.3 | 0.3 | 50.12 | 81 | 29 | 3 |
| 63 | 8.6 | 0.4 | 2.82 | 84 | 28 | 2 |
| 65 | 8.0 | 0.2 | 10.00 | 76 | 31 | 3 |
| 64 | 9.3 | 0.2 | 0.54 | 85 | 16 | 3 |
| 62 | 8.9 | 0.3 | 1.26 | 85 | 12 | 3 |
| 60 | 8.4 | 0.1 | 4.47 | 105 | 19 | 2 |
| 61 | 8.2 | 0.1 | 7.08 | 98 | 1 | 2 |

### Example 3

### Methods

HEK293 cells were cultured at 10% CO₂ and 37° C. in DMEM-GlutaMAX™-I supplemented with 10% FBS, 180 units/mL penicillin and 45 g/mL streptomycin. These cells were transfected using PEI for the beta-arrestin recruitment assay as described in detail in (van der Velden et al., 2021).

HEK293 cells were transiently transfected with human GIPR, the donor Rluc8-Arrestin-3-Sp1 (beta-arrestin 2 recruitment), the acceptor mem-linker-citrine-SH3, and the GPCR kinase 2 (GRK2). Two days following transfection, the cells were washed with PBS and resuspended in PBS with 5 mM glucose. Then, 85 μL of the cell suspension solution was added to each well of a white 96-well plate followed by the addition of PBS with 5 μM coelenterazine-h. Following 10 min incubation, increasing concentrations of ligands were added and incubated for an additional 30 min. Luminescence was measured with the Berthold Technologies Mithras Multilabel Reader (Rluc8 at 485±40 nm and YFP at 530±25 nm). The potencies (EC₅₀ values) were determined by nonlinear regression using GraphPad Prism 9 (San Diego, California, USA). Sigmoid curves were fitted logistically. The efficacies (Eₘₐₓ values) were determined by normalization to the highest concentration applied of the endogenous ligand (GIP) for the GIPR. The AUC values for the sigmoid curves up to the concentration of 100 nM were likewise calculated.

### Results

The tested dual agonists did all result in less beta-arrestin 2 recruitment to the human GIPR than native GIP and some of them did not recruit beta-arrestin 2 at all.

TABLE 4
EC₅₀ (potency), Emax (Efficacy) and AUC (area under
the curve) of the tested peptide dual agonists
of GIPR and GLP-2R with respect to human GIPR.

| Compound no. | pEC₅₀ ± SD | Emax ± SD (%) | AUC ± SD | n |
|---|---|---|---|---|
| hGIP | 9.2 ± 0.1 | 100 ± 4 | 223 ± 16 | 3 |
| 11 | 8.4 ± 0.3 | 41 ± 6 | 61 ± 14 | 3 |
| 24 | 9.1 ± 0.6 | 57 ± 16 | 117 ± 31 | 3 |
| 25 | 8.9 ± 0.6 | 23 ± 7 | 42 ± 15 | 3 |
| 31 | NA | NA | 0 | 3 |
| 33 | 8.4 ± 0.9 | 9 ± 5 | 23 ± 12 | 3 |
| 35 | 8.5 ± 0.3 | 34 ± 7 | 58 ± 14 | 3 |
| 37 | 8.0 ± 4.3 | 15 ± 41 | 0 | 3 |

NA = no activity.

### Example 4

### Methods

HEK293A cells were cultured at 5% CO₂ and 37° C. in DMEM-GlutaMAX™-I supplemented with 10% FBS, 180 units/mL penicillin and 45 g/mL streptomycin. These cells were transfected using Lipofectamine 2000 from Thermo Fischer Scientific, Massachusetts, USA according to the manufacturer's instructions for the receptor internalization assay.

HEK293A cells transiently expressing human SNAP-GIPR were seeded in white 384-well plates on the day of transfection at a density of 15.000 cells/well. The following day the media was removed and new, fresh culture media was added. On day three, the assay was run. First, the cells were labeled with 100 nM Tag-Lite SNAP-Lumi4-Tb (donor) in OptiMEM for 60 min at 37° C. Next, the cells were washed four times with internalization buffer (HBBS supplemented with 1 mM CaCl₂), 1 mM MgCl2 and 20 mM HEPES) followed by addition of 100 μM preheated fluorescein-O′-acetic acid (acceptor). The plate was placed in a 37° C. incubator for 5 minutes prior to ligand stimulation to adjust the temperature before reading the plate. Then the cells were stimulated with 37° C. preheated ligand solution and internalization was measured every third minute for 60 minutes at 37° C. in PerkinElmer™ EnVision 2014 Multilabel Reader (PerkinElmer, Waltham, MA). The AUC values for the sigmoid curves up to the concentration of 1 μM were likewise calculated.

### Results

The tested dual agonists did all induce less GIPR internalization than native GIP. The compounds that did not recruit beta-arrestin 2 to the GIPR at all did not either induce in any GIPR internalization.

TABLE 5
EC₅₀ (potency), Emax (Efficacy) and AUC (area under
the curve) of the tested peptide dual agonists
of GIPR and GLP-2R with respect to human GIPR.

| | pEC₅₀ ± SD | Emax ± SD (%) | AUC ± SD | n |
|---|---|---|---|---|
| hGIP | 8.0 ± 0.04 | 104 ± 2 | 204 ± 3 | 3 |
| 11 | 6.8 ± 0.4 | 24 ± 6 | 19 ± 2 | 3 |
| 24 | 7.7 ± 0.1 | 68 ± 3 | 113 ± 4 | 3 |
| 25 | 7.2 ± 0.1 | 46 ± 2 | 57 ± 2 | 3 |
| 31 | NA | NA | 0 | 3 |
| 33 | 7.0 ± 0.2 | 25 ± 3 | 27 ± 2 | 2 |
| 36 | 6.7 ± 0.1 | 58 ± 8 | 47 ± 1 | 2 |
| 37 | 6.4 ± 0.3 | 24 ± 5 | 15 ± 4 | 3 |

NA = no activity.

### Example 5

### Methods

COS-7 cells were cultured at 10% CO₂ and 37° C. in Dulbecco's Modified Eagle Medium (DMEM) 1885 supplemented with 10% fetal bovine serum (FBS), 2 mM glutamine, 180 units/ml penicillin, and 45 g/ml streptomycin. Transient transfection of COS-7 cells was performed using the calcium phosphate precipitation method as previously described (van der Velden et al., 2021) prior to experiment.

Transiently transfected COS-7 cells expressing human GIPR were seeded in clear 96-well plates the day after transfection with a density of 7.500 cells/well. The number of cells added per well was adjusted to ensure 5-10% specific binding of the radioligand, ¹²⁵I-GIP (1-42) (NEX402010UC, PerkinElmer, Waltham, MA). The following day, the media of the cells were changed to DMEM supplemented 2% FBS and preincubated either with or without 10 nM ligand for 90 min. The cells were then washed twice in binding buffer (HEPES+0.1% casein) and then incubated for 3 h at 4° C. using 15-40 pM of ¹²⁵I-GIP (1-42). After incubation, the cells were washed twice in 100 μl per well ice-cold binding buffer and lysed using 175 μl per well of 200 mM NaOH with 1% SDS for 30 min. The samples were analyzed by the Wizard Gamme Counter (PerkinElmer, Waltham, MA). Data was analyzed by comparing the counts measured from the ligand preincubated and non-preincubated cells.

### Results

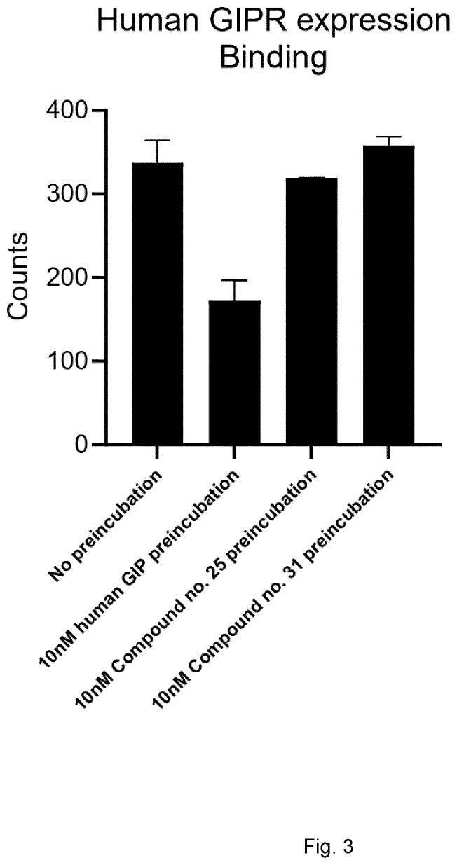

As shown in FIG. 3, preincubation with GIP results in a downregulation of the GIPR from the cell surface which is not observed for any of the two peptide dual agonists tested.

### Example 6

### Method

Sprague Dawley female rats, age 10 w (Janvier Labs, France), n=4, were fasted from 06:00 until after final blood sample. A baseline blood sample was taken just before subcutaneous administration of compound at 10:00. Compounds were dissolved in 50 mM phosphate buffer, pH 7.5+0.1% Haemaccel. Dose: Compound no. 11 4248.5 μg/kg.

Blood samples were taken at 10:00, 14:00, 18:00 and 22:00. Plasma was obtained within 20 min after blood sampling and stored at −20C.

CTX and P1NP were measured using RatLaps (CTX-I) EIA and Rat/mouse P1NP EIA (Immunidiagnosticsystems, UK) according to protocols.

Data are presented using GraphPad Prism V9.3.0 as % of baseline as mean+/−SEM and analyzed by 2-way Anova with Sidaks multiple comparisons tests or by unpaired t-test.

### Results

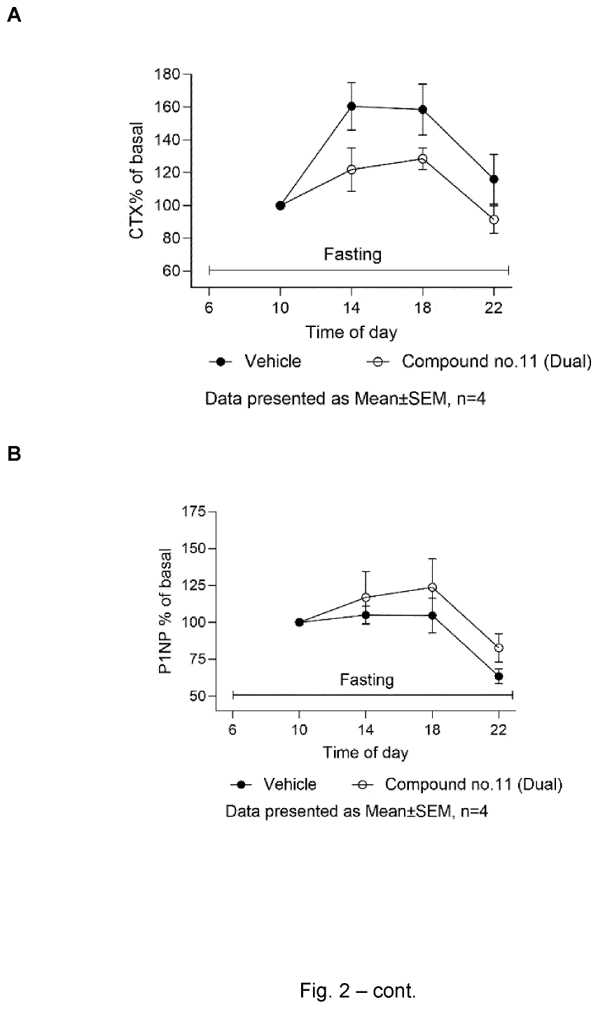

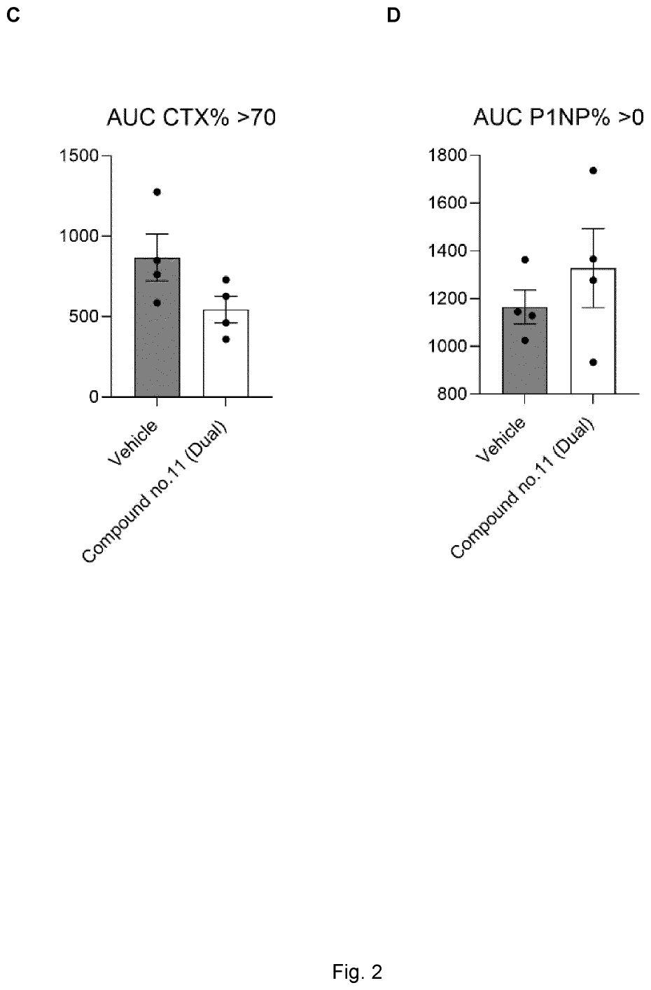

Administration of Compound no. 11 showed a reduced (ns) increase of the resorption marker CTX (time point 14:00, p-value=0.08), see FIG. 2A. The AUC calculated from above lowest measured point (70%) showed a tendency towards a reduction of CTX AUC (p-value=0.10), see FIG. 2C. For the formation marker P1NP, there was an increase (FIG. 2B) at time 14:00 and 18:00 by administration of Compound no. 11.

### Example 7

### Methods

Sprague Dawley male or female rats, weight 200-300 g, n=3, were used for PK studies. Compounds were dissolved in 80% DMSO and further diluted/or directly dissolved in 50 mM phosphate buffer, pH 7.5+0.1% Haemaccel or casein. Dose given was 100 nmol/kg for all SC administrations and 20 nmol/kg or 100 nmol/kg for IV administrations. Blood samples were taken in the timeframe 0-72 H after compound administration. Plasma was obtained and stored at −20° C. until evaluation by LC-MS or LC-MS/MS.

### Results

TABLE 6
Half-life (T_{1/2}) measurements of selected peptide
dual agonists administered to rats either subcutaneously
(SC) or intravenously (IV).

T_{1/2} (h)

| Compound | SC | IV |
|---|---|---|
| no. | administration | administration |
| 24 | 2.06 | ND |
| 25 | 12 | 11.2 |
| 31 | 2.65 | ND |
| 33 | 6.02 | ND |
| 35 | 3.68 | ND |
| 37 | 5.98 | ND |
| 40 | 5.24 | ND |
| 44 | 3.25 | ND |

ND = not determined.

### Example 8

### Methods

COS-7 cells were cultured at 10% CO₂ and 37° C. in Dulbecco's Modified Eagle Medium (DMEM) 1885 supplemented with 10% fetal bovine serum (FBS), 2 mM glutamine, 180 units/ml penicillin, and 45 g/ml streptomycin. Transient transfection of COS-7 cells was performed using the calcium phosphate precipitation method as previously described (van der Velden et al., 2021) prior to experiment.

Transiently transfected COS-7 cells expressing either rat GIPR or GLP-2R, or macaques GIPR or GLP-2R were seeded in white 96-well plates the day after transfection with a density of 35.000 cells/well. The next day the assay was initiated by washing with HEPES buffered saline (HBS) and followed by an incubation step with assay buffer (HBS added 1 mM isobutyl-1-methylxanthine (IBMX)) for 30 min at 37° C. The ligands were then added and incubated for an additional 30 min at 37° C. After ligand incubation, the HitHunter® CAMP assay (Eurofins DiscoverX, Fremont, 150 USA) was carried out according to the manufacturer's instructions. Luminescence was measured by PerkinElmer™ EnVision 2014 Multilabel Reader (PerkinElmer, Waltham, MA). The potencies (EC₅₀ values) were determined by nonlinear regression using GraphPad Prism 9 (San Diego, California, USA). Sigmoid curves were fitted logistically. The efficacies (Eₘₐₓ values) were determined by normalization to the highest concentration applied of the endogenous ligand for each receptor.

### Results

The results in Tables 5 and 6 below indicate that the tested peptide dual agonists, although developed based on human receptors and peptide ligands, have good efficacy towards the GIPR and GLP-2R of rats and macaques as well, thus indicating that their efficacy in medical treatment can be tested in animal models.

TABLE 5
EC₅₀ (potency) and Emax (efficacy) of the tested peptide dual agonists
of GIPR and GLP-2R with respect to rat GIPR and rat GLP-2R.

| | Rat receptors | | | |
|---|---|---|---|---|
| Compound | rGIPR | | rGLP-2R | |
| number | pEC50 | Efficacy (%) | pEC50 | Efficacy (%) |
| 9 | 9.5 | 74.3 | 7.9 | 82.0 |
| 11 | 8.6 | 107.0 | 8.4 | 104.0 |
| 13 | 7.5 | 69.7 | 8.1 | 83.7 |
| 14 | 8.8 | 98.3 | 8.4 | 93.3 |
| 24 | 8.6 | 96.3 | 8.3 | 76.3 |
| 25 | 8.0 | 71.7 | 8.3 | 65.0 |
| 31 | 8.5 | 85.0 | 8.9 | 86.3 |
| 33 | 8.6 | 98.7 | 8.5 | 96.3 |
| 35 | 8.3 | 86.3 | 8.5 | 87.3 |
| 37 | 8.8 | 91.0 | 8.9 | 83.3 |
| 44 | 8.4 | 87.3 | 8.1 | 90.0 |
| 53 | 8.7 | 77.3 | 7.5 | 18.0 |
| 54 | 7.5 | 110.3 | 9.3 | 108.0 |

TABLE 6
EC₅₀ (potency) and Emax (efficacy) of the tested
peptide dual agonists of GIPR and GLP-2R with
respect to macaque GIPR and macaque GLP-2R.

| | Macaca receptors | | | |
|---|---|---|---|---|
| Compound | maGIPR | | maGLP-2R | |
| number | pEC50 | Efficacy (%) | pEC50 | Efficacy (%) |
| 5 | 9.6 | 111.5 | 8.8 | 120.5 |
| 11 | 9.0 | 92.5 | 8.8 | 83.3 |
| 14 | 9.4 | 99.3 | 8.7 | 97.3 |
| 24 | 9.4 | 99.3 | 9.4 | 93.9 |
| 25 | 8.9 | 100.2 | 9.1 | 89.5 |
| 31 | 8.7 | 97.5 | 8.6 | 87.7 |
| 33 | 9.1 | 104.8 | 8.5 | 95.3 |
| 35 | 9.3 | 87.3 | 9.4 | 91.7 |
| 37 | 8.9 | 91.3 | 8.9 | 91.0 |
| 44 | 8.4 | 73.0 | 8.7 | 98.4 |
| 53 | 8.6 | 99.8 | 7.9 | 77.0 |
| 54 | 7.7 | 71.8 | 8.2 | 85.6 |

### Example 9. Sequence Overview of the Dual Agonists of the Present Disclosure

K(γGlu-C16) as provided herein should be read as fatty acid C16 is attached to the side chain amino group of a Lysine via a yGlu linker. Thus, the yGlu linker is attached directly to the amino group on the side chain of a Lysine, and the fatty acid is attached directly to the yGlu. The same applies to the other fatty acid and linkers listed in parenthesis herein below.

Compound no. 9 SEQ ID NO: 8
H-Aib-DG-K(γGlu-C16)-FISDYSTILDNLAARDFINWLIQTKITK-OH
Compound no. 10 SEQ ID NO: 9
H-Aib-DGTF-K(γGlu-C16)-SDYSTILDNLAARDFINWLIQTKITK-OH
Compound no. 11 SEQ ID NO: 10
H-Aib-DGTFISDY-K(γGlu-C16)-TILDNLAARDFINWLIQTKITK-OH
Compound no. 12 SEQ ID NO: 11
H-Aib-DGTFISDYS-K(γGlu-C16)-ILDNLAARDFINWLIQTKITK-OH
Compound no. 13 SEQ ID NO: 12
H-Aib-DGTFISDYSTIL-K(γGlu-C16)-NLAARDFINWLIQTKITK-OH
Compound no. 14 SEQ ID NO: 13
H-Aib-DGTFISDY-K(γGlu-C16)-TILDNLAARDFINWLIQTKGKKNDWKHNITQ-NH2
Compound no. 24 SEQ ID NO: 21
H-Aib-DG-K(γGlu-C10)-FISDYST-Aib-LDNLAARDFINWLIQTKITK-OH
Compound no. 25 SEQ ID NO: 21
H-Aib-DG-K(γGlu-C16 diacid)-FISDYST-Aib-LDNLAARDFINWLIQTKITK-OH
Compound no. 26 SEQ ID NO: 8 B
H-Aib-DG-K(γGlu- γGlu-C10)-FISDYSTILDNLAARDFINWLIQTKITK-OH
Compound no. 27 SEQ ID NO: 22
H-Aib-EG-K(γGlu-C16)-FISDYST-Aib-LDNLAARDFINWLIQTKITK-OH
Compound no. 28 SEQ ID NO: 21
H-Aib-DG-K(γGlu- γGlu-C16)-FISDYST-Aib-LDNLAARDFINWLIQTKITK-OH
Compound no. 29 SEQ ID NO: 23
H-Aib-DGTFISDY-K(γGlu-C16)-T-Aib-LDNLAARDFINWLIQTKITK-OH
Compound no. 30 SEQ ID NO: 25
H-Aib-DGTFISDY-K(γGlu-C10)-T-Aib-LDNLAARDFINWLIQTKITD-OH
Compound no. 31 SEQ ID NO: 26 B
H-Aib-DGTFISDY-K(γGlu-C10)-TILDKLAARDFIEWLIATKITK-OH
Compound no. 32 SEQ ID NO: 27
H-Aib-DGTFISDY-K(γGlu-C16)-TILDNLAARDFINWLIETKITK-OH
Compound no. 33 SEQ ID NO: 28
H-Aib-DGTFISDY-K(γGlu-C16)-T-Aib-LDALAARDFIEWLIQTKITK-OH
Compound no. 34 SEQ ID NO: 24
Ac-H-Aib-DGTFISDY-K(γGlu-C16)-T-Aib-LDNLAARDFINWLIQTKITK-OH
Compound no. 35 SEQ ID NO: 29
H-Aib-DGTFISDYS-K(γGlu-C16)-Aib-LDNLAARDFINWLIQTKITK-OH
Compound no. 36 SEQ ID NO: 30
Ac-H-Aib-DGTFISDYS-K(γGlu-C16)-Aib-LDNLAARDFINWLIQTKITK-OH
Compound no. 37 SEQ ID NO: 31
H-Aib-DGTFISDY-K(γGlu-C16)-TAib-LDNLAARDFINWLIQTKITKNDWKHNITQ-OH
Compound no. 38 SEQ ID NO: 32
Ac-H-Aib-DGTFISDY-K(γGlu-C16)-T-Aib-LDNLAARDFINWLIQTKITKNDWKHNITQ-OH
Compound no. 54 SEQ ID NO: 46
H-Aib-DGTFISDY-K(yglu-C10)-T-Aib-LDNLAARDFINWLIQTKITGNDWKHNITQ-OH
Compound no. 1 SEQ ID NO: 1
(C16)HADGTFISDYSTILDNLAARDFINWLIQTKITD
Compound no. 2 SEQ ID NO: 2
(C16)HADGTFISDYSTILDNLAARDFINWLIQTKIT
Compound no. 45 SEQ ID NO: 38
(C16)HADGTFISDYSTILDNLAAKDFINWLIQTKITK
Compound no. 3 SEQ ID NO: 3
(C10)HADGTFISDYSTILDNLAAKDFINWLIQTKITKNDWKHNITQ
Compound no. 46 SEQ ID NO: 39
(C16)HADGTFISDYSTILDNLAARDFINWLLAQKITK
Compound no. 4 SEQ ID NO: 1
(C16)HADGTFISDYSTILDNLAARDFINWLIQTKITD
Compound no. 5 SEQ ID NO: 4
(C16)H-Aib-DGTFISDYST-Aib-LDNLAARDFINWLIQTKITK-OH
Compound no. 6 SEQ ID NO: 5
(C16)H-Aib-DGTFISDYSTILD-Aib-LAARDFINWLIQTKITK-OH
Compound no. 7 SEQ ID NO: 6
(C16)H-Aib-DGTFISDYSTILDNLAARDFIEWLIQTKITK-OH
Compound no. 8 SEQ ID NO: 7
(C16)H-Aib-DGTFISDYSTILDNLAARDFIEWLIQTKIT-OH
Compound no. 15 SEQ ID NO: 14
(C16)HADGTFISDYSTILELLAARDFINWLIQTKITKNDWKHNITQ
Compound no. 48 SEQ ID NO: 41
(C16)HADGTFISDYSTILDELAARDFINWLIQTKITKNDWKHNITQ
Compound no. 49 SEQ ID NO: 42
(C16)HADGTFISDYSTILDNLAARDFINWLIQTKITKAEWKHAITQ
Compound no. 50 SEQ ID NO: 43
(C16)HADGTFISDYSTILDNLAARDFIEWLIQTKITKNDWKHNITQ
Compound no. 51 SEQ ID NO: 44
(C16)HADGTFISDYSTILDNLAARDFINWLIETKITKNDWKHNITQ
Compound no. 52 SEQ ID NO: 45
(C16)HADGTFISDYSTILDNLAARDFINWLIATKITKNDWKHNITQ
Compound no. 53 SEQ ID NO: 33
(C16)HADGTFISDYSTAibLDNLAARDFINWLIQTKITKNDWKHNITQ
Compound no. 16 SEQ ID NO: 15
(C16)HADGTFISDYSTILDAibLAARDFINWLIQTKITKNDWKHNITQ
Compound no. 17 SEQ ID NO: 4
(C16-diacid)H-Aib-DGTFISDYST-Aib-LDNLAARDFINWLIQTKITK-OH
Compound no. 18 SEQ ID NO: 4
(C10-diacid)H-Aib-DGTFISDYST-Aib-LDNLAARDFINWLIQTKITK-OH
Compound no. 19 SEQ ID NO: 16
(C16-diacid)H-Aib-EGTFISDYST-Aib-LDNLAARDFINWLIQTKITK-OH
Compound no. 20 SEQ ID NO: 17
(C16-diacid)H-Aib-DGTFISDYST-Aib-LDALAARDFIEWLIQTKITK-OH
Compound no. 21 SEQ ID NO: 18
(C16-diacid)H-Aib-EGTFISDYST-Aib-LDKLAARDFINWLIQTKITK-OH
Compound no. 22 SEQ ID NO: 19
(C16-diacid)H-Aib-DGTFISDYST-Aib-LDKLAARDFIEWLIQTKITK-OH
Compound no. 23 SEQ ID NO: 20
(C16-diacid)H-Aib-DGTFISDYST-Aib-LDKLAARDFINWLIETKITK-OH
Compound no. 39 SEQ ID NO: 33
(C16-diacid)HADGTFISDYSTAibLDNLAARDFINWLIQTKITKNDWKHNITQ
Compound no. 40 SEQ ID NO: 34
(C16-diacid)H-Aib-DGTFISDYST-Aib-LDNLAARDFINWLIQTKITKNDWKHNITQ-OH
Compound no. 41 SEQ ID NO: 35
(C16-diacid)H-Aib-EGTFISDYST-Aib-LDALAARDFINWLIQTKITKNDWKHNITQ-OH
Compound no. 42 SEQ ID NO: 36
(C16-diacid)H-Aib-DGTFISDYST-Aib-LDNLAAKDFINWLIQTKITKNDWKHNITQ-OH
Compound no. 43 SEQ ID NO: 37
(C16-diacid)H-Aib-EGTFISDYST-Aib-LDNLAAKDFINWLIQTKITKNDWKHNITQ-OH
Compound no. 44 SEQ ID NO: 4
(C10)H-Aib-DGTFISDYST-Aib-LDNLAARDFINWLIQTKITK-OH
Compound no. 56 SEQ ID NO: 47
HADGTFISDYSTILDKLAAK(C16)DFINWLIQTKITK
Compound no. 57 SEQ ID NO: 48
HADGTFISDYSTILDKLAARDFINWLIQTK(C16)ITK
Compound no. 58 SEQ ID NO: 48
HADGTFISDYSTILDKLAARDFINWLIQTKITK(C16)
Compound no. 60 SEQ ID NO: 49
HADGTFISDYSTILDNLAARDFINWLIQTKGKK(γGlu-C18-diacid)
Compound no. 61 SEQ ID NO: 50
HADGTFISDYSTAibLDNLAARDFINWLIQTKGKKK(γGlu-C18-diacid)
Compound no. 62 SEQ ID NO: 51
HADGTFISDYSTAibLDK(γGlu-C18-diacid) LAARDFINWLIQTKGKK
Compound no. 63 SEQ ID NO: 52
H-Aib-DGTFISDYSTILDNLAARDFINWLIQTKITK(C10)-OH
Compound no. 64 SEQ ID NO: 53
HADGTFISDYSTILDK(γGlu-C18-diacid) LAARDFINWLIQTKGKK
Compound no. 65 SEQ ID NO: 54
H-Aib-DGTFISDYST-Aib-LDNLAARDFINWLIQTKITK(C10)-NH2
Compound no. 66 SEQ ID NO: 55
HGEGSFGSDMSIALDKLAARDFVNWLLQTKITD

### REFERENCES

Van der Velden et al. 2021. GLP-1 Val8: A Biased GLP-1R Agonist with Altered Binding Kinetics and Impaired Release of Pancreatic Hormones in Rats
Sameer et al. Mol Cell Biol. 2014 Oct. 1; 34 (19): 3618-29. doi: 10.1128/MCB.00256-14.

### Items

1. A peptide dual agonist comprising or consisting of the sequence

(SEQ ID NO: 66)
HX₂X₃GX₅FX₇X₈X₉X₁₀X₁₁X₁₂X₁₃X₁₄X₁₅X₁₆LAAX₂₀DFIX₂₄WLIX₂₈TK
X₃₁X₃₂X₃₃-Z,

wherein
X₂ is A, K or Aib,
X₃ is D, K or E,
X₅ is T, K or S,
X₇ is K or I
X₈ is K or S
X₉ is D or K,
X₁₀ is Y or K,
X₁₁ is S, K or N,
X₁₂ is K or T
X₁₃ is A, Aib, K or I,
X₁₄ is L or K,
X₁₅ is K, D or E,
X₁₆ is E, L, Aib, K, A, N
X₂₀ is R or K,
X₂₄ is N or E,
X₂₈ is Q, E or A,
X₃₁ is I, G or omitted,
X₃₂ is T, K or omitted, and
X₃₃ is K, G, D or omitted,
wherein Z is a peptide comprising one or more amino acid residues of GIP(34-42) (NDWKHNITQ; SEQ ID NO: 58),
wherein said peptide is modified by attaching at least one fatty acid molecule at one or more amino acid residues at any one of positions 1 to 15 of SEQ ID NO: 66, and
wherein said peptide is an agonist of GIPR (glucose-dependent insulinotropic polypeptide receptor) and is an agonist of GLP-2R (glucagon-like peptide-2 receptor).

2. The peptide dual agonist according to item 1, wherein said peptide is modified by attaching at least one fatty acid molecule at any one of positions 2 to 15 of SEQ ID NO: 66.

3. The peptide dual agonist according to any one of items 1 or 2, wherein
X₂ is K or Aib,
X₃ is Dor E,
X₅ is T or K,
X₇ is I,
X₁₁ is S or K,
X₁₃ is Aib or I,
X₁₅ is D or E,
X₁₆ E, L, Aib, K, A, N
X₂₄ is N or E,
X₂₈ is Q, E or A,
X₃₁ is I, G or omitted,
X₃₂ is T or omitted.

4. The peptide dual agonist according to item 1, wherein
X₃ is D,
X₇ is I,
X₁₅ is D,
X₃₁ is I, and
X₃₂ is T.

5. The peptide dual agonist according to any one of the preceding items, wherein X₂ is Aib.

6. The peptide dual agonist according to any one of the preceding items, wherein X₁₃ is Aib.

7. The peptide dual agonist according to any one of the preceding items, wherein X₁₆ is A, Aib, L, E.

8. The peptide dual agonist according to any one of the preceding items, wherein X₂ is Aib and X₁₃ is Aib.

9. The peptide dual agonist according to any one of the preceding items, wherein X₂ is Aib and X₁₁ is K.

10. The peptide dual agonist according to any one of the preceding items, wherein X₂ is Aib, X₅ is K and X₁₃ is Aib.

11. The peptide dual agonist according to any one of the preceding items, wherein X₂ is Aib, X₁₁ is K and X₁₃ is Aib.

12. The peptide dual agonist according to any one of the preceding items, wherein X₂ is Aib, X₁₁ is K and X₂₄ is E.

13. The peptide dual agonist according to any one of the preceding items, wherein Z is selected from the group consisting of:
ND, NDW, KDW, NDW, NDK, NDWK, NDWKH, NDWKHN, NDWKHNI, NDWKHNIT, NDWKHNITQ, and AEWKHAITQ or a variant comprising one amino acid substitution.

14. The peptide dual agonist according to any one of the preceding items, wherein Z is selected from the group consisting of:

(SEQ ID NO: )
AEWKHAITQ
and
(SEQ ID NO: )
NDWKHNITQ.

15. The peptide dual agonist according to item 1, wherein said peptide is modified by attaching at least one fatty acid molecule at the N-terminal amino acid residue of SEQ ID NO: 66, such as at the amino acid residue at position 1 of SEQ ID NO: 66.

16. The peptide dual agonist according to any one of the preceding items, wherein X₇ is I, and wherein said peptide is modified by attaching one fatty acid molecule at position 1 of SEQ ID NO: 66, such as at the N-terminus of SEQ ID NO: 66.

17. The peptide dual agonist according to any one of the preceding items, wherein said peptide is selected from the group consisting of

Compound no. 9
SEQ ID NO: 8
H-Aib-DGKFISDYSTILDNLAARDFINWLIQTKITK,
Compound no. 10
SEQ ID NO: 9
H-Aib-DGTFKSDYSTILDNLAARDFINWLIQTKITK,
Compound no. 11
SEQ ID NO: 10
H-Aib-DGTFISDYKTILDNLAARDFINWLIQTKITK,
Compound no. 12
SEQ ID NO: 11
H-Aib-DGTFISDYSKILDNLAARDFINWLIQTKITK,
Compound no. 13
SEQ ID NO: 12
H-Aib-DGTFISDYSTILKNLAARDFINWLIQTKITK,
Compound no. 14
SEQ ID NO: 13
H-Aib-DGTFISDYKTILDNLAARDFINWLIQTKGKKNDWKHNITQ,
Compound no. 24
SEQ ID NO: 21
H-Aib-DGKFISDYST-Aib-LDNLAARDFINWLIQTKITK,
Compound no. 25
SEQ ID NO: 21
H-Aib-DGKFISDYST-Aib-LDNLAARDFINWLIQTKITK,
Compound no. 26
SEQ ID NO: 8
H-Aib-DGKFISDYSTILDNLAARDFINWLIQTKITK,
Compound no. 27
SEQ ID NO: 22
H-Aib-EGKFISDYST-Aib-LDNLAARDFINWLIQTKITK,
Compound no. 28
SEQ ID NO: 21
H-Aib-DGKFISDYST-Aib-LDNLAARDFINWLIQTKITK,
Compound no. 29
SEQ ID NO: 23
H-Aib-DGTFISDYKT-Aib-LDNLAARDFINWLIQTKITK,
Compound no. 30
SEQ ID NO: 25
H-Aib-DGTFISDYKT-Aib-LDNLAARDFINWLIQTKITD,
Compound no. 31
SEQ ID NO: 26
H-Aib-DGTFISDYKTILDKLAARDFIEWLIATKITK,
Compound no. 32
SEQ ID NO: 27
H-Aib-DGTFISDYKTILDNLAARDFINWLIETKITK,
Compound no. 33
SEQ ID NO: 28
H-Aib-DGTFISDYKT-Aib-LDALAARDFIEWLIQTKITK,
Compound no. 34
SEQ ID NO: 24
Ac-H-Aib-DGTFISDYKT-Aib-LDNLAARDFINWLIQTKITK,
Compound no. 35
SEQ ID NO: 29
H-Aib-DGTFISDYSKAib-LDNLAARDFINWLIQTKITK,
Compound no. 36
SEQ ID NO: 30
Ac-H-Aib-DGTFISDYSKAib-LDNLAARDFINWLIQTKITK,
Compound no. 37
SEQ ID NO: 31
H-Aib-DGTFISDYKTAib-LDNLAARDFINWLIQTKITKNDWKHNI
TQ,
Compound no. 38
SEQ ID NO: 32
Ac-H-Aib-DGTFISDYKT-Aib-LDNLAARDFINWLIQTKITKNDWK
HNITQ,
Compound no. 54
SEQ ID NO: 46
H-Aib-DGTFISDYKT-Aib-LDNLAARDFINWLIQTKITGNDWKHNI
TQ,
Compound no. 1
SEQ ID NO: 1
HADGTFISDYSTILDNLAARDFINWLIQTKITD,
Compound no. 2
SEQ ID NO: 2
HADGTFISDYSTILDNLAARDFINWLIQTKIT,
Compound no. 45
SEQ ID NO: 38
HADGTFISDYSTILDNLAAKDFINWLIQTKITK,
Compound no. 3
SEQ ID NO: 3
HADGTFISDYSTILDNLAAKDFINWLIQTKITKNDWKHNITQ,
Compound no. 46
SEQ ID NO: 39
HADGTFISDYSTILDNLAARDFINWLLAQKITK,
Compound no. 4
SEQ ID NO: 1
HADGTFISDYSTILDNLAARDFINWLIQTKITD,
Compound no. 5
SEQ ID NO: 4
H-Aib-DGTFISDYST-Aib-LDNLAARDFINWLIQTKITK,
Compound no. 6
SEQ ID NO: 5
H-Aib-DGTFISDYSTILD-Aib-LAARDFINWLIQTKITK,
Compound no. 7
SEQ ID NO: 6
H-Aib-DGTFISDYSTILDNLAARDFIEWLIQTKITK,
Compound no. 8
SEQ ID NO: 7
H-Aib-DGTFISDYSTILDNLAARDFIEWLIQTKIT,
Compound no. 15
SEQ ID NO: 14
HADGTFISDYSTILELLAARDFINWLIQTKITKNDWKHNITQ,
Compound no. 48
SEQ ID NO: 41
HADGTFISDYSTILDELAARDFINWLIQTKITKNDWKHNITQ,
Compound no. 49
SEQ ID NO: 42
HADGTFISDYSTILDNLAARDFINWLIQTKITKAEWKHAITQ,
Compound no. 50
SEQ ID NO: 43
HADGTFISDYSTILDNLAARDFIEWLIQTKITKNDWKHNITQ,
Compound no. 51
SEQ ID NO: 44
HADGTFISDYSTILDNLAARDFINWLIETKITKNDWKHNITQ,
Compound no. 52
SEQ ID NO: 45
HADGTFISDYSTILDNLAARDFINWLIATKITKNDWKHNITQ,
Compound no. 53
SEQ ID NO: 33
HADGTFISDYSTAibLDNLAARDFINWLIQTKITKNDWKHNITQ,
Compound no. 16
SEQ ID NO: 15
HADGTFISDYSTILDAibLAARDFINWLIQTKITKNDWKHNITQ,
Compound no. 17
SEQ ID NO: 4
H-Aib-DGTFISDYST-Aib-LDNLAARDFINWLIQTKITK,
Compound no. 19
SEQ ID NO: 16
H-Aib-EGTFISDYST-Aib-LDNLAARDFINWLIQTKITK,
Compound no. 20
SEQ ID NO: 17
H-Aib-DGTFISDYST-Aib-LDALAARDFIEWLIQTKITK,
Compound no. 21
SEQ ID NO: 18
H-Aib-EGTFISDYST-Aib-LDKLAARDFINWLIQTKITK,
Compound no. 22
SEQ ID NO: 19
H-Aib-DGTFISDYST-Aib-LDKLAARDFIEWLIQTKITK,
Compound no. 23
SEQ ID NO: 20
H-Aib-DGTFISDYST-Aib-LDKLAARDFINWLIETKITK,
Compound no. 39
SEQ ID NO: 33
HADGTFISDYSTAibLDNLAARDFINWLIQTKITKNDWKHNITQ,
Compound no. 40
SEQ ID NO: 34
H-Aib-DGTFISDYST-Aib-LDNLAARDFINWLIQTKITKNDWKHNI
TQ,
Compound no. 41
SEQ ID NO: 35
H-Aib-EGTFISDYST-Aib-LDALAARDFINWLIQTKITKNDWKHNI
TQ,
Compound no. 42
SEQ ID NO: 36
H-Aib-DGTFISDYST-Aib-LDNLAAKDFINWLIQTKITKNDWKHNI
TQ,
Compound no. 43
SEQ ID NO: 37
H-Aib-EGTFISDYST-Aib-LDNLAAKDFINWLIQTKITKNDWKHNI
TQ,
and
Compound no. 44
SEQ ID NO: 4
H-Aib-DGTFISDYST-Aib-LDNLAARDFINWLIQTKITK.

18. The peptide dual agonist according to any one of the preceding items, wherein said peptide is selected from the group consisting of

SEQ ID NO: 4
H-Aib-DGTFISDYST-Aib-LDNLAARDFINWLIQTKITK,
SEQ ID NO: 5
H-Aib-DGTFISDYSTILD-Aib-LAARDFINWLIQTKITK,
SEQ ID NO: 8
H-Aib-DGKFISDYSTILDNLAARDFINWLIQTKITK,
SEQ ID NO: 10
H-Aib-DGTFISDYKTILDNLAARDFINWLIQTKITK,
SEQ ID NO: 11
H-Aib-DGTFISDYSKILDNLAARDFINWLIQTKITK,
SEQ ID NO: 13
H-Aib-DGTFISDY-KTILDNLAARDFINWLIQTKGKKNDWKHNITQ,
SEQ ID NO: 4
H-Aib-DGTFISDYST-Aib-LDNLAARDFINWLIQTKITK,
SEQ ID NO: 16
H-Aib-EGTFISDYST-Aib-LDNLAARDFINWLIQTKITK,
SEQ ID NO: 17
H-Aib-DGTFISDYST-Aib-LDALAARDFIEWLIQTKITK,
SEQ ID NO: 19
H-Aib-DGTFISDYST-Aib-LDKLAARDFIEWLIQTKITK,
SEQ ID NO: 21
H-Aib-DGKFISDYST-Aib-LDNLAARDFINWLIQTKITK,
SEQ ID NO: 23
H-Aib-DGTFISDYKT-Aib-LDNLAARDFINWLIQTKITK,
SEQ ID NO: 28
H-Aib-DGTFISDYKT-Aib-LDALAARDFIEWLIQTKITK,
SEQ ID NO: 31
H-Aib-DGTFISDY-KTAib-LDNLAARDFINWLIQTKITKNDWKHNI
TQ,
SEQ ID NO: 34
H-Aib-DGTFISDYST-Aib-LDNLAARDFINWLIQTKITKNDWKHNI
TQ,
SEQ ID NO: 35
H-Aib-EGTFISDYST-Aib-LDALAARDFINWLIQTKITKNDWKHNI
TQ,
SEQ ID NO: 36
H-Aib-DGTFISDYST-Aib-LDNLAAKDFINWLIQTKITKNDWKHNI
TQ,
SEQ ID NO: 4
H-Aib-DGTFISDYST-Aib-LDNLAARDFINWLIQTKITK,
and
SEQ ID NO: 26
H-Aib-DGTFISDYKTILDKLAARDFIEWLIATKITK.

19. A peptide dual agonist comprising or consisting of an amino acid sequence selected from the group consisting of:

Compound no. 9
SEQ ID NO: 8
H-Aib-DG-KFISDYSTILDNLAARDFINWLIQTKITK,
Compound no. 10
SEQ ID NO: 9
H-Aib-DGTF-KSDYSTILDNLAARDFINWLIQTKITK,
Compound no. 11
SEQ ID NO: 10
H-Aib-DGTFISDY-KTILDNLAARDFINWLIQTKITK,
SEQ ID NO: 11
H-Aib-DGTFISDYS-KILDNLAARDFINWLIQTKITK,
SEQ ID NO: 12
H-Aib-DGTFISDYSTIL-KNLAARDFINWLIQTKITK,
SEQ ID NO: 13
H-Aib-DGTFISDY-KTILDNLAARDFINWLIQTKGKKNDWKHNITQ,
SEQ ID NO: 21
H-Aib-DG-KFISDYST-Aib-LDNLAARDFINWLIQTKITK,
SEQ ID NO: 22
H-Aib-EG-KFISDYST-Aib-LDNLAARDFINWLIQTKITK,
SEQ ID NO: 23
H-Aib-DGTFISDY-KT-Aib-LDNLAARDFINWLIQTKITK,
SEQ ID NO: 25
H-Aib-DGTFISDY-KT-Aib-LDNLAARDFINWLIQTKITD,
SEQ ID NO: 26
H-Aib-DGTFISDY-KTILDKLAARDFIEWLIATKITK,
SEQ ID NO: 27
H-Aib-DGTFISDY-KTILDNLAARDFINWLIETKITK,
SEQ ID NO: 28
H-Aib-DGTFISDY-KT-Aib-LDALAARDFIEWLIQTKITK,
SEQ ID NO: 24
Ac-H-Aib-DGTFISDY-KT-Aib-LDNLAARDFINWLIQTKITK,
SEQ ID NO: 29
H-Aib-DGTFISDYS-KAib-LDNLAARDFINWLIQTKITK,
SEQ ID NO: 30
Ac-H-Aib-DGTFISDYS-KAib-LDNLAARDFINWLIQTKITK,
SEQ ID NO: 31
H-Aib-DGTFISDY-KTAib-LDNLAARDFINWLIQTKITKNDWKHNI
TQ,
SEQ ID NO: 32
Ac-H-Aib-DGTFISDY-KT-Aib-LDNLAARDFINWLIQTKITKNDW
KHNITQ,
SEQ ID NO: 46
H-Aib-DGTFISDY-KT-Aib-LDNLAARDFINWLIQTKITGNDWKHN
ITQ,
SEQ ID NO: 5
H-Aib-DGTFISDYSTILD-Aib-LAARDFINWLIQTKITK,
SEQ ID NO: 6
H-Aib-DGTFISDYSTILDNLAARDFIEWLIQTKITK,
SEQ ID NO: 7
H-Aib-DGTFISDYSTILDNLAARDFIEWLIQTKIT,
SEQ ID NO: 4
H-Aib-DGTFISDYST-Aib-LDNLAARDFINWLIQTKITK,
SEQ ID NO: 16
H-Aib-EGTFISDYST-Aib-LDNLAARDFINWLIQTKITK,
SEQ ID NO: 17
H-Aib-DGTFISDYST-Aib-LDALAARDFIEWLIQTKITK,
SEQ ID NO: 18
H-Aib-EGTFISDYST-Aib-LDKLAARDFINWLIQTKITK,
SEQ ID NO: 19
H-Aib-DGTFISDYST-Aib-LDKLAARDFIEWLIQTKITK,
SEQ ID NO: 20
H-Aib-DGTFISDYST-Aib-LDKLAARDFINWLIETKITK,
SEQ ID NO: 34
H-Aib-DGTFISDYST-Aib-LDNLAARDFINWLIQTKITKNDWKHNI
TQ,
SEQ ID NO: 35
H-Aib-EGTFISDYST-Aib-LDALAARDFINWLIQTKITKNDWKHNI
TQ,
SEQ ID NO: 36
H-Aib-DGTFISDYST-Aib-LDNLAAKDFINWLIQTKITKNDWKHNI
TQ,
SEQ ID NO: 37
H-Aib-EGTFISDYST-Aib-LDNLAAKDFINWLIQTKITKNDWKHNI
TQ,
and
SEQ ID NO: 4
H-Aib-DGTFISDYST-Aib-LDNLAARDFINWLIQTKITK,

or a functional variant thereof, wherein said functional variant comprises 1 to 3 individual amino acid substitutions with the proviso that the amino acid residue at position 2 of said functional variant is Aib,
the amino acid residue at position 6 of said functional variant is Phenylalanine (F),
the amino acid residue at position 22 of said functional variant is Phenylalanine (F),
the amino acid residue at position 25 of said functional variant is Tryptophan (W), and
the amino acid residue at position 26 of said functional variant is Leucine (L),
wherein said peptide is modified by attaching at least one fatty acid molecule at one or more amino acid residues at any one of positions 1 to 15 of the above sequences, and
wherein said peptide is an agonist of GIPR (glucose-dependent insulinotropic polypeptide receptor) and is an agonist of GLP-2R (glucagon-like peptide-2 receptor).

20. The peptide dual agonist according to any one of the preceding items, wherein said peptide is C-terminally amidated (—NH₂) or wherein the C-terminus is a carboxylic acid.

21. The peptide dual agonist according to any one of the preceding items, wherein said peptide is modified by attaching at least one fatty acid molecule at a Lysine (K) at any one of positions 2 to 15 of SEQ ID NO: 66 or 67.

22. The peptide dual agonist according to any one of the preceding items, wherein said peptide is modified by attaching at least one fatty acid molecule at position 2, position 3, position 4, position 5, position 6, position 7, position 8, position 9, position 10, position 11, position 12, position 13, position 14, or position 15 of SEQ ID NO: 66 or 67.

23. The peptide dual agonist according to any one of the preceding items, wherein said peptide is modified by attaching at least one fatty acid molecule at position 2, position 3, position 4, position 5, position 6, position 7, position 8, position 9, position 10, position 11 or position 12 of SEQ ID NO: 66 or 67.

24. The peptide dual agonist according to any one of the preceding items, wherein said peptide is modified by attaching at least one fatty acid molecule at position 2, position 3, position 4, position 5, position 6, position 7, position 8, position 9, position 10 or position 11 of SEQ ID NO: 66 or 67.

25. The peptide dual agonist according to any one of the preceding items, wherein said peptide is modified by attaching at least one fatty acid molecule at position 5, position 6, position 7, position 8, position 9, position 10, position 11, position 12, position 13, position 14, or position 15 of SEQ ID NO: 66 or 67.

26. The peptide dual agonist according to any one of the preceding items, wherein said peptide is modified by attaching at least one fatty acid molecule at position 5, position 6, position 7, position 8, position 9, position 10, position 11 or position 12 of SEQ ID NO: 66 or 67.

27. The peptide dual agonist according to any one of the preceding items, wherein said peptide is modified by attaching at least one fatty acid molecule at any one of positions 5, 7, 8, 11, 12, 15 of SEQ ID NO: 66 or 67.

28. The peptide dual agonist according to any one of the preceding items, wherein said peptide is modified by attaching at least one fatty acid molecule at any one of positions 5, 7, 8, 11 of SEQ ID NO: 66 or 67.

29. The peptide dual agonist according to any one of the preceding items, wherein X₁₃ is Aib, and wherein said peptide is modified by attaching at least one fatty acid molecule at any one of positions 5 or 11 of SEQ ID NO: 66 or 67.

30. The peptide dual agonist according to any one of the preceding items, wherein said fatty acid molecule is a straight-chain fatty acid.

31. The peptide dual agonist according to any one of the preceding items, wherein said fatty acid molecule is a branched fatty acid.

32. The peptide dual agonist according to any one of the preceding items, wherein said fatty acid molecule is a monoacyl fatty acid molecule, comprising one fatty acid.

33. The peptide dual agonist according to any one of the preceding items, wherein said fatty acid molecule is a diacyl fatty acid molecule.

34. The peptide dual agonist according to any one of the preceding items, wherein said fatty acid molecule comprises an acyl group selected from the group consisting of CH₃(CH₂)₈CO— (capriyl, C10), CH₃(CH₂)₁₀CO— (lauryl, C12), CH₃(CH₂)₁₂CO— (myristoyl, C14), CH₃(CH₂)₁₄CO— (palmitoyl, C16), CH₃(CH₂)₁₆CO— (stearyl, C18) and CH₃(CH₂)₁₈CO— (arachidyl, C20).

35. The peptide dual agonist according to any one of the preceding items, wherein said fatty acid molecule comprises two acyl groups individually selected from the group consisting of HOOC—CH₃(CH₂)₈CO— (decanoyl, C10), HOOC—CH₃(CH₂)₁₀CO— (dodecanoyl, C12), HOOC—CH₃(CH₂)₁₂CO— (1-tetradecanoyl, C14), HOOC—CH₃(CH₂)₁₄CO— (hexadecanoyl, C16), HOOC—CH₃(CH₂)₁₅CO— (15-carboxy-pentadecanoyl, C17), HOOC—CH₃(CH₂)₁₆CO— (octadecanoyl, C18), HOOC—CH₃(CH₂)₁₇CO— (17-carboxy-heptadecanoyl, C19), HOOC—CH₃(CH₂)₁₈CO— (eicosanoyl, C20), and HOOC—CH₃(CH₂)₁₉CO— (19-carboxy-nonadecanoyl, C21).

36. The peptide dual agonist according to any one of the preceding items, wherein said fatty acid molecule is attached to an amino acid residue via a spacer.

37. The peptide dual agonist according to any one of the preceding items, wherein said spacer comprises yGlu.

38. The peptide dual agonist according to any one of the preceding items, wherein said spacer comprises or consists of yGlu or yGlu-yGlu.

39. The peptide dual agonist according to any one of the preceding items, wherein said peptide
a. activates the human GIPR with an efficacy (Eₘₐₓ values) which is at least 65% of the efficacy by which native human GIP activates the human GIPR, such as at least 70%, such as at least 75%, such as at least 80%, such as at least 85%, such as at least 90%, such as at least 95% of the efficacy by which native human GIP activates the human GIPR; and
b. activates the human GLP2-R with an efficacy (Eₘₐₓ values) which is at least 65% of the efficacy by which native human GLP-2 activates the human GLP-2R, such as at least 70%, such as at least 75%, such as at least 80%, such as at least 85%, such as at least 90%, such as at least 95% of the efficacy by which native human GLP-2 activates the human GLP-2R.

40. The peptide dual agonist according to any one of the preceding items, wherein said peptide activates the human GLP-1R with an efficacy (Eₘₐₓ values) which is at the most 80% of the efficacy by which native human GLP-1 activates the human GLP-1R, such as an efficacy (Eₘₐₓ values) which is at the most 70% of the efficacy by which native human GLP-1 activates the human GLP-1R, such as an efficacy (Eₘₐₓ values) which is at the most 60% of the efficacy by which native human GLP-1 activates the human GLP-1R.

41. The peptide dual agonist according to any one of the preceding items, wherein said peptide
a. has an EC₅₀ towards the human GIPR of 10 nM or less; and
b. has an EC₅₀ towards the human GLP-2R of 50 nM or less.

42. The peptide dual agonist according to any one of the preceding items, wherein said peptide has an EC₅₀ towards the human GLP-1R of 10 nM or more.

43. The peptide dual agonist according to any one of the preceding items, wherein said peptide has an EC₅₀ towards the human GIPR that is at least 10 times lower than its EC₅₀ towards human GLP-1R.

44. The peptide dual agonist according to any one of the preceding items, wherein said peptide has an EC₅₀ towards the human GLP-2R that is at least 10 times lower than its EC₅₀ towards human GLP-1R.

45. The peptide dual agonist according to any one of the preceding items, wherein said peptide is selected from the group consisting of:

Compound no. 9
SEQ ID NO: 8
H-Aib-DG-K(γGlu-C16)-FISDYSTILDNLAARDFINWLIQTKITK-OH
Compound no. 10
SEQ ID NO: 9
H-Aib-DGTF-K(γGlu-C16)-SDYSTILDNLAARDFINWLIQTKITK-OH
Compound no. 11
SEQ ID NO: 10
H-Aib-DGTFISDY-K(γGlu-C16)-TILDNLAARDFINWLIQTKITK-OH
Compound no. 12
SEQ ID NO: 11
H-Aib-DGTFISDYS-K(γGlu-C16)-ILDNLAARDFINWLIQTKITK-OH
Compound no. 13
SEQ ID NO: 12
H-Aib-DGTFISDYSTIL-K(γGlu-C16)-NLAARDFINWLIQTKITK-OH
Compound no. 14
SEQ ID NO: 13
H-Aib-DGTFISDY-K(γGlu-C16)-TILDNLAARDFINWLIQTKGKKNDWKHNITQ-
NH₂
Compound no. 24
SEQ ID NO: 21
H-Aib-DG-K(γGlu-C10)-FISDYST-Aib-LDNLAARDFINWLIQTKITK-OH
Compound no. 25
SEQ ID NO: 21
H-Aib-DG-K(γGlu-C16 diacid)-FISDYST-Aib-LDNLAARDFINWLIQTKITK-OH
Compound no. 26
SEQ ID NO: 8
H-Aib-DG-K(γGlu-γGlu-C10)-FISDYSTILDNLAARDFINWLIQTKITK-OH
Compound no. 27
SEQ ID NO: 22
H-Aib-EG-K(γGlu-C16)-FISDYST-Aib-LDNLAARDFINWLIQTKITK-OH
Compound no. 28
SEQ ID NO: 21
H-Aib-DG-K(γGlu-γGlu-C16)-FISDYST-Aib-LDNLAARDFINWLIQTKITK-OH
Compound no. 29
SEQ ID NO: 23
H-Aib-DGTFISDY-K(γGlu-C16)-T-Aib-LDNLAARDFINWLIQTKITK-OH
Compound no. 30
SEQ ID NO: 25
H-Aib-DGTFISDY-K(γGlu-C10)-T-Aib-LDNLAARDFINWLIQTKITD-OH
Compound no. 31
SEQ ID NO: 26
H-Aib-DGTFISDY-K(γGlu-C10)-TILDKLAARDFIEWLIATKITK-OH
Compound no. 32
SEQ ID NO: 27
H-Aib-DGTFISDY-K(γGlu-C16)-TILDNLAARDFINWLIETKITK-OH
Compound no. 33
SEQ ID NO: 28
H-Aib-DGTFISDY-K(γGlu-C16)-T-Aib-LDALAARDFIEWLIQTKITK-OH
Compound no. 34
SEQ ID NO: 24
Ac-H-Aib-DGTFISDY-K(γGlu-C16)-T-Aib-LDNLAARDFINWLIQTKITK-OH
Compound no. 35
SEQ ID NO: 29
H-Aib-DGTFISDYS-K(γGlu-C16)-Aib-LDNLAARDFINWLIQTKITK-OH
Compound no. 36
SEQ ID NO: 30
Ac-H-Aib-DGTFISDYS-K(γGlu-C16)-Aib-LDNLAARDFINWLIQTKITK-OH
Compound no. 37
SEQ ID NO: 31
H-Aib-DGTFISDY-K(γGlu-C16)-TAib-LDNLAARDFINWLIQTKITKNDWKHNITQ-
OH
Compound no. 38
SEQ ID NO: 32
Ac-H-Aib-DGTFISDY-K(γGlu-C16)-T-Aib-
LDNLAARDFINWLIQTKITKNDWKHNITQ-OH
Compound no. 54
SEQ ID NO: 46
H-Aib-DGTFISDY-K(γglu-C10)-T-Aib-
LDNLAARDFINWLIQTKITGNDWKHNITQ-OH
Compound no. 1
SEQ ID NO: 1
(C16)HADGTFISDYSTILDNLAARDFINWLIQTKITD
Compound no. 2
SEQ ID NO: 2
(C16)HADGTFISDYSTILDNLAARDFINWLIQTKIT
Compound no. 45
SEQ ID NO: 38
(C16)HADGTFISDYSTILDNLAAKDFINWLIQTKITK
Compound no. 3
SEQ ID NO: 3
(C10)HADGTFISDYSTILDNLAAKDFINWLIQTKITKNDWKHNITQ
Compound no. 46
SEQ ID NO: 39
(C16)HADGTFISDYSTILDNLAARDFINWLLAQKITK
Compound no. 4
SEQ ID NO: 1
(C16)HADGTFISDYSTILDNLAARDFINWLIQTKITD
Compound no. 5
SEQ ID NO: 4
(C16)H-Aib-DGTFISDYST-Aib-LDNLAARDFINWLIQTKITK-OH
Compound no. 6
SEQ ID NO: 5
(C16)H-Aib-DGTFISDYSTILD-Aib-LAARDFINWLIQTKITK-OH
Compound no. 7
SEQ ID NO: 6
(C16)H-Aib-DGTFISDYSTILDNLAARDFIEWLIQTKITK-OH
Compound no. 8
SEQ ID NO: 7
(C16)H-Aib-DGTFISDYSTILDNLAARDFIEWLIQTKIT-OH
Compound no. 15
SEQ ID NO: 14
(C16)HADGTFISDYSTILELLAARDFINWLIQTKITKNDWKHNITQ
Compound no. 48
SEQ ID NO: 41
(C16)HADGTFISDYSTILDELAARDFINWLIQTKITKNDWKHNITQ
Compound no. 49
SEQ ID NO: 42
(C16)HADGTFISDYSTILDNLAARDFINWLIQTKITKAEWKHAITQ
Compound no. 50
SEQ ID NO: 43
(C16)HADGTFISDYSTILDNLAARDFIEWLIQTKITKNDWKHNITQ
Compound no. 51
SEQ ID NO: 44
(C16)HADGTFISDYSTILDNLAARDFINWLIETKITKNDWKHNITQ
Compound no. 52
SEQ ID NO: 45
(C16)HADGTFISDYSTILDNLAARDFINWLIATKITKNDWKHNITQ
Compound no. 53
SEQ ID NO: 33
(C16)HADGTFISDYSTAibLDNLAARDFINWLIQTKITKNDWKHNITQ
Compound no. 16
SEQ ID NO: 15
(C16)HADGTFISDYSTILDAibLAARDFINWLIQTKITKNDWKHNITQ
Compound no. 17
SEQ ID NO: 4
(C16-diacid)H-Aib-DGTFISDYST-Aib-LDNLAARDFINWLIQTKITK-OH
Compound no. 18
SEQ ID NO: 4
(C10-diacid)H-Aib-DGTFISDYST-Aib-LDNLAARDFINWLIQTKITK-OH
Compound no. 19
SEQ ID NO: 16
(C16-diacid)H-Aib-EGTFISDYST-Aib-LDNLAARDFINWLIQTKITK-OH
Compound no. 20
SEQ ID NO: 17
(C16-diacid)H-Aib-DGTFISDYST-Aib-LDALAARDFIEWLIQTKITK-OH
Compound no. 21
SEQ ID NO: 18
(C16-diacid) H-Aib-EGTFISDYST-Aib-LDKLAARDFINWLIQTKITK-OH
Compound no. 22
SEQ ID NO: 19
(C16-diacid)H-Aib-DGTFISDYST-Aib-LDKLAARDFIEWLIQTKITK-OH
Compound no. 23
SEQ ID NO: 20
(C16-diacid)H-Aib-DGTFISDYST-Aib-LDKLAARDFINWLIETKITK-OH
Compound no. 39
SEQ ID NO: 33
(C16-diacid)HADGTFISDYSTAibLDNLAARDFINWLIQTKITKNDWKHNITQ
Compound no. 40
SEQ ID NO: 34
(C16-diacid)H-Aib-DGTFISDYST-Aib-
LDNLAARDFINWLIQTKITKNDWKHNITQ-OH
Compound no. 41
SEQ ID NO: 35
(C16-diacid)H-Aib-EGTFISDYST-Aib-
LDALAARDFINWLIQTKITKNDWKHNITQ-OH
Compound no. 42
SEQ ID NO: 36
(C16-diacid)H-Aib-DGTFISDYST-Aib-
LDNLAAKDFINWLIQTKITKNDWKHNITQ-OH
Compound no. 43
SEQ ID NO: 37
(C16-diacid)H-Aib-EGTFISDYST-Aib-
LDNLAAKDFINWLIQTKITKNDWKHNITQ-OH,
and
Compound no. 44
SEQ ID NO: 4
(C10)H-Aib-DGTFISDYST-Aib-LDNLAARDFINWLIQTKITK-OH

46. The peptide dual agonist according to any one of the preceding items, wherein said peptide is selected from the group consisting of

SEQ ID NO: 4
(C16)H-Aib-DGTFISDYST-Aib-LDNLAARDFINWLIQTKITK-OH,
SEQ ID NO: 5
(C16)H-Aib-DGTFISDYSTILD-Aib-LAARDFINWLIQTKITK-OH,
Compound no. 9
SEQ ID NO: 8
H-Aib-DG-K(γGlu-C16)-FISDYSTILDNLAARDFINWLIQTKITK-OH,
Compound no. 10
SEQ ID NO: 10
H-Aib-DGTFISDY-K(γGlu-C16)-TILDNLAARDFINWLIQTKITK-OH,
SEQ ID NO: 11
H-Aib-DGTFISDYS-K(γGlu-C16)-ILDNLAARDFINWLIQTKITK-OH,
SEQ ID NO: 13
H-Aib-DGTFISDY-K(γGlu-C16)-TILDNLAARDFINWLIQTKGKKNDWKHNITQ-
NH2,
SEQ ID NO: 4
(C16-diacid)H-Aib-DGTFISDYST-Aib-LDNLAARDFINWLIQTKITK-OH,
SEQ ID NO: 16
(C16-diacid)H-Aib-EGTFISDYST-Aib-LDNLAARDFINWLIQTKITK-OH,
SEQ ID NO: 17
(C16-diacid)H-Aib-DGTFISDYST-Aib-LDALAARDFIEWLIQTKITK-OH,
SEQ ID NO: 19
(C16-diacid)H-Aib-DGTFISDYST-Aib-LDKLAARDFIEWLIQTKITK-OH,
SEQ ID NO: 21
H-Aib-DG-K(γGlu-C10)-FISDYST-Aib-LDNLAARDFINWLIQTKITK-OH,
SEQ ID NO: 21
H-Aib-DG-K(γGlu-C16 diacid)-FISDYST-Aib-LDNLAARDFINWLIQTKITK-OH,
SEQ ID NO: 23
H-Aib-DGTFISDY-K(γGlu-C16)-T-Aib-LDNLAARDFINWLIQTKITK-OH,
SEQ ID NO: 28
H-Aib-DGTFISDY-K(γGlu-C16)-T-Aib-LDALAARDFIEWLIQTKITK-OH,
SEQ ID NO: 31
H-Aib-DGTFISDY-K(γGlu-C16)-TAib-LDNLAARDFINWLIQTKITKNDWKHNITQ-
OH
SEQ ID NO: 34
(C16-diacid)H-Aib-DGTFISDYST-Aib-
LDNLAARDFINWLIQTKITKNDWKHNITQ-OH,
SEQ ID NO: 35
(C16-diacid)H-Aib-EGTFISDYST-Aib-
LDALAARDFINWLIQTKITKNDWKHNITQ-OH,
SEQ ID NO: 36
(C16-diacid)H-Aib-DGTFISDYST-Aib-
LDNLAAKDFINWLIQTKITKNDWKHNITQ-OH,
SEQ ID NO: 4
(C10)H-Aib-DGTFISDYST-Aib-LDNLAARDFINWLIQTKITK-OH,
and
SEQ ID NO: 26
H-Aib-DGTFISDY-K(γGlu-C10)-TILDKLAARDFIEWLIATKITK-OH.

47. A peptide dual agonist according to any of the preceding items for use as a medicament.

48. A peptide dual agonist according to any one of the preceding items for use in a method of inhibiting bone resorption and/or stimulating bone formation.

49. A peptide dual agonist according to any one of the preceding items for use in a method of treating a bone disorder.

50. The peptide dual agonist for use according to any one of the preceding items, wherein said bone disorder is selected from the group consisting of osteopenia, osteoporosis, severe osteoporosis, post-menopausal osteoporosis, iodiopathic osteoporosis, osteomalacia, rickets, osteitis fibrosa cystica (OFC) and Paget's disease of bone.

51. A peptide dual agonist according to any one of the preceding items for use in a method of treating osteoporosis in an individual suffering from Duchenne muscular dystrophy (DMD) or cerebral Palsy.

1.-51. (canceled)

52. A peptide dual agonist comprising or consisting of the sequence
(SEQ ID NO: 68)
HX₂X₃GX₅FX₇X₈X₉X₁₀X₁₁X₁₂X₁₃X₁₄X₁₅X₁₆LAAX₂₀DFIX₂₄WLIX₂₈TK
X₃₁X₃₂X₃₃-Z,
wherein
X₂ is Aib,
X₃ is D, K or E,
X₅ is T, K or S,
X₇ is K or I
X₈ is K or S
X₉ is D or K,
X₁₀ is Y or K,
X₁₁ is S, K or N,
X₁₂ is K or T
X₁₃ is A, Aib, K or I,
X₁₄ is L or K,
X₁₅ is K, D or E,
X₁₆ is E, L, Aib, K, A, N
X₂₀ is R or K,
X₂₄ is N or E,
X₂₈ is Q, E or A,
X₃₁ is I, G or omitted,
X₃₂ is T, K or omitted, and
X₃₃ is K, G, D or omitted,
wherein Z is a peptide comprising one or more amino acid residues of GIP (34-42) (NDWKHNITQ; SEQ ID NO: 58),
wherein said peptide is modified by attaching at least one fatty acid molecule at the N-terminus and/or at one or more Lysine or Ornithine residues at any one of positions 1 to 15 of SEQ ID NO: 68, and
wherein said peptide is an agonist of GIPR (glucose-dependent insulinotropic polypeptide receptor) and is an agonist of GLP-2R (glucagon-like peptide-2 receptor).

53. The peptide dual agonist according to claim 52, wherein said peptide comprises or consists of the sequence
(SEQ ID NO: 69)
HX₂X₃GX₅FIX₈X₉X₁₀X₁₁X₁₂X₁₃LX₁₅X₁₆LAAX₂₀DFIX₂₄WLIX₂₈TKX₃₁
X₃₂X₃₃-Z ,
wherein
X₂ is Aib,
X₃ is D or E,
X₅ is T, K or S,
X₈ is K or S
X₉ is D or K,
X₁₀ is Y or K,
X₁₁ is S or K,
X₁₂ is K or T
X₁₃ is Aib or I,
X₁₅ is D or E,
X₁₆ is E, L, Aib, K, A, N
X₂₀ is R or K,
X₂₄ is N or E,
X₂₈ is Q, E or A,
X₃₁ is I, G or omitted,
X₃₂ is T or omitted, and
X₃₃ is K, G, D or omitted.

54. The peptide dual agonist according claim 52, wherein said peptide is modified by attaching at least one fatty acid molecule at a Lysine residue at any one of positions 2 to 15 of SEQ ID NO: 68.

55. The peptide dual agonist according to claim 52, wherein X₇ is I, and wherein said peptide is modified by attaching one fatty acid molecule at position 1 of SEQ ID NO: 68.

56. The peptide dual agonist according to claim 52, wherein X₂ is Aib and X₁₃ is Aib, X₂ is Aib and X₁₁ is K, X₂ is Aib, X₅ is K and X₁₃ is Aib, X₂ is Aib, X₁₁ is K and X₁₃ is Aib, or X₂ is Aib, X₁₁ is K and X₂₄ is E.

57. The peptide dual agonist according to claim 52, wherein Z is a peptide selected from:
ND, NDW, KDW, NDW, NDK, NDWK (SEQ ID NO: 59), NDWKH (SEQ ID NO: 60), NDWKHN (SEQ ID NO: 61), NDWKHNI (SEQ ID NO: 62), NDWKHNIT (SEQ ID NO: 63), NDWKHNITQ (SEQ ID NO: 64), and AEWKHAITQ (SEQ ID NO: 65) or a variant comprising one amino acid substitution.

58. The peptide dual agonist according to claim 52, wherein said peptide is
Compound no. 9
SEQ ID NO: 8
H-Aib-DGKFISDYSTILDNLAARDFINWLIQTKITK,
Compound no. 10
SEQ ID NO: 9
H-Aib-DGTFKSDYSTILDNLAARDFINWLIQTKITK,
Compound no. 11
SEQ ID NO: 10
H-Aib-DGTFISDYKTILDNLAARDFINWLIQTKITK,
Compound no. 12
SEQ ID NO: 11
H-Aib-DGTFISDYSKILDNLAARDFINWLIQTKITK,
Compound no. 13
SEQ ID NO: 12
H-Aib-DGTFISDYSTILKNLAARDFINWLIQTKITK,
Compound no. 14
SEQ ID NO: 13
H-Aib-DGTFISDYKTILDNLAARDFINWLIQTKGKKNDWKHNITQ,
Compound no. 24
SEQ ID NO: 21
H-Aib-DGKFISDYST-Aib-LDNLAARDFINWLIQTKITK,
Compound no. 25
SEQ ID NO: 21
H-Aib-DGKFISDYST-Aib-LDNLAARDFINWLIQTKITK,
Compound no. 26
SEQ ID NO: 8
H-Aib-DGKFISDYSTILDNLAARDFINWLIQTKITK,
Compound no. 27
SEQ ID NO: 22
H-Aib-EGKFISDYST-Aib-LDNLAARDFINWLIQTKITK,
Compound no. 28
SEQ ID NO: 21
H-Aib-DGKFISDYST-Aib-LDNLAARDFINWLIQTKITK,
Compound no. 29
SEQ ID NO: 23
H-Aib-DGTFISDYKT-Aib-LDNLAARDFINWLIQTKITK,
Compound no. 30
SEQ ID NO: 25
H-Aib-DGTFISDYKT-Aib-LDNLAARDFINWLIQTKITD,
Compound no. 31
SEQ ID NO: 26
H-Aib-DGTFISDYKTILDKLAARDFIEWLIATKITK,
Compound no. 32
SEQ ID NO: 27
H-Aib-DGTFISDYKTILDNLAARDFINWLIETKITK,
Compound no. 33
SEQ ID NO: 28
H-Aib-DGTFISDYKT-Aib-LDALAARDFIEWLIQTKITK,
Compound no. 34
SEQ ID NO: 24
Ac-H-Aib-DGTFISDYKT-Aib-LDNLAARDFINWLIQTKITK,
Compound no. 35
SEQ ID NO: 29
H-Aib-DGTFISDYSKAib-LDNLAARDFINWLIQTKITK,
Compound no. 36
SEQ ID NO: 30
Ac-H-Aib-DGTFISDYSKAib-LDNLAARDFINWLIQTKITK,
Compound no. 37
SEQ ID NO: 31
H-Aib-DGTFISDYKTAib-LDNLAARDFINWLIQTKITKNDWKHNIT
Q,
Compound no. 38
SEQ ID NO: 32
Ac-H-Aib-DGTFISDYKT-Aib-LDNLAARDFINWLIQTKITKNDWKH
NITQ,
Compound no. 54
SEQ ID NO: 46
H-Aib-DGTFISDYKT-Aib-LDNLAARDFINWLIQTKITGNDWKHNI
TQ,
Compound no. 5
SEQ ID NO: 4
H-Aib-DGTFISDYST-Aib-LDNLAARDFINWLIQTKITK,
Compound no. 6
SEQ ID NO: 5
H-Aib-DGTFISDYSTILD-Aib-LAARDFINWLIQTKITK,
Compound no. 7
SEQ ID NO: 6
H-Aib-DGTFISDYSTILDNLAARDFIEWLIQTKITK,
Compound no. 8
SEQ ID NO: 7
H-Aib-DGTFISDYSTILDNLAARDFIEWLIQTKIT,
Compound no. 17
SEQ ID NO: 4
H-Aib-DGTFISDYST-Aib-LDNLAARDFINWLIQTKITK,
Compound no. 19
SEQ ID NO: 16
H-Aib-EGTFISDYST-Aib-LDNLAARDFINWLIQTKITK,
Compound no. 20
SEQ ID NO: 17
H-Aib-DGTFISDYST-Aib-LDALAARDFIEWLIQTKITK,
Compound no. 21
SEQ ID NO: 18
H-Aib-EGTFISDYST-Aib-LDKLAARDFINWLIQTKITK,
Compound no. 22
SEQ ID NO: 19
H-Aib-DGTFISDYST-Aib-LDKLAARDFIEWLIQTKITK,
Compound no. 23
SEQ ID NO: 20
H-Aib-DGTFISDYST-Aib-LDKLAARDFINWLIETKITK,
Compound no. 39
SEQ ID NO: 33
HADGTFISDYSTAibLDNLAARDFINWLIQTKITKNDWKHNITQ,
Compound no. 40
SEQ ID NO: 34
H-Aib-DGTFISDYST-Aib-LDNLAARDFINWLIQTKITKNDWKHNI
TQ,
Compound no. 41
SEQ ID NO: 35
H-Aib-EGTFISDYST-Aib-LDALAARDFINWLIQTKITKNDWKHNI
TQ,
Compound no. 42
SEQ ID NO: 36
H-Aib-DGTFISDYST-Aib-LDNLAAKDFINWLIQTKITKNDWKHNI
TQ,
Compound no. 43
SEQ ID NO: 37
H-Aib-EGTFISDYST-Aib-LDNLAAKDFINWLIQTKITKNDWKHNI
TQ,
and
Compound no. 44
SEQ ID NO: 4
H-Aib-DGTFISDYST-Aib-LDNLAARDFINWLIQTKITK.
or a functional variant thereof, wherein said functional variant comprises 1 or 2 individual amino acid substitutions.

59. The peptide dual agonist according to claim 58,
wherein said functional variant comprises 1 to 3 individual amino acid substitutions with the proviso that the amino acid residue at position 2 of said functional variant is Aib,
the amino acid residue at position 6 of said functional variant is Phenylalanine (F),
the amino acid residue at position 22 of said functional variant is Phenylalanine (F),
the amino acid residue at position 25 of said functional variant is Tryptophan (W), and
the amino acid residue at position 26 of said functional variant is Leucine (L).

60. The peptide dual agonist according to claim 52, wherein said peptide is C-terminally amidated (—NH₂) or wherein the C-terminus is a carboxylic acid.

61. The peptide dual agonist according to claim 52, wherein X₁₃ is Aib, and wherein said peptide is modified by attaching at least one fatty acid molecule at any one of positions 5 or 11 of SEQ ID NO: 68.

62. The peptide dual agonist according to claim 52, wherein said fatty acid molecule is a straight-chain fatty acid or wherein said fatty acid molecule is a branched fatty acid.

63. The peptide dual agonist according to claim 52, wherein said fatty acid molecule is a monoacyl fatty acid molecule, or wherein said fatty acid molecule is a diacyl fatty acid molecule.

64. The peptide dual agonist according to claim 52, wherein said fatty acid molecule comprises an acyl group selected from CH₃(CH₂)₈CO— (capriyl, C10), CH₃(CH₂)₁₀CO— (lauryl, C12), CH₃(CH₂)₁₂CO— (myristoyl, C14), CH₃(CH₂)₁₄CO— (palmitoyl, C16), CH₃(CH₂)₁₆CO— (stearyl, C18) and CH₃(CH₂)₁₈CO— (arachidyl, C20), or wherein said fatty acid molecule comprises two acyl groups individually selected from HOOC—CH₃(CH₂)₈CO— (decanoyl, C10), HOOC—CH₃(CH₂)₁₀CO— (dodecanoyl, C12), HOOC—CH₃(CH₂)₁₂CO— (1-tetradecanoyl, C14), HOOC—CH₃(CH₂)₁₄CO— (hexadecanoyl, C16), HOOC—CH₃(CH₂)₁₅CO— (15-carboxy-pentadecanoyl, C17), HOOC—CH₃(CH₂)₁₆CO— (octadecanoyl, C18), HOOC—CH₃(CH₂)₁₇CO— (17-carboxy-heptadecanoyl, C19), HOOC—CH₃(CH₂)₁₈CO— (eicosanoyl, C20), and HOOC—CH₃(CH₂)₁₉CO— (19-carboxy-nonadecanoyl, C21).

65. The peptide dual agonist according to claim 52, wherein said fatty acid molecule is attached to an amino acid residue via a spacer.

66. The peptide dual agonist according to claim 65, wherein said spacer comprises or consists of yGlu or yGlu-yGlu.

67. The peptide dual agonist according to claim 52, wherein said peptide is:
Compound no. 9
SEQ ID NO: 8
H-Aib-DG-K(γGlu-C16)-FISDYSTILDNLAARDFINWLIQTKITK-OH
Compound no. 10
SEQ ID NO: 9
H-Aib-DGTF-K(γGlu-C16)-SDYSTILDNLAARDFINWLIQTKITK-OH
Compound no. 11
SEQ ID NO: 10
H-Aib-DGTFISDY-K(γGlu-C16)-TILDNLAARDFINWLIQTKITK-OH
Compound no. 12
SEQ ID NO: 11
H-Aib-DGTFISDYS-K(γGlu-C16)-ILDNLAARDFINWLIQTKITK-OH
Compound no. 13
SEQ ID NO: 12
H-Aib-DGTFISDYSTIL-K(γGlu-C16)-NLAARDFINWLIQTKITK-OH
Compound no. 14
SEQ ID NO: 13
H-Aib-DGTFISDY-K(γGlu-C16)-TILDNLAARDFINWLIQTKGKKNDWKHNITQ-
NH₂
Compound no. 24
SEQ ID NO: 21
H-Aib-DG-K(γGlu-C10)-FISDYST-Aib-LDNLAARDFINWLIQTKITK-OH
Compound no. 25
SEQ ID NO: 21
H-Aib-DG-K(γGlu-C16 diacid)-FISDYST-Aib-LDNLAARDFINWLIQTKITK-OH
Compound no. 26
SEQ ID NO: 8
H-Aib-DG-K(γGlu-γGlu-C10)-FISDYSTILDNLAARDFINWLIQTKITK-OH
Compound no. 27
SEQ ID NO: 22
H-Aib-EG-K(γGlu-C16)-FISDYST-Aib-LDNLAARDFINWLIQTKITK-OH
Compound no. 28
SEQ ID NO: 21
H-Aib-DG-K(γGlu-γGlu-C16)-FISDYST-Aib-LDNLAARDFINWLIQTKITK-OH
Compound no. 29
SEQ ID NO: 23
H-Aib-DGTFISDY-K(γGlu-C16)-T-Aib-LDNLAARDFINWLIQTKITK-OH
Compound no. 30
SEQ ID NO: 25
H-Aib-DGTFISDY-K(γGlu-C10)-T-Aib-LDNLAARDFINWLIQTKITD-OH
Compound no. 31
SEQ ID NO: 26
H-Aib-DGTFISDY-K(γGlu-C10)-TILDKLAARDFIEWLIATKITK-OH
Compound no. 32
SEQ ID NO: 27
H-Aib-DGTFISDY-K(γGlu-C16)-TILDNLAARDFINWLIETKITK-OH
Compound no. 33
SEQ ID NO: 28
H-Aib-DGTFISDY-K(γGlu-C16)-T-Aib-LDALAARDFIEWLIQTKITK-OH
Compound no. 34
SEQ ID NO: 24
Ac-H-Aib-DGTFISDY-K(γGlu-C16)-T-Aib-LDNLAARDFINWLIQTKITK-OH
Compound no. 35
SEQ ID NO: 29
H-Aib-DGTFISDYS-K(γGlu-C16)-Aib-LDNLAARDFINWLIQTKITK-OH
Compound no. 36
SEQ ID NO: 30
Ac-H-Aib-DGTFISDYS-K(γGlu-C16)-Aib-LDNLAARDFINWLIQTKITK-OH
Compound no. 37
SEQ ID NO: 31
H-Aib-DGTFISDY-K(γGlu-C16)-TAib-LDNLAARDFINWLIQTKITKNDWKHNITQ-
OH
Compound no. 38
SEQ ID NO: 32
Ac-H-Aib-DGTFISDY-K(yGlu-C16)-T-Aib-
LDNLAARDFINWLIQTKITKNDWKHNITQ-OH
Compound no. 54
SEQ ID NO: 46
H-Aib-DGTFISDY-K(yglu-C10)-T-Aib-LDNLAARDFINWLIQTKITGNDWKHNITQ-
OH
Compound no. 5
SEQ ID NO: 4
(C16)H-Aib-DGTFISDYST-Aib-LDNLAARDFINWLIQTKITK-OH
Compound no. 6
SEQ ID NO: 5
(C16)H-Aib-DGTFISDYSTILD-Aib-LAARDFINWLIQTKITK-OH
Compound no. 7
SEQ ID NO: 6
(C16)H-Aib-DGTFISDYSTILDNLAARDFIEWLIQTKITK-OH
Compound no. 8
SEQ ID NO: 7
(C16)H-Aib-DGTFISDYSTILDNLAARDFIEWLIQTKIT-OH
Compound no. 17
SEQ ID NO: 4
(C16-diacid)H-Aib-DGTFISDYST-Aib-LDNLAARDFINWLIQTKITK-OH
Compound no. 18
SEQ ID NO: 4
(C10-diacid)H-Aib-DGTFISDYST-Aib-LDNLAARDFINWLIQTKITK-OH
Compound no. 19
SEQ ID NO: 16
(C16-diacid)H-Aib-EGTFISDYST-Aib-LDNLAARDFINWLIQTKITK-OH
Compound no. 20
SEQ ID NO: 17
(C16-diacid)H-Aib-DGTFISDYST-Aib-LDALAARDFIEWLIQTKITK-OH
Compound no. 21
SEQ ID NO: 18
(C16-diacid)H-Aib-EGTFISDYST-Aib-LDKLAARDFINWLIQTKITK-OH
Compound no. 22
SEQ ID NO: 19
(C16-diacid)H-Aib-DGTFISDYST-Aib-LDKLAARDFIEWLIQTKITK-OH
Compound no. 23
SEQ ID NO: 20
Compound no. 40
SEQ ID NO: 34
(C16-diacid)H-Aib-DGTFISDYST-Aib-LDNLAARDFINWLIQTKITKNDWKHNITQ-
OH
Compound no. 41
SEQ ID NO: 35
(C16-diacid)H-Aib-EGTFISDYST-Aib-LDALAARDFINWLIQTKITKNDWKHNITQ-
OH
Compound no. 42
SEQ ID NO: 36
(C16-diacid)H-Aib-DGTFISDYST-Aib-LDNLAAKDFINWLIQTKITKNDWKHNITQ-
OH
Compound no. 43
SEQ ID NO: 37
(C16-diacid)H-Aib-EGTFISDYST-Aib-LDNLAAKDFINWLIQTKITKNDWKHNITQ-
OH,
or
Compound no. 44
SEQ ID NO: 4
(C10)H-Aib-DGTFISDYST-Aib-LDNLAARDFINWLIQTKITK-OH,
or a functional variant thereof, wherein said functional variant comprises 1 or 2 individual amino acid substitutions.

68. A method for treatment of a bone disorder comprising administration of a therapeutically effective amount of a peptide dual agonist according claim 1 to an individual in need thereof.

69. The method according to claim 68, wherein said bone disorder is osteopenia, osteoporosis, severe osteoporosis, post-menopausal osteoporosis, idiopathic osteoporosis, osteomalacia, rickets, osteitis fibrosa cystica (OFC) or Paget's disease of bone.

70. A method according to claim 69, wherein said bone disorder is osteoporosis in an individual suffering from Duchenne muscular dystrophy (DMD) or cerebral Palsy.
